(19) United States
(12) Patent Application Publication
(10) Pub. No.: US 2009/0298707 A1
(43) Pub. Date: Dec. 3, 2009

(54) SPARSE MATRIX SYSTEM AND METHOD FOR IDENTIFICATION OF SPECIFIC LIGANDS OR TARGETS

(75) Inventors: Daniel K. Yarbrough, Los Angeles, CA (US); Randal H. Eckert, Redondo Beach, CA (US); Ben Wu, San Marino, CA (US); Elizabeth M. Hagerman, Beverly Hills, CA (US); Wenyuan Shi, Los Angeles, CA (US); Maxwell H. Anderson, Sequim, WA (US); Fengxia Qi, Edmond, OK (US); Jian He, Los Angeles, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: The Regents of the University of California, Oakland, CA (US); C3 Jian, Inc., Ingelwood, CA (US)

(21) Appl. No.: 12/406,856

(22) Filed: Mar. 18, 2009

Related U.S. Application Data
(60) Provisional application No. 61/037,603, filed on Mar. 18, 2008.

(51) Int. Cl. C40B 30/04; C40B 40/12; C40B 40/10; C40B 40/14; C40B 40/02; C40B 50/06; C07K 7/00

(52) U.S. Cl. 506/9; 506/19; 506/18; 506/20; 506/14; 506/26; 530/328

(57) ABSTRACT

In certain embodiments, this invention pertains to the creation of “Sparse Matrix” libraries of compounds. The sparse matrix libraries of this invention typically span an n-dimensional parameter (property) space at extremely sparse intervals and thereby provide a relatively small library of compounds (e.g., a library of about 200 or fewer compounds) that can be used to quickly and efficiently screen the compound parameter space for one or more desired physical, chemical, or biological properties (e.g., binding specificity, binding avidity, γtoxicity, solubility, mobility, cell permeability, serum half-life, biocompatibility, etc.).

### CROSS-REFERENCE TO RELATED APPLICATIONS

This application claims priority to and benefit of U.S. Ser. No. 61/037,603, filed Mar. 18, 2008, which is incorporated herein by reference in its entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention relates to the field of peptide engineering. In particular, a novel platform for identifying peptides having particular properties is disclosed.

### BACKGROUND OF THE INVENTION

Bioactive peptides currently enjoy considerable interest as reagents in research and biotechnology (Tomizaki et al. (2005) Chembiochem 6(5): 782-799; Tozzi and Giraudi (2006) Curr Pharm Des 12(2): 191-203; Reddy and Kodadek (2005) Proc. Natl. Acad. Sci., USA, 102 (36): 12672-12677), for vaccine development (Klemm and Schembri (2000) Microbiology 146 Pt 12: 3025-3032; Yang et al. (2006) Vaccine 24(8): 1117-1123), and as drug candidates for the treatment of conditions as diverse as HIV infection (Kazmierskiet al. (2006) Chem Biol Drug Des 67(1): 13-26), cancer (Hu et al. (2006) Biochem. Biophys. Res. Commun., 341(4): 964-972; Shadidi and Sioud (2003) Drug Resist Updat 6(6): 363-371), and bacterial infections (Reddy et al. (2004) Int J Antimicrob Agents 24(6): 536-547; Riley and Wertz (2002) Annu Rev Microbiol 56: 117-137). Their many potential uses, combined with their low relative cost and ease of synthesis has created enormous demand for novel peptides. The wide variety of targets requires that multiple methods be available to generate, screen, and select from the various types of peptide libraries. As each application brings with it unique challenges, the past two decades have seen the development of several techniques for producing, displaying, and screening peptides for almost every purpose (Falciani et al. (2005) Chem Biol 12(4): 417-426; Georgiou et al. (1997) Nat Biotechnol 15(1): 29-34; Clackson and Wells (1994) Trends Biotechnol 12(5): 173-184; Meloen et al. (2004) Mol Divers 8(2): 57-59; Buts et al. (2003) Mol Microbiol 49(3): 705-715).

The primary goal of most peptide library screening is to identify peptides that mediate specific, high-affinity interactions with a chosen target receptor. Isolated receptor proteins present a relatively limited number of surface epitopes, thus it is unlikely that any given sequence will interact with the target in the desired manner. For this reason, obtaining receptor-binding or protein-binding peptides typically required the screening of either very large random libraries or smaller biased libraries based on highly specific structural information in order to reliably obtain peptides of interest.

### SUMMARY OF THE INVENTION

Receptors, and other cell-surface “markers” occur within the context of the cell surface, a complex collection of lipids, proteins, and polysaccharides. This surface can alter the mechanism by which a peptide might interact with the marker and/or provide a wide variety of other unique binding sites for peptide interaction. Accordingly, it is difficult and sometimes impossible to a priori predict binding dynamics.

It was a discovery that small, rationally designed libraries of linear polymers (e.g., peptides) that are of manageable size but that still cover broad areas of the parameter space can readily be used to successfully identify polymers that specifically interact with the biological surfaces and/or targets. Typically the backbone of the “linear” polymer is constrained by the nature of the polymer in these compounds and the chemical diversity is generated by the sidechains/subunits selection. This makes it possible to design such libraries that vary in chosen parameters in a rational, stepwise fashion and surprisingly, such small libraries are highly effective tools for identifying binding moieties against a wide variety of targets.

Accordingly, in certain embodiments multidimensional sparse matrix library of compounds is provided. The library typically comprises a dimensionality D where D is the number of different compound properties systematically varied in the library and D ranges from about 2 to about 100, preferably from about 2 to about 50, more preferably from about 20 to about 25, and most preferably from about 2 to about 20, 15, 10, 8, or 6; where compounds comprising the library are characterized by different properties P₁, P₂, through P_{D} and each compound comprising the library can be designated as C_{P1, P2, P3, . . . PD}, such that compounds designated with the same subscripts P₁, P₂, through P_{D} have essentially the same values for the properties identified with those subscripts and vary with respect to the remaining properties, and where the library comprises no more than about 500, preferably no more than about 400 or 300, more preferably no more than about 200 or 100, and most preferably no more than about 80, 50, 40, or 30 members that differ with respect to each property. In certain embodiments the library is a 2-dimensional library (D=2) and where the library comprises: an m×n matrix of a plurality of compounds, where m and n are integers, 2≦m≦100, and 2≦n≦100; where compounds comprising the library are characterized by a first property and a second property and can be designated as C_{i,j}, where 1≦i≦m, and 1≦j≦n, and compounds having the same value i and different values j have essentially the same value of the first property and vary with respect to the second property, while compounds having the same value j and different values i have essentially the same value of the second property and vary with respect to the first property. In certain embodiments the compounds are polymeric biomolecules, but are not nucleic acids. In certain embodiments the compounds are selected from the group consisting of polysaccharides, lipid polymers, and peptides. In certain embodiments the compounds are linear polymers (e.g., peptides and/or peptoids). In certain embodiments the compounds are natural peptides (i.e., peptides composed of naturally occurring amino acids). In certain embodiments the first property and second property are different properties and are independently selected from the group consisting of hydrophobicity, charge, length, periodicity, molecular weight, stokes radius, van der Waals radius, conformational flexibility, aromaticity, aliphatic index, helix-forming potential in aqueous solution, helix-forming potential in hydrophobic environments, dipole moment, sheet forming potential, polarizability, or any ratio of these parameters. In certain embodiments the first property is hydrophobicity, and the second property is charge. In certain embodiments the peptide and/or peptoids in the library comprises about 4 to about 200, about 150, about 100, about 50, about 40, about 30, about 20, about 15, about 10, about 8, or about 6 amino acids. In certain embodiments the peptide and/or peptoids in the library comprises about 4 to about 20 amino acids. In certain embodiments the library (e.g., a 2-dimensional, 3-dimensional, 4 dimensional, 5-dimensional, 6-dimensional, 7 dimensional, 8-dimensional, 9-dimensional, 10-dimensional or greater library) comprises ≦about 10,000, ≦about 5,000, ≦about 4,000, ≦about 3,000, ≦about 2,000, ≦about 1,000, ≦about 900, ≦about 800, ≦about 700, ≦about 600, ≦about 500, ≦about 400, ≦about 300, ≦about 200, ≦about 100, ≦about 90, ≦about 80, ≦about 70, ≦about 60, ≦about 50, ≦about 40, ≦about 30, ≦about 20, or ≦about 10 different compounds. In certain embodiments the library is a 2-dimensional library comprising ≦about 10,000, ≦about 5,000, ≦about 4,000, ≦about 3,000, ≦about 2,000, ≦about 1,000, ≦about 900, ≦about 800, ≦about 700, ≦about 600, ≦about 500, ≦about 400, ≦about 300, ≦about 200, ≦about 100, ≦about 90, ≦about 80, ≦about 70, ≦about 60, ≦about 50, ≦about 40, ≦about 30, ≦about 20, or ≦about 10 different compounds. In certain embodiments 2≦m≦100, 2≦m≦90, 2≦m≦80, 2≦m≦70, 2≦m≦60, 2≦m≦50, 2≦m≦40, 2≦m≦30, 2≦m≦20, or 2≦m≦10. In certain embodiments m is 10, 9, 8, 7, 6, 5, 4, 3, or 2. In certain embodiments 2≦n≦100, 2≦n≦90, 2≦n≦80, 2≦n≦70, 2≦n≦60, 2≦n≦50, 2≦n≦40, 2≦n≦30, 2≦n≦20, or 2≦n≦10. In certain embodiments, n is 10, 9, 8, 7, 6, 5, 4, 3, or 2. In various embodiments members of the library are labeled with a detectable label (e.g., a radiometric label, a spin label, a fluorescent label, a calorimetric label, an enzymatic label, a quantum dot, a nanoparticle, a magnetic label, etc.). In certain embodiments members of the library are attached to a solid substrate (e.g., bead(s), a contiguous solid substrate, etc.). In certain embodiments members of the library are spatially addressed so the identity of a member of the library can be determined by its location on the substrate. In certain embodiments members of the library are disposed on different substrates. In certain embodiments different members of the library are disposed in different containers or receptacles. In certain embodiments different members of the library are disposed in different wells of one or more microtiter plates. In certain embodiments different members of the library are disposed in containers containing cultures of cells, bacteria, protozoa, fungi, algae, or virus, where different containers contain different members of the library.

In various embodiments a peptide matrix is provided comprising: an m×n matrix of a plurality of peptides, where m and n are integers, 2≦m≦100, and 2≦n≦100; where one the peptide in the matrix is designated as P_{i,j}≦1≦i≦m, and 1≦j≦n; where each the peptide P_{i,j} is characterized by having a first parameter and a second parameter; where a column of the peptides along the longitudinal direction in the matrix differ with each other in the value of the first parameter; and where a row of the peptides along the latitudinal direction differ with each other the value of the second parameter. In certain embodiments the peptide(s) comprise about 4 to about 200, about 150, about 100, about 50, about 40, about 30, about 20, about 15, about 10, about 8, or about 6 amino acids. In certain embodiments the peptides comprise about 4 to about 20 amino acids. In certain embodiments the matrix comprises about 4, 6, 8, 10, 12, 15, 20, 25, 30, 35, 40, or 50 to less than about 10,000, less than about 5,000, less than about 3, less than about 000, less than about 2, less than about 000, less than about 1, less than about 000, less than about 900, less than about 800, less than about 700, less than about 600, less than about 500, less than about 400, less than about 300, less than about 200, less than about 100, less than about 90, less than about 80, less than about 70, less than about 60, less than about f0, less than about 40, less than about 30, less than about 20, less than about, or less than about 10 different peptides. In certain embodiments the m×n matrix comprises ≦about 10,000, ≦about 5,000, ≦about 4,000, ≦about 3,000, ≦about 2,000, ≦about 1,000, ≦about 900, ≦about 800, ≦about 700, ≦about 600, ≦about 500, ≦about 400, ≦about 300, ≦about 200, ≦about 100, ≦about 90, ≦about 80, ≦about 70, ≦about 60, ≦about 50, ≦about 40, ≦about 30, ≦about 20, or ≦about 10 peptides. In certain embodiments 2≦m≦100, 2≦m≦90, 2≦m≦80, 2≦m≦70, 2≦m≦60, 2≦m≦50, 2≦m≦40, 2≦m≦30, 2≦m≦20, or 2≦m≦10. In certain embodiments m is 10, 9, 8, 7, 6, 5, 4, 3, or 2. In certain embodiments 2≦n≦100, 2≦n≦90, 2≦n≦80, 2≦n≦70, 2≦n≦60, 2≦n≦50, 2≦n≦40, 2≦n≦30, 2≦n≦20, or 2≦n≦10. In certain embodiments n is 10, 9, 8, 7, 6, 5, 4, 3, or 2. In certain embodiments the first parameter is hydrophobicity and the second parameter is charge. In certain embodiments the peptide p_{1,1} possess a high (or low) end value of hydrophobicity and a high (or low) end value of charge, p_{m,1} possess the low (or high) end value of hydrophobicity and the high (or low) end value of charge, p_{1,n} possesses the high (or low) end value of hydrophobicity and the low (or high) end value of charge, and p_{m,n} possess the low (or high) end value of hydrophobicity and the low (of high) end value of charge. In certain embodiments the first parameter is charge and the second parameter is hydrophobicity. In certain embodiments the peptide p_{1,1} possess a high (or low) end value of charge and a high (or low) end value of hydrophobicity, p_{m,1} possess the low (or high) end value of charge and the high (or low) end value of hydrophobicity, p_{1,n} possesses the high (or low) end value of charge and the low (or high) end value of hydrophobicity, and p_{m,n} possess the low (or high) end value of charge and the low (of high) end value of hydrophobicity. In certain embodiments the values of the first parameter in the peptides along the longitudinal direction changes in a stepwise fashion. In certain embodiments values of the second parameter in the peptides along the latitudinal direction changes in a stepwise fashion.

In certain embodiments methods are provided of preparing an m×n peptide matrix, where the matrix comprises m×n peptides, where m and n are integers, 2≦m≦100, and 2≦n≦100; where one the peptide in the matrix is designated as P_{i,j}, 1≦i≦m, and 1≦j≦n; where each the peptide P_{i,j} is characterized by having a first parameter and a second parameter; where a column of the peptides along the longitudinal direction in the matrix differ with each other in the value of the first parameter; and where a row of the peptides along the latitudinal direction differ with each other the value of the second parameter; the method comprising: i) designating a length of the peptides; ii) designating a desired scope of the first parameter and the second parameter to be sampled; iii) providing a first step size for the first parameter and a second step size for the second parameter or providing a size of the matrix; iv) providing at least a first peptide possessing a first value for the first parameter and a second value for the second parameter; v) changing, substituting, or alternating the amino acids from the first peptide such that the peptides along the longitudinal direction in the matrix differ with each other in the value of the first parameter and the peptides along the latitudinal direction differ with each other the value of the second parameter; and synthesizing the peptides comprising said library and/or placing the peptides comprising said library in one or more receptacles, and/or contacting members of said library with one or more target microorganisms, cells, or tissues. In certain embodiments the method further comprises providing a second peptide, a third peptide, and a fourth peptide: where the first peptide is p_{1,1} possessing the first value which is a high (or low) end value of the first parameter and the second value which is a high (or low) end value of a second parameter, where the second peptide is p_{m,1} possessing the low (or high) end value of the first parameter and the high (or low) end value of the second parameter, where the third peptide is p_{1,n} possessing the high (or low) end value of the first parameter and the low (or high) end value of the second parameter, and where the fourth peptide is p_{m,n} possessing the low (or high) end value of the first parameter and the low (of high) end value of the second parameter. In certain embodiments the first parameter is hydrophobicity (or charge) and the second parameter is charge (or hydrophobicity). In certain embodiments the peptide(s) of the library comprise about 4 to about 200, about 150, about 100, about 50, about 40, about 30, about 20, about 15, about 10, about 8, or about 6 amino acids. In certain embodiments the peptides comprise about 4 to about 20 amino acids. In certain embodiments the matrix comprises about 4, 6, 8, 10, 12, 15, 20, 25, 30, 35, 40, or 50 to less than about 10,000, less than about 5,000, less than about 3, less than about 000, less than about 2, less than about 000, less than about 1, less than about 000, less than about 900, less than about 800, less than about 700, less than about 600, less than about 500, less than about 400, less than about 300, less than about 200, less than about 100, less than about 90, less than about 80, less than about 70, less than about 60, less than about f0, less than about 40, less than about 30, less than about 20, less than about, or less than about 10 different peptides. In certain embodiments the m×n matrix comprises ≦about 10,000, ≦about 5,000, ≦about 4,000, ≦about 3,000, ≦about 2,000, ≦about 1,000, ≦about 900, ≦about 800, ≦about 700, ≦about 600, ≦about 500, ≦about 400, ≦about 300, ≦about 200, ≦about 100, ≦about 90, ≦about 80, ≦about 70, ≦about 60, ≦about 50, ≦about 40, ≦about 30, ≦about 20, or ≦about 10 peptides. In certain embodiments 2≦m≦100, 2≦m≦90, 2≦m≦80, 2≦m≦70, 2≦m≦60, 2≦m≦50, 2≦m≦40, 2≦m≦30, 2≦m≦20, or 2≦m≦10. In certain embodiments m is 10, 9, 8, 7, 6, 5, 4, 3, or 2. In certain embodiments 2≦n≦100, 2≦n≦90, 2≦n≦80, 2≦n≦70, 2≦n≦60, 2≦n≦50, 2≦n≦40, 2≦n≦30, 2≦n≦20, or 2≦n≦10. In certain embodiments n is 10, 9, 8, 7, 6, 5, 4, 3, or 2.

In certain embodiments methods are provided of identifying a compound that specifically interacts with a target. The methods typically involve i) providing a sparse matrix library as described above and below herein; ii) contacting the compounds comprising the matrix to a target; and iii) identifying and/or selecting the compounds that specifically and/or preferentially bind to the target. In certain embodiments the method further involves generating a second peptide matrix using peptides selected or identified as specifically or preferentially binding the target. In certain embodiments the target is selected from the group consisting of a biological surface, a microorganism, a prokaryotic cell, a eukaryotic cell, a receptor, and a compound. In certain embodiments the target is selected from the group consisting of a yeast, a protozoan, a bacterium, an archaea, a virus, a fungus, and an alga.

In certain embodiments methods are provided of identifying a pattern of interaction between a target and a sparse matrix library. The methods typically involve i) providing a sparse matrix as described herein; ii) contacting the matrix members with a target; and iii) recording the interaction of the target with each peptide in the matrix, where the collective interactions of the target with the matrix forms a fingerprint of the target with the peptide matrix. In certain embodiments the recording comprises outputting to a printer or display and/or recording to a computer readable medium (e.g., magnetic medium, optical medium, flash memory, etc.). In various embodiments the library is a peptide library as described herein.

Methods are also provided of identifying and/or characterizing a target. The methods typically involve contacting the target with members of a sparse matrix library as described herein; recording a pattern of interaction of the target and the library member(s); iii) comparing the pattern of interaction of the target and the library members to a database previously determined patterns of interaction for one or more known targets; and iv) determining that the target is or is not one or said one or more known targets.

In various embodiments methods are provided of determining whether a member of a sparse matrix competes with the binding of a compound to a target. The methods typically involve i) providing a sparse matrix library as described herein; ii) contacting a target with the members of said library in the presence of a compound, wherein the compound binds an epitope of the target; iii) measuring the level of interaction between library member(s) and the target and between the compound and the target, wherein the increasing interaction of the member(s) and the target and decreasing interaction of the target and the compound indicates that a compound in the matrix competes the binding of the compound to the target through the same or similar or overlapping epitope.

One aspect of the present invention relates to a compound library which comprises a two-dimensional matrix of compounds designed by incorporation of two parameters of the compounds: in a longitudinal direction or axis, the compounds changes the value of a first parameter in a stepwise fashion; and in a latitudinal direction or axis, the compounds changes the value of a second parameter in a stepwise fashion.

In one embodiment, a compound is a polymeric molecule comprising a plurality of monomer units coupled with one by one in a liner fashion. Linear polymeric molecules or liner polymers include, for example, peptides, nucleic acids, and peptide nucleic acids.

In another embodiment, the compound is a peptide. The first and second parameters of the peptides are hydrophobicity and electronic charge respectively.

Another aspect of the present invention relates to a sparse matrix or matrix peptide library wherein the peptide library is a two-dimensional matrix of peptides designed by incorporating two parameters: 1) the level of hydrophobicity in peptides along a longitudinal direction or axis (e.g., from high hydrophobicity to low hydrophobicity); and 2) the level of electricity charge in peptides along a latitudinal direction or axis (e.g., from highly negative charge to highly positive charge).

Another aspect of the present invention relates to a method of designing and generating a peptide library of the present invention.

Another aspect of the present invention relates to a method of identifying a peptide or peptides which specifically binds to a target using the peptide library of the present invention.

Another aspect of the present invention relates to a method of identifying a pattern of interaction (a fingerprint) between a target and a peptide library of the present invention.

Another aspect of the present invention relates to a method of identifying a target to be tested (or an unknown target) or the presence or the properties of the target thereof using the peptide library of the present invention and/or the fingerprint.

Another aspect of the present invention relates to a method of determining a peptide in the peptide library of the present invention which competes the binding of a compound to a target or binds competitively to the same epitope of the target as the compound.

In certain embodiments, this invention expressly excludes nucleic acid libraries (libraries comprising polynucleotides). In certain embodiment, this invention expressly excludes combinatorial libraries and/or libraries based on the modification of compounds with a pre-identified biological activity.

### DEFINITIONS

The term “peptide” as used herein refers to a polymer of amino acid residues typically ranging in length from 2 to about 50 residues. In certain embodiments the peptide ranges in length from about 2, 3, 4, 5, 7, 9, or 10 residues to about 50, 45, 40, 45, 30, 25, 20, or 15 residues. In certain embodiments the peptide ranges in length from about 8, 9, 10, 11, or 12 residues to about 20 or 25 residues. In certain embodiments the amino acid residues comprising the peptide are “L-form” amino acid residues, however, it is recognized that in various embodiments, “D” amino acids can be incorporated into, or form the entire, peptide. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an α-ester, a β-ester, a thioamide, a phosphonamide, a carbomate, a hydroxylate, and the like, see, e.g., Spatola, (1983) Chem. Biochem. Amino Acids and Proteins 7: 267-357, where the amide is replaced with a saturated amine (see, e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

Where the amino acid sequence of a peptide is provided herein, it will be understood to contemplate the retro-sequence, inverse sequence, and retro-inverse sequence. In retro forms, the direction of the sequence is reversed. Thus the amino acid sequence WWWWWEEEE (SEQ ID NO: 1) is the retro sequence of the amino acid sequence EEEEWWWWW (SEQ ID NO:2). In inverse forms, the chirality of the constituent amino acids is reversed (i.e., L form amino acids become D form amino acids and D form amino acids become L form amino acids). In the retro-inverse form (a.k.a. retro-inverso form), both the order and the chirality of the amino acids is reversed.

The term “residue” as used herein refers to natural, synthetic, or modified amino acids. Various amino acid analogues include, but are not limited to 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, n-ethylglycine, n-ethylasparagine, hydroxylysine, allo-hydroxylysine, 3-hydroxyproline, 4-hydroxyproline, isodesmosine, allo-isoleucine, n-methylglycine, sarcosine, n-methylisoleucine, 6-n-methyllysine, n-methylvaline, norvaline, norleucine, ornithine, and the like. These modified amino acids are illustrative and not intended to be limiting.

Peptoids, or N-substituted glycines, are a specific subclass of peptidomimetics. They are closely related to their natural peptide counterparts, but differ chemically in that their side chains are appended to nitrogen atoms along the molecule's backbone, rather than to the α-carbons (as they are in natural amino acids).

The terms “conventional” and “natural” as applied to peptides herein refer to peptides, constructed only from the naturally-occurring amino acids: Ala, Cys, Asp, Glu, Glu, Phe, Gly, His, Ile, Lys, Leu, Met, Asn, Pro, Gln, Arg, Ser, Thr, Val, Trp, and Tyr. A compound of the invention “corresponds” to a natural peptide if it elicits a biological activity related to the biological activity and/or binding specificity of the naturally occurring peptide. The elicited activity may be the same as, greater than or less than that of the natural peptide. In general, such a peptoid will have an essentially corresponding monomer sequence, where a natural amino acid is replaced by an N-substituted glycine derivative, if the N-substituted glycine derivative resembles the original amino acid in hydrophilicity, hydrophobicity, polarity, etc. Thus, for example, the following pairs of peptides would be considered “corresponding”:

(SEQ ID NO: 3)

Ia. Asp-Arg-Val-Tyr-Ile-His-Pro-Phe
(Angiotensin II)
and

(SEQ ID NO: 4)

Ib. Asp-Arg-Val*-Tyr-Ile*-His-Pro-Phe;

(SEQ ID NO: 5)

IIa. Arg-Pro-Pro-Gly-Phe-Ser-Pro-Phe-Arg
(Bradykinin)
and

(SEQ ID NO: 6)

IIb: Arg-Pro-Pro-Gly-Phe*-Ser*-Pro-Phe*-Arg;

(SEQ ID NO: 7)

IIIa: Gly-Gly-Phe-Met-Thr-Ser-Glu-Lys-Ser-Gln-Thr-
Pro-Leu-Val-Thr (β-Endorphin);
and

(SEQ ID NO: 8)

IIIb: Gly-Gly-Phe*-Met-Ser*-Ser-Glu-Lys*-Ser*Gln-
Ser*-Pro-Leu-Val*-Thr.

In these examples, “Val*” refers to N-(prop-2-yl)glycine, “Phe*” refers to N-benzylglycine, “Ser*” refers to N-(2-hydroxyethyl)glycine, “Leu*” refers to N-(2-methylprop-1-yl)glycine, and “Ile*” refers to N-(1-methylprop-1-yl)glycine. The correspondence need not be exact: for example, N-(2-hydroxyethyl)glycine may substitute for Ser, Thr, Cys, and Met; N-(2-methylprop-1-yl)glycine may substitute for Val, Leu, and Ile. Note in IIIa and IIIb above that Ser* is used to substitute for Thr and Ser, despite the structural differences: the sidechain in Ser* is one methylene group longer than that of Ser, and differs from Thr in the site of hydroxy-substitution. In general, one may use an N-hydroxyalkyl-substituted glycine to substitute for any polar amino acid, an N-benzyl- or N-aralkyl-substituted glycine to replace any aromatic amino acid (e.g., Phe, Trp, etc.), an N-alkyl-substituted glycine such as N-butylglycine to replace any nonpolar amino acid (e.g., Leu, Val, Ile, etc.), and an N-(aminoalkyl)glycine derivative to replace any basic polar amino acid (e.g., Lys and Arg).

A “naive library of compounds” refers to a library of compounds that comprise modifications of a compound that is not naturally occurring and/or that has no known biological activity and/or biological binding affinity and/or specificity.

The term “systematically varied” when used with respect to a property that is systematically varied in a sparse matrix refers to a property that is selected in the design of the matrix so that compounds comprising the matrix are chosen to provide a plurality of different values for that property. In certain embodiments the compounds are chosen to provide a plurality of different values for that property where the values span substantially uniformly or non-uniformly a particular predetermined range.

The terms “matrix” and “library” are used interchangeably to refer to a collection of compounds (e.g., nucleic acids, peptides, carbohydrates, etc.). In certain embodiments the members of the matrix are provided attached to a solid support.

The term “specifically bind” when referring to the interaction of a compound in a sparse matrix library and a target refers to a binding reaction that is determinative of the presence one or the other member of the binding pair (compound or target) in the presence of a heterogeneous population of molecules (e.g., proteins and other biologics), or in a heterogenous population of cells (where the target is a cell) or in a heterogenous population of bacteria where the target is a bacterium, and so forth. In certain instances, the specific binding can simply be preferential binding where sufficient preference is given to the target so that the target can be preferentially differentiated from the mixed population. In certain embodiments specific or preferential binding is with an avidity at least 1.2 fold, preferably at least 1.5 fold, more preferably at least 2-fold, 4-fold, 8-fold, or at least 10-fold greater for the target than for other moities accessable to the compound.

The term “oligosaccharide” refers to a saccharide polymer containing a small number (typically two to about 30, preferably 2 to about 20, more preferably 2 to about 15, or 13, or 12, or 10, or 9, or 6) of component sugars. Oligosaccharides include naturally occurring sugars as well as modified sugars.

The terms “nucleic acid” or “oligonucleotide” refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments. Preferred nucleic acids if used in this invention range from about 5 nucleotides to about 500 nucleotides, preferably from about 5 nucleotides to about 100 nucleotides, more preferably from about 5nucleotides to about 50 nucleotides, and most preferably from about 5 nucleotides to about 10, 15, 20, 30, 40, or 50 nucleotides in length.

A “compound matrix” refers to a collection of compounds that are systematically varied with respect to two or more properties. The “dimensionality” of the matrix refers to the number of parameters that are systematically varied. Thus, for example, a 2 dimensional matrix systematically varies two properties (e.g., hydrophobicity and charge), a 3-dimensional matrix systematically varies three properties (e.g., hydrophobicity, charge, and periodicity), a 4-dimensional matrix systematically varies four different properties, and so forth.

The phrase “compounds having essentially the same value of the first property” refers to compounds where the variation of the first property is less than about 25%, preferably less than about 20% or 15%, more preferably less than about 10% or 5% or 2% or 1%.

### BRIEF DESCRIPTION OF THE DRAWINGS

FIGS. 1A and 1B schematically illustrate embodiments, of a sparse matrix as described herein. FIG. 1A schematically illustrates a 2-dimensional matrix, while FIG. 1B schematically illustrates a four-dimensional matrix.

FIG. 2 illustrates a schematic figure of an m×n peptide matrix.

FIG. 3 illustrates the design of an illustrative pilot library. Sequences (top) are arranged in a 2D matrix to provide extensive variation in the parameters of charge (bottom left, plot shows formal charge at pH 7.0) and hydrophobicity (see, e.g., Fauchere and Pliska (1983) Eur. J. Med. Chem., 18: 369-375) (bottom right). X and Y axes represent matrix position as defined at top; Z axes show formal charge (amu) and hydrophobicity (arbitrary units), respectively. Peptide sequences: A1 (SEQ ID NO:9), A2 (SEQ ID NO: 10), A3 (SEQ ID NO:11), A4 (SEQ ID NO:12), A5 (SEQ ID NO:13), A6 (SEQ ID NO: 14), B1 (SEQ ID NO:15), B2 (SEQ ID NO:16), B3 (SEQ ID NO: 17), B4 (SEQ ID NO:18), B5 (SEQ ID NO:19), B6 (SEQ ID NO:20), C1 (SEQ ID NO:21), C2 (SEQ ID NO:22), C3 (SEQ ID NO:23), C4 (SEQ ID NO:24), C5 (SEQ ID NO:25), C6 (SEQ ID NO:26), D1 (SEQ ID NO:27), D2 (SEQ ID NO:28), D3 (SEQ ID NO:29), D4 (SEQ ID NO:30), D5 (SEQ ID NO:31), D6 (SEQ ID NO:32), E1 (SEQ ID NO:33), E2 (SEQ ID NO:34), E3 (SEQ ID NO:35), E4 (SEQ ID NO:36), E5 (SEQ ID NO:37), E6 (SEQ ID NO:38), F1 (SEQ ID NO:39), F2 (SEQ ID NO:40), F3 (SEQ ID NO:41), F4 (SEQ ID NO:42), F5 (SEQ ID NO:43) (SEQ ID NO:44), and F6 (SEQ ID NO:45).

FIGS. 4A and 4B illustrate binding of the refined libraries (sequences given in FIG. 3) to Staphylococcus aureus strain AM1. FIG. 4A: Fluorescence images, collected using identical microscope and camera settings. Peptides are arranged as in FIG. 3. FIG. 4B: Quantitation of fluorescence images. Binding profile shows the relative intensity of staining for each peptide in the matrix. X and Y axes represent matrix position as defined in FIG. 3; Z axis shows fluorescence intensity from stained cells as defined in the Examples (arbitrary units)

FIG. 5 shows “peptide fingerprints” of various bacteria and other cells. Cells were immobilized, exposed to the peptides from the initial pilot library, and imaged with image quantitation as described in the Examples. Relative levels of peptide binding (as indicated by the intensity of fluorescent staining) are shown. The relative levels of binding vary replicably from bacterium to bacterium. Note especially the comparison of the two M. xanthus strains: while the overall binding profile is similar, the lower half of the library contains peptides that bind to the wild type but not the exopolysaccharide-deficient strain, thus allowing these strains to be distinguished on the basis of their peptide binding patterns. X and Y axes represent matrix position as defined in FIG. 2; Z axes show fluorescence intensity from stained cells as defined in Methods (arbitrary units).

FIG. 6: (Top Row) Pilot library binding profiles of selected eukaryotic cells (C. albicans and Chinese Hamster Ovary (CHO) cells) and one bioinorganic surface (Human Tooth). X and Y axes represent matrix position as defined in FIG. 2; Z axes show fluorescence intensity from stained cells as defined in Methods (arbitrary units). (Bottom Row) Fluorescence images showing binding of peptide 3A from the initial pilot library to both C. albicans and CHO cells, and binding of peptide E4 to a sectioned human tooth. In the case of the tooth section, clear fluorescent staining is visible in the Enamel (E) as well as on the enamel side of the Dentin-Enamel Junction (DEJ). No significant staining was observed to Dentin (D) or in other non-enamel tissues (not shown).

FIGS. 7A and 7B show sequences of peptides from the refined libraries. FIG. 7A: Top: Sequences of peptides from the in-plane refined library, Bottom: Fluorescence images showing the binding of labeled peptides (as indicated) to immobilized S. aureus cells. Peptides: A1 (SEQ ID NO:46), A2 (SEQ ID NO:47), A3 (SEQ ID NO:48), A4 (SEQ ID NO:49), B1 (SEQ ID NO:50), B2 (SEQ ID NO:51), B3 (SEQ ID NO:52), B4 (SEQ ID NO:53), C1 (SEQ ID NO:54), C2 (SEQ ID NO:55), C3 (SEQ ID NO:56), C4 (SEQ ID NO:57), D1 (SEQ ID NO:58), D2 (SEQ ID NO:59), D3 (SEQ ID NO:60), D4 (SEQ ID NO:61). FIG. 7B: Top: Sequences of peptides from the orthogonal refined library, Bottom: Fluorescence images showing the binding of labeled peptides (as indicated) to immobilized S. aureus cells. Peptides: A1 (SEQ ID NO:62), A2 (SEQ ID NO:63), A3 (SEQ ID NO:64), A4 (SEQ ID NO:65), B1 (SEQ ID NO:66), B2 (SEQ ID NO:67), B3 (SEQ ID NO:68), B4 (SEQ ID NO:69), C1 (SEQ ID NO:70), C2 (SEQ ID NO:71), C3 (SEQ ID NO:72), C4 (SEQ ID NO:73), D1 (SEQ ID NO:74), D2 (SEQ ID NO:75), D3 (SEQ ID NO:76), D4 (SEQ ID NO:77).

FIG. 8: Specificities of representative binding peptides. A) Fluorescence images were recorded and quantitated for peptide bound to various bacterial species as described in Methods. Top row: binding of peptides 3C and 4D from the original pilot library, showing significant binding to several strains. Middle row: binding of peptide 1B from the In-Plane refined library, showing binding to S. aureus and M. luteus. Bottom row: binding of peptide 3A from the In-Plane refined library showing binding to S. aureus only.

### DETAILED DESCRIPTION

### I. Sparse Matrix Libraries

In certain embodiments, this invention pertains to the creation of “sparse matrix” libraries of compounds. The sparse matrix libraries typically span an n-dimensional parameter (or property) space at extremely sparse intervals and thereby provide a relatively small library of compounds (e.g., a library of about 1,000 or fewer, preferably about 500 or fewer, more preferably about 200 or fewer, and most preferably about 100 or fewer different compounds) that can be used to screen the compound parameter space for one or more desired physical, chemical, or biological properties (e.g., binding specificity, binding avidity, toxicity, solubility, mobility, cell permeability, serum half-life, biocompatibility, etc.). Unlike screening systems where known proteins (e.g., certain physiologically active proteins) are systematically mutated and screened for altered activity, the sparse matrix libraries can be based on “naive” peptide sequences, i.e., sequences with no known or predetermined biological activity.

Having relatively few members, the sparse matrix libraries can be rapidly fabricated and screened to identify members with the desired properties/activity. These initial “hits” (positive-scoring compounds) provide information about properties varied across the parameter space of the sparse matrix library and provide a basis for preparing small refined libraries that to screen the same parameter space at higher resolution in the region around the positive-scoring compounds, or with respect to other parameters to further optimize the compounds for the desired properties.

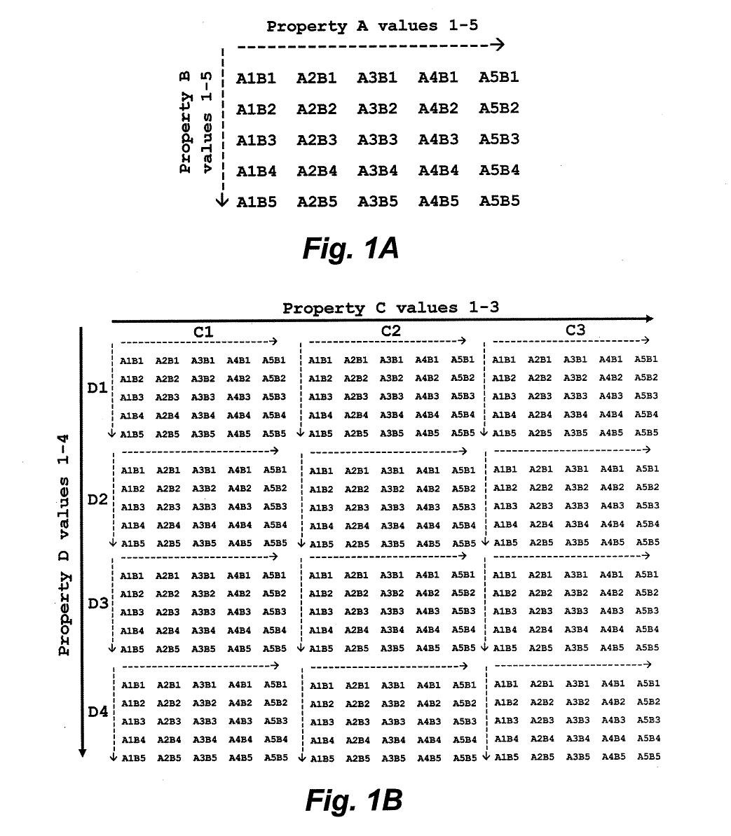

By way of illustration, in certain embodiments, the creation of sparse compound libraries (e.g., polysaccharides, lipid polymers, peptides, peptoids, etc.) are contemplated that vary in, for example, two properties (i.e., the libraries span a 2-dimensional parameter space), a first property and a second property. For example, a peptide/peptoid sparse matrix library can be prepared, where the library members vary systematically with respect to, for example, hydrophobicity (first property/parameter), and charge (second property/parameter). This is schematically illustrated in FIG. 1A which represents a sparse matrix characterized by two properties “A” and “B” that are systematically varied. In this illustration, property “A”, hydrophobicity for example, varies along each row and has one of five different possible values (A1 through A5). Property “B”, charge for example, varies along each column and also has one of five different possible values (designated B1 through B5). Thus, each member of the library is characterized by a particular hydrophobicity and charge (e.g., A1B1, A2B3, etc.).

The total range in the values of each property and the number of compounds in the library determines the “sparseness” (resolution) of the matrix. In certain embodiments each property varies between a high and a low value (e.g., large negative charge to large positive charge, from hydrophobic to hydrophilic, etc.) and the intervening compounds can be chosen to provide values of the properties that vary in a linear/stepwise manner. The variation, however need not be linear. For example, matrix members can be chosen so that values for various properties cluster around particular points, vary in a logarithmic or exponential manner, or merely span a range in whatever manner is convenient.

While FIG. 1A illustrates a two-dimensional matrix, it will be recognized that sparse matrices of this invention are not limited to two-dimensional matrices. To the contrary, in various embodiments, N-dimensional matrices are contemplated where n is the number of properties that are systematically varied in the matrix. In various embodiments N ranges from 2 up to about 100, more preferably from 2 up to about 80, 60, 50, 40, 30, 20, or 10, and in certain embodiments, from 2 up to 3, 4, 5, 6, 7, 8, 9, or 10.

A four-dimensional matrix is illustrated in FIG. 1B. In this illustration the sparse matrix systematically varies in four properties A, B, C, and D. Properties A and B (e.g., hydrophobicity and charge, respectively) each have five different possible values as described above. For each of these compounds, a third property C (e.g., length) is varied systematically and, as shown in the illustration, C has three possible values (C1, C2, and C3). A fourth property D (e.g., periodicity) is also varied and has four different possible values.

Thus, in certain embodiments, a sparse matrix is contemplated where the matrix has dimensionality D, where D is the number of different compound properties systematically varied in the library. Compounds comprising the library are characterized by these systematically varied properties (e.g., P1, P2, P3, P4, . . . through PD). Compounds are selected so that each systematically varied property, e.g., P1, P2, P3, PD, etc., is present at a multiplicity of different values (e.g., from 2 different values up to about 100, or up to about 80, or up to about 60, or up to about 50, or up to about 40, or up to about 30, or up to about 20, or up to about 10, or up to about 10, or 9, or 8, or 8, or 6, or 5, or 4, or 3, different values).

Thus, for example, each member of the library can be uniquely identified for example, as C_{P1i, P2j, PDx}, where each subscript is associated with a particular value of each property systematically varied property. This nomenclature can be shortened so that the compound is represented as C_{i, j, k} where each subscript i, j, k (up to the total number of properties) is associated with a particular value of a property. The range of i, j, k and so forth represents the number of possible different values that the property can take in the matrix. Thus, if subscript i represents hydrophobicity, and i ranges up to 10, then the compounds comprising the sparse matrix have essentially 10 different possible hydrophobicities, and each value of i is associated with a different hydrophobicity. Thus all compounds designated subscript i have substantially the same hydrophobicity although they may vary with respect to other properties. A second property (e.g., charge) can be represented by the second subscript j. If j ranges up to 6 the compounds comprising the sparse matrix have essentially 6 different possible charges (charge states) and each value of j (e.g., 1-6) is associated with a different charge state. All compounds designated with the same j value have substantially the same charge state. This can, of course be extended to any number of dimensions where each additional dimension represents an additional systematically varied property and each member along that dimension can be represented by a new subscript.

Accordingly, one aspect of the present invention relates to a compound library that comprises a two-dimensional matrix of compounds designed by incorporation of two parameters of the compounds: in a “longitudinal” direction or axis, the compounds change the value of a first parameter in a stepwise fashion; and in a “latitudinal” direction or axis, the compounds change the value of a second parameter in a stepwise fashion. In another embodiment, the compound is a peptide and the two parameters of the peptide are hydrophobicity and electronic charge respectively.

One aspect of the present invention relates to a matrix peptide library wherein the peptide library is a two-dimensional matrix of peptides designed by incorporating two parameters: 1) the level of hydrophobicity in peptides along a longitudinal direction or axis (e.g., from high hydrophobicity to low hydrophobicity); and 2) the level of electricity charge in peptides along a latitudinal direction or axis (e.g., from highly negative charge to highly positive charge).

In one embodiment, the two-dimensional matrix comprises a plurality or an array of peptides wherein the peptides along the longitudinal direction (e.g., along the rows of the matrix) vary in a first parameter (e.g., hydrophobicity) in a liner, stepwise fashion; the peptide along latitudinal direction (e.g., along the columns of the matrix) vary in a second parameter (e.g., electric charge) in a liner, stepwise fashion.

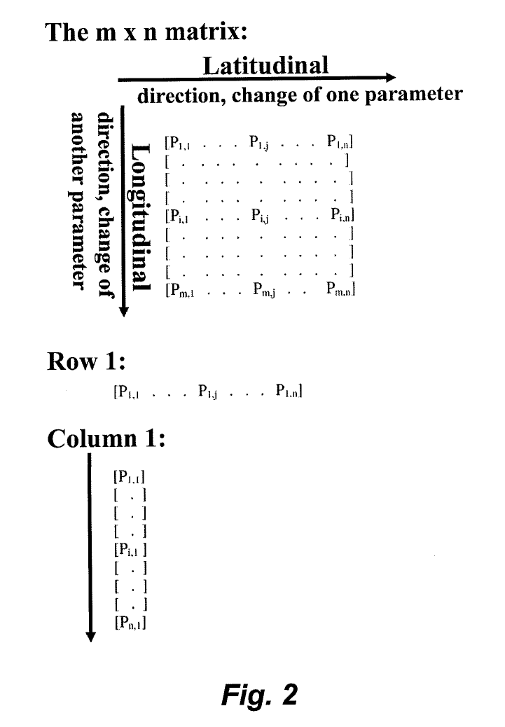

In another embodiment, the two-dimensional matrix of peptides is an m×n matrix as shown in FIG. 2. The library comprises an order or size of m×n peptides having m rows of peptides (each row comprises n peptides) and n columns of peptides (each column comprises m peptides), wherein 2≦m≦100, 2≦n≦100. A peptide in the matrix is denoted as p_{i,j} and assigned with a first and second parameter (e.g., hydrophobicity and charge), whereas 1≦i≦m and 1≦j≦n.

In certain embodiments the characteristics of a peptide library having an m×n matrix can be described as follows:

1) in each given row, e.g., row i (p_{i,1}, p_{i,2}, . . . p_{i,n}), the values of the first parameter (for example, the hydrophobicity) for all the peptides in the row are kept substantially the same or similar whereas the values of the second parameter (for example, the charge) change latitudinally in stepwise manner (for example, from highly positive to highly negative);

2) in each given column, e.g., column j (p_{1,j}, p_{2,j}, . . . p_{m,j}), the values of the second parameter (for example, the charge) of all the peptides in the column are kept substantially the same or similar whereas the values of the first parameter (for example, the hydrophobicity) change longitudinally in stepwise manner (e.g., from highly hydrophobic to highly hydrophilic);

In another embodiment, the peptides at the four corners of the matrix (p_{i,1}, p_{m,1}, p_{1,n} and p_{m,n}) possess the high and/or low end values for each parameter.

In another embodiment, the order or the size of the m×n matrix is ≦about 10,000, ≦about 5,000, ≦about 4,000, ≦about 3,000, ≦about 2,000, ≦about 1,000, ≦about 900, ≦about 800, ≦about 700, ≦about 600, ≦about 500, ≦about 400, ≦about 300, ≦about 200, ≦about 100, ≦about 90, ≦about 80, ≦about 70, ≦about 60, ≦about 50, ≦about 40, ≦about 30, ≦about 20, or ≦about 10 peptides.

In another embodiment, in the m×n matrix, 2≦m≦100, 2≦m≦90, 2≦m≦80, 2≦m≦70, 2≦m≦60, 2≦m≦50, 2≦m≦40, 2≦m≦30, 2≦m≦20, or 2≦m≦10. In addition, m may be 10, 9, 8, 7, 6, 5, 4, 3, or 2.

In another embodiment, 2≦n≦100, 2≦n≦90, 2≦n≦80, 2≦n≦70, 2≦n≦60, 2≦n≦50, 2≦n≦40, 2≦n≦30, 2≦n≦20, or 2≦n≦10. In addition, n may be 10, 9, 8, 7, 6, 5, 4, 3, or 2.

Any of a number of different compounds can be used as members of a sparse matrix. In various embodiments polymeric molecules are used, and in certain embodiments, biological polymeric molecules are preferred. The polymeric molecules can be branched or straight chain polymers and comprise naturally-occurring and/or synthetic subunits. In various embodiments suitable polymeric molecules include peptides (including natural peptides or modified peptides (e.g., peptoids)), polysaccharides, glycopeptides, polymeric lipids, nucleic acids (including modified nucleic acids), mixed amino acid/nucleotide polymers, and so forth.

In various embodiments the length of the polymeric compounds ranges from 2 up to about 50 monomers, more typically from about 2 up to about 50, 40, 30, 25, 20, 18, 15, 13, 12, 10, 8, or 6 monomers.

In certain embodiments, embodiment, the polymeric compounds (e.g., peptides comprising the matrix) have a desired length in term of the number of monomers (e.g., amino acids in a peptide). In particular, the compounds (e.g., peptides) have a length of χ monomers (e.g., aminoacids),wherein χ is 4, 5, 6, 7, 8, 9, 10, 11, 12, 13, 14, 15, 16, 17, 18, 19, 20, 30, 40, or 50. Preferably, χ is an integer between 4 to 20.

In various embodiments, members of a sparse matrix library are based on “naive” compounds and/or sequences (e.g., compounds and/or sequences for which no binding affinity or other biological activity is known and/or are not found in nature). The sparse matrix libraries can be constructed so that they systematically vary, e.g., as described above with respect to various properties. Essentially any physical property of the compounds can be varied. Such properties include, but are not limited to hydrophobicity, charge, length, molecular weight, stokes radius, periodicity, van der Waals radius, conformational flexibility, aromaticity, aliphatic index, helix-forming potential in aqueous solution, helix-forming potential in hydrophobic environments, dipole moment, sheet forming potential, polarizability, and the like and includes any ratio or other metric computed from any of these parameters or combinations thereof.

While the sparse matrices are illustrated in a planar manner in FIGS. 1A, 1B, and 2, these figures are intended to be illustrative of the composition of the library, and do not require that the compounds comprising the matrix be so disposed. To the contrary, while certain embodiments contemplate a physical arrangement of the various members of a sparse matrix library disposed on a substrate, or in wells (e.g., in a microtiter plate) or on pins or other features that impart a spatial orientation, in other embodiments, the members of the sparse matrix library are simply contained individually or in combination in one or more containers and no particular spatial orientation is imparted.

### II. Matrix Design and Fabrication

In certain embodiments methods are provided for designing and generating a peptide library of the present invention. In certain embodiments the methods involve providing the length of the peptides to be synthesized; providing the first parameter(s), second parameters (and other parameters, where desired) of the peptides; providing the scope of the parameters to be designed or screened; providing an approximate step size for changing the parameters; and providing the size or the number of peptides in the library.

In one embodiment, the sparse matrix library (e.g., an m×n matrix peptide library) is designed by considering 1) a desired size for the peptides to be synthesized, 2) a desired step size for the incremental change of a parameter or parameters, and 3) a desired spectrum or scope of the parameters to be screened (e.g., a high end value and low end value of two parameters that can be varied in a stepwise fashion, e.g., through two (or more) directions/dimensions (e.g., longitudinally and latitudinally) in the matrix). In certain embodiments the corners of the matrix (p_{1,1}, p_{1,n}, p_{m,1}, p_{m,n}) coincide with high end and low end values of the chosen parameters.

For an example, an m×n matrix peptide library can be designed through the steps of 1) providing a first scope of a first parameter along the longitudinal direction or the row direction (e.g., from the end value +1 to the end value −1 for the charge (C)) to be screened; 2) providing a second scope of a second parameter along the latitudinal direction or the column direction (e.g., from end value +0.2 to the end value −0.2 for the hydrophobicity (H)); 3) providing an approximate first step size for the first parameter (e.g., 0.1) and an approximate second step size for the second parameter (e.g., 0.05), 4) calculating the size of the matrix (e.g., in terms of m×n, m=1+(1−(−1))/0.1=21, n=1+(0.2−(−0.2))/0.05=9), 5) choosing a peptide length and four peptides for the four corners of the matrix such that the properties of the peptides at four corners of the matrix substantially coincide with the end values of the parameters (e.g., p_{1,1} having C+1 and H+0.2, p_{21,1} having C−1 and H+0.2 p_{1,9} having C+1 and H−0.2, p_{21,9} having C−1 and H−0.2) and the step size can be achieved by changing, substituting or alternating amino acid residues (or subsets of amino acid residues) from the four peptides; and 6) changing, substituting or alternating amino acid residues from at least one peptide from the four peptides at four corners such that the step size for each parameter can be reached.

For another example, an m×n matrix peptide library can be designed through the steps of 1) providing a peptide length (e.g., having 9 amino acid residues), 2) providing a high end value and a low end value for a first parameter (e.g., C+1 to C−1) and a high end value and a low end value for a second parameter (e.g., H+0.2 to H−0.2), 3), 3) providing an approximate first step size for the first parameter and an approximate second step size for the second parameter, 4) designing at least one peptide having the peptide length and the end values for both parameter (e.g., C+1 and H+0.2, or C−1 and H+0.2 or C+1 and H−0.2 or C−1 and H−0.2), 5) calculating the size of the matrix such that the scopes of both parameters can be screened in a stepwise manner according to predetermined step sizes; and 6) changing, substituting or alternating amino acid residues or subset thereof from at least one peptide such that the stepwise change for each parameter can be reached. In another example, in any given peptide length, four peptides which reside at the four corners of the matrix and possess the end values of the parameters (e.g., p_{1,1} having C+1 and H+0.2, p_{m,1} having C−1 and H+0.2 p_{1,n} having C+1 and H−0.2, p_{m,n} having C−1 and H−0.2) are provided.

For another example, an m×n matrix peptide library can be designed through the steps of 1) providing a peptide length (e.g., having 9 amino acid residues), 2) providing a desired size of the matrix, 3) providing a high end value or a low end value for a first parameter (e.g., C+1 to C−1), 4) providing a high end value or a low end value for a second parameter (e.g., H+0.2 to H−0.2), 5) providing an approximately first step size for the first parameter and an approximately second step size for the second parameter, 6) providing a first peptide having the peptide length and the end values for both parameter (e.g., C+1 and H+0.2, or C−1 and H+0.2 or C+1 and H−0.2 or C−1 and H−0.2) and placing the first peptide at any position in the matrix, and 7) changing, substituting or alternating amino acid residues or subset thereof from the first peptide such that the stepwise change for each parameter can be reached longitudinally and latitudinally. In a preferred embodiment, in any given peptide length, the first peptide resides at one of the four corners of the matrix and possess the end values of the parameters (e.g., p_{1,1} having C+1 and H+0.2, p_{m,1} having C−1 and H+0.2 p_{1,n} having C+1 and H−0.2,p_{m,n} having C−1 and H−0.2.) In another preferred embodiment, the first peptide resides at any position of the matrix (e.g., p_{i,j},) and possess the end values of the parameters. Along the longitudinal direction, the values of first parameter change (decrease or increase) in other peptides in the column in a stepwise fashion starting from the first peptide; along the latitudinal direction, the values of the second parameter changes (decrease or increase) in other peptides in the row in a stepwise fashion starting from the first peptide.

In one illustrative example, the matrix library is designed by choosing a desired size (the desired number of amino acid residues) for the peptides to be synthesized and two parameters that can be varied in a stepwise fashion by alternating subsets of amino acid residues. The amino acid residue positions along the length of a peptide are assigned to a amino acid(s) having known values for the given parameter (see, e.g., Table 1, below, for the charge and hydrophobicity value for each amino acid). A first peptide, e.g., at one corner of the matrix, is designed to having the end values for both parameter and therefore comprises amino acid residues in their respective position that show these values. For example, in WWKHWWHRW (SEQ ID NO:78), a 9-mer peptide, amino acid W (Try having a hydrophobicity value of 2.25) is assigned to position 1, 2, 5, 6, and 9 for the first parameter (e.g., hydrophobicity); and amino acid K (Lys), H (His) and R (Arg) having a charge value of +1 respectively are assigned to position 2, 4, 7, and 8 for the second parameter (e.g., charge). The amino acid residue positions assigned to the first parameter are varied in that parameter in a linear, stepwise fashion latitudinally or in the columns of the matrix, and the positions assigned to the second parameter are varied in that parameter in a linear, stepwise fashion longitudinally or in the rows of the matrix.

In this example, the size for a pilot library is determined by the length of the peptide and the distribution of positions reserved for each parameter. In the case of discrete parameters, each possible value of the chosen parameter may be represented in one dimension of the matrix: for example, in the case of peptides that are 9 amino acid residues consisting of two hydrophobic positions, followed by two charged positions, followed by two hydrophobic positions, followed by two charged positions, followed by a carboxy-terminal hydrophobic position, there are four charged positions representing a total of 9 possible discrete charge states (when charge is considered to be a discrete parameter with values of −1, 0, or 1). There is no formal constraint on the number of values chosen for continuous parameters, but in the case of matrices that contain one discrete and one continuous parameter, in order to maximize the information content of the resultant datasets, the number of values chosen for the continuous parameter can be selected to reflect the number of values chosen for the discrete parameter (for example, see the following tables of optimal matrix dimensions for matrices designed using parameters of hydrophobicity and charge).

TABLE 1
The value of hydrophobicity and charge for each amino acid.

| Three- | One- | Hydrophobicity | Henderson- |
|---|---|---|---|
| letter | letter | (Fauchere and | Hasselbalch |
| code | code | Pliska, 1983) | charge |
| Gly | G | 0 | 0 |
| Ala | A | 0.31 | 0 |
| Val | V | 1.22 | 0 |
| Leu | L | 1.7 | 0 |
| Ile | I | 1.8 | 0 |
| Ser | S | 0.04 | 0 |
| Thr | T | 0.26 | 0 |
| Cys | C | 1.54 | −0.03 |
| Pro | P | 0.72 | 0 |
| Phe | F | 1.79 | 0 |
| Tyr | Y | 0.96 | 0 |
| Trp | W | 2.25 | 0 |
| His | H | 0.13 | 0.24 |
| Asp | D | −0.77 | −1 |
| Asn | N | −0.6 | 0 |
| Glu | E | −0.64 | −1 |
| Gln | Q | −0.22 | 0 |
| Met | M | 1.23 | 0 |
| Lys | K | −0.99 | 1 |
| Arg | R | −1.01 | 1 |

See also Fauchere and Pliska (1983) Eur. J. Med. Chem., 18: 369.

The amino acid residue positions within a peptide sequence can be arranged in many different ways, as long as the corners of the matrix coincide with a desired end values of the chosen parameter. For example, a first peptide at a first corner (p_{1,1}) is highly hydrophobic, corresponding to tryptophans in all assigned hydrophobic positions, and highly negatively charged, corresponding to having aspartic acid residues in all assigned hydrophilic positions. A second peptide at a second corner (p_{1,n}) is highly hydrophobic (Trp in all hydrophobic positions) and highly positively charged, corresponding to having arginines in all assigned hydrophilic positions. A third peptide at a third corner (p_{m,1}) is weakly hydrophobic, corresponding to having alanines in all assigned hydrophobic positions and highly negatively charged (Asp in all assigned hydrophilic positions). A fourth peptide at a fourth corner (p_{m,n}) is weakly hydrophobic (Ala in all assigned hydrophobic positions) and highly positively charged (Arg in all assigned hydrophilic positions). In certain embodiments the location of the assigned positions within the sequence remain constant within a single library, but many libraries are possible that have the same combination of parameters and different arrangements of assigned positions. In another example, where the parameters are charge and hydrophobicity and each peptide consists of five hydrophobic amino acids and four hydrophilic amino acids, potential libraries may be defined by the corners, as described above, with several possible examples given in Table 2.

TABLE 2

Illustrates certain arrangements of amino acids that can be
used to generate m × n libraries of 9-residue peptides varying
in hydrophobicity and charge with five hydrophobic positions
and four hydrophilic positions. Sequences are arranged
according to the notation described in FIG. 2 with p_{1,1}
representing the most hydrophobic and most negatively charged
sequence, p_{1,n} representing the most hydrophobic, most
positively charged sequence, p_{m,1} representing the least
hydrophobic, most negatively charged sequence, and p_{m,n}
representing the least hydrophobic, most positively charged
sequence. These sequences have equivalent physicochemical
properties, but vary in the specific arrangement of their
component amino acid residues.

| | | | |
|---|---|---|---|
| (p_{1,1}) | (p_{1,n}) | (p_{m,1}) | (p_{m,n}) |

| | | | | |
|---|---|---|---|---|
| 1: | WWWWWEEEE | WWWWWRRRR | AAAAAEEEE | AAAAARRRR |
| | (SEQ ID NO: 79) | (SEQ ID NO: 80) | (SEQ ID NO: 81) | (SEQ ID NO: 82) |
| 2: | WWWWEWEEE | WWWWRWRRR | AAAAEAEEE | AAAARARRR |
| | (SEQ ID NO: 83) | (SEQ ID NO: 84) | (SEQ ID NO: 85) | (SEQ ID NO: 86) |
| 3: | WWWEWWEEE | WWWRWWRRR | AAAEAAEEE | AAARAARRR |
| | (SEQ ID NO: 87) | (SEQ ID NO: 88) | (SEQ ID NO: 89) | (SEQ ID NO: 90) |
| 4: | WWEWWWEEE | WWRWWWRRR | AAEAAAEEE | AARAAARRR |
| | (SEQ ID NO: 91) | (SEQ ID NO: 92) | (SEQ ID NO: 93) | (SEQ ID NO: 94) |
| 5: | WEWWWWEEE | WRWWWWRRR | AEAAAAEEE | ARAAAARRR |
| | (SEQ ID NO: 95) | (SEQ ID NO: 96) | (SEQ ID NO: 97) | (SEQ ID NO: 98) |
| 6: | EWWWWWEEE | RWWWWWRRR | EAAAAAEEE | RAAAAARRR |
| | (SEQ ID NO: 99) | (SEQ ID NO: 100) | (SEQ ID NO: 101) | (SEQ ID NO: 102) |
| 7: | EWWWWEWEE | RWWWWRWRR | EAAAAEAEE | RAAAARARR |
| | (SEQ ID NO: 103) | (SEQ ID NO: 104) | (SEQ ID NO: 105) | (SEQ ID NO: 106) |
| 8: | EWWWEWWEE | RWWWRWWRR | EAAAEAAEE | RAAARAARR |
| | (SEQ ID NO: 107) | (SEQ ID NO: 108) | (SEQ ID NO: 109) | (SEQ ID NO: 110) |
| 9: | EWWEWWWEE | RWWRWWWRR | EAAEAAAEE | RAARAAARR |
| | (SEQ ID NO: 111) | (SEQ ID NO: 112) | (SEQ ID NO: 113) | (SEQ ID NO: 114) |
| 10: | EWEWWWWEE | RWRWWWWRR | EAEAAAAEE | RARAAAARR |
| | (SEQ ID NO: 115) | (SEQ ID NO: 116) | (SEQ ID NO: 117) | (SEQ ID NO: 118) |
| 11: | EEWWWWWEE | RRWWWWWRR | EEAAAAAEE | RRAAAAARR |
| | (SEQ ID NO: 119) | (SEQ ID NO: 120) | (SEQ ID NO: 121) | (SEQ ID NO: 122) |
| 12: | EEWWWWEWE | RRWWWWRWR | EEAAAAEAE | RRAAAARAR |
| | (SEQ ID NO: 123) | (SEQ ID NO: 124) | (SEQ ID NO: 125) | (SEQ ID NO: 126) |
| 13: | EEWWWEWWE | RRWWWRWWR | EEAAAEAAE | RRAAARAAR |
| | (SEQ ID NO: 127) | (SEQ ID NO: 128) | (SEQ ID NO: 129) | (SEQ ID NO: 130) |
| 14: | EEWWEWWWE | RRWWRWWWR | EEAAEAAAE | RRAARAAAR |
| | (SEQ ID NO: 131) | (SEQ ID NO: 132) | (SEQ ID NO: 133) | (SEQ ID NO: 134) |
| 15: | EEWEWWWWE | RRWRWWWWR | EEAEAAAAE | RRARAAAAR |
| | (SEQ ID NO: 135) | (SEQ ID NO: 136) | (SEQ ID NO: 137) | (SEQ ID NO: 138) |
| 16: | EEEWWWWWE | RRRWWWWWR | EEEAAAAAE | RRRAAAAAR |
| | (SEQ ID NO: 139) | (SEQ ID NO: 140) | (SEQ ID NO: 141) | (SEQ ID NO: 142) |
| 17: | EEEWWWWEW | RRRWWWWRW | EEEAAAAEA | RRRAAAARA |
| | (SEQ ID NO: 143) | (SEQ ID NO: 144) | (SEQ ID NO: 145) | (SEQ ID NO: 146) |
| 18: | EEEWWWEWW | RRRWWWRWW | EEEAAAEAA | RRRAAARAA |
| | (SEQ ID NO: 147) | (SEQ ID NO: 148) | (SEQ ID NO: 149) | (SEQ ID NO: 150) |
| 19: | EEEWWEWWW | RRRWWRWWW | EEEAAEAAA | RRRAARAAA |
| | (SEQ ID NO: 151) | (SEQ ID NO: 152) | (SEQ ID NO: 153) | (SEQ ID NO: 154) |
| 20: | EEEEWWWWW | RRRRWWWWW | EEEEAAAAA | RRRRAAAAA |
| | (SEQ ID NO: 155) | (SEQ ID NO: 156) | (SEQ ID NO: 157) | (SEQ ID NO: 158) |
| 21: | WWWWEEWEE | WWWWRRWRR | AAAAEEAEE | AAAARRARR |
| | (SEQ ID NO: 159) | (SEQ ID NO: 160) | (SEQ ID NO: 161) | (SEQ ID NO: 162) |
| 22: | WWWEEWWEE | WWWRRWWRR | AAAEEAAEE | AAARRAARR |
| | (SEQ ID NO: 163) | (SEQ ID NO: 164) | (SEQ ID NO: 165) | (SEQ ID NO: 166) |
| 23: | WWEEWWWEE | WWRRWWWRR | AAEEAAAEE | AARRAAARR |
| | (SEQ ID NO: 167) | (SEQ ID NO: 168) | (SEQ ID NO: 169) | (SEQ ID NO: 170) |
| 24: | WEEWWWWEE | WRRWWWWRR | AEEAAAAEE | ARRAAAARR |
| | (SEQ ID NO: 171) | (SEQ ID NO: 172) | (SEQ ID NO: 173) | (SEQ ID NO: 174) |
| 25: | WWWWEEEWE | WWWWRRRWR | AAAAEEEAE | AAAARRRAR |
| | (SEQ ID NO: 175) | (SEQ ID NO: 176) | (SEQ ID NO: 177) | (SEQ ID NO: 178) |
| 26: | WWWEEEWWE | WWWRRRWWR | AAAEEEAAE | AAARRRAAR |
| | (SEQ ID NO: 179) | (SEQ ID NO: 180) | (SEQ ID NO: 181) | (SEQ ID NO: 182) |
| 27: | WWEEEWWWE | WWRRRWWWR | AAEEEAAAE | AARRRAAAR |
| | (SEQ ID NO: 183) | (SEQ ID NO: 184) | (SEQ ID NO: 185) | (SEQ ID NO: 186) |
| 28: | WEEEWWWWE | WRRRWWWWR | AEEEAAAAE | ARRRAAAAR |
| | (SEQ ID NO: 187) | (SEQ ID NO: 188) | (SEQ ID NO: 189) | (SEQ ID NO: 190) |
| 29: | EWWWWEEWE | RWWWWRRWR | EAAAAEEAE | RAAAARRAR |
| | (SEQ ID NO: 191) | (SEQ ID NO: 192) | (SEQ ID NO: 193) | (SEQ ID NO: 194) |
| 30: | EWWWEEWWE | RWWWRRWWR | EAAAEEAAE | RAAARRAAR |
| | (SEQ ID NO: 195) | (SEQ ID NO: 196) | (SEQ ID NO: 197) | (SEQ ID NO: 198) |
| 31: | EWWEEWWWE | RWWRRWWWR | EAAEEAAAE | RAARRAAAR |
| | (SEQ ID NO: 199) | (SEQ ID NO: 200) | (SEQ ID NO: 201) | (SEQ ID NO: 202) |
| 32: | EWEEWWWWE | RWRRWWWWR | EAEEAAAAE | RARRAAAAR |
| | (SEQ ID NO: 203) | (SEQ ID NO: 204) | (SEQ ID NO: 205) | (SEQ ID NO: 206) |
| 33: | EEWWWWEEW | RRWWWWRRW | EEAAAAEEA | RRAAAARRA |
| | (SEQ ID NO: 207) | (SEQ ID NO: 208) | (SEQ ID NO: 209) | (SEQ ID NO: 210) |
| 34: | EEWWWEEWW | RRWWWRRWW | EEAAAEEAA | RRAAARRAA |
| | (SEQ ID NO: 211) | (SEQ ID NO: 212) | (SEQ ID NO: 213) | (SEQ ID NO: 214) |
| 35: | EEWWEEWWW | RRWWRRWWW | EEAAEEAAA | RRAARRAAA |
| | (SEQ ID NO: 215) | (SEQ ID NO: 216) | (SEQ ID NO: 217) | (SEQ ID NO: 218) |
| 36: | EEWEEWWWW | RRWRRWWWW | EEAEEAAAA | RRARRAAAA |
| | (SEQ ID NO: 219) | (SEQ ID NO: 220) | (SEQ ID NO: 221) | (SEQ ID NO: 222) |
| 37: | EWWWWEEEW | RWWWWRRRW | EAAAAEEEA | RAAAARRRA |
| | (SEQ ID NO: 223) | (SEQ ID NO: 224) | (SEQ ID NO: 225) | (SEQ ID NO: 226) |
| 38: | EWWWEEEWW | RWWWRRRWW | EAAAEEEAA | RAAARRRAA |
| | (SEQ ID NO: 227) | (SEQ ID NO: 228) | (SEQ ID NO: 229) | (SEQ ID NO: 230) |
| 39: | EWWEEEWWW | RWWRRRWWW | EAAEEEAAA | RAARRRAAA |
| | (SEQ ID NO: 231) | (SEQ ID NO: 232) | (SEQ ID NO: 233) | (SEQ ID NO: 234) |
| 40: | EWEEEWWWW | RWRRRWWWW | EAEEEAAAA | RARRRAAAA |
| | (SEQ ID NO: 235) | (SEQ ID NO: 236) | (SEQ ID NO: 237) | (SEQ ID NO: 238) |
| 41: | WWWEWEWEE | WWWRWRWRR | AAAEAEAEE | AAARARARR |
| | (SEQ ID NO: 239) | (SEQ ID NO: 240) | (SEQ ID NO: 241) | (SEQ ID NO: 242) |
| 42: | WWEWEWWEE | WWRWRWWRR | AAEAEAAEE | AARARAARR |
| | (SEQ ID NO: 243) | (SEQ ID NO: 244) | (SEQ ID NO: 245) | (SEQ ID NO: 246) |
| 43: | WEWEWWWEE | WRWRWWWRR | AEAEAAAEE | ARARAAARR |
| | (SEQ ID NO: 247) | (SEQ ID NO: 248) | (SEQ ID NO: 249) | (SEQ ID NO: 250) |
| 44: | EWEWWWWEE | RWRWWWWRR | EAEAAAAEE | RARAAAARR |
| | (SEQ ID NO: 251) | (SEQ ID NO: 252) | (SEQ ID NO: 253) | (SEQ ID NO: 254) |
| 45: | EWWWEWEWE | RWWWRWRWR | EAAAEAEAE | RAAARARAR |
| | (SEQ ID NO: 255) | (SEQ ID NO: 256) | (SEQ ID NO: 257) | (SEQ ID NO: 258) |
| 46: | EWWEWEWWE | RWWRWRWWR | EAAEAEAAE | RAARARAAR |
| | (SEQ ID NO: 259) | (SEQ ID NO: 260) | (SEQ ID NO: 261) | (SEQ ID NO: 262) |
| 47: | EWEWEWWWE | RWRWRWWWR | EAEAEAAAE | RARARAAAR |
| | (SEQ ID NO: 263) | (SEQ ID NO: 264) | (SEQ ID NO: 265) | (SEQ ID NO: 266) |
| 48: | EEWEWWWWE | RRWRWWWWR | EEAEAAAAE | RRARAAAAR |
| | (SEQ ID NO: 267) | (SEQ ID NO: 268) | (SEQ ID NO: 269) | (SEQ ID NO: 270) |
| 49: | EEWWWEWEW | RRWWWRWRW | EEAAAEAEA | RRAAARARA |
| | (SEQ ID NO: 271) | (SEQ ID NO: 272) | (SEQ ID NO: 273) | (SEQ ID NO: 274) |
| 50: | EEWWEWEWW | RRWWRWRWW | EEAAEAEAA | RRAARARAA |
| | (SEQ ID NO: 275) | (SEQ ID NO: 276) | (SEQ ID NO: 277) | (SEQ ID NO: 278) |
| 51: | EEWEWEWWW | RRWRWRWWW | EEAEAEAAA | RRARARAAA |
| | (SEQ ID NO: 279) | (SEQ ID NO: 280) | (SEQ ID NO: 281) | (SEQ ID NO: 282) |
| 52: | EEEWEWWWW | RRRWRWWWW | EEEAEAAAA | RRRARAAAA |
| | (SEQ ID NO: 283) | (SEQ ID NO: 284) | (SEQ ID NO: 285) | (SEQ ID NO: 286) |
| 53: | WEWWEWWEE | WRWWRWWRR | AEAAEAAEE | ARAARAARR |
| | (SEQ ID NO: 287) | (SEQ ID NO: 288) | (SEQ ID NO: 289) | (SEQ ID NO: 290) |
| 54: | EWWEWWWEE | RWWRWWWRR | EAAEAAAEE | RAARAAARR |
| | (SEQ ID NO: 291) | (SEQ ID NO: 292) | (SEQ ID NO: 293) | (SEQ ID NO: 294) |
| 55: | EWEWWEWWE | RWRWWRWWR | EAEAAEAAE | RARAARAAR |
| | (SEQ ID NO: 295) | (SEQ ID NO: 296) | (SEQ ID NO: 297) | (SEQ ID NO: 298) |
| 56: | EEWWEWWWE | RRWWRWWWR | EEAAEAAAE | RRAARAAAR |
| | (SEQ ID NO: 299) | (SEQ ID NO: 300) | (SEQ ID NO: 301) | (SEQ ID NO: 302) |
| 57: | WWEEWWEEW | WWRRWWRRW | AAEEAAEEA | AARRAARRA |
| | (SEQ ID NO: 303) | (SEQ ID NO: 304) | (SEQ ID NO: 305) | (SEQ ID NO: 306) |
| 58: | WEEWWEEWW | WRRWWRRWW | AEEAAEEAA | ARRAARRAA |
| | (SEQ ID NO: 307) | (SEQ ID NO: 308) | (SEQ ID NO: 309) | (SEQ ID NO: 310) |
| 59: | EEWWEEWWW | RRWWRRWWW | EEAAEEAAA | RRAARRAAA |
| | (SEQ ID NO: 311) | (SEQ ID NO: 312) | (SEQ ID NO: 313) | (SEQ ID NO: 314) |
| 60: | EWWEWWEEW | RWWRWWRRW | EAAEAAEEA | RAARAARRA |
| | (SEQ ID NO: 315) | (SEQ ID NO: 316) | (SEQ ID NO: 317) | (SEQ ID NO: 318) |
| 61: | WWEWWEWEW | WWRWWRWRW | AAEAAEAEA | AARAARARA |
| | (SEQ ID NO: 319) | (SEQ ID NO: 320) | (SEQ ID NO: 321) | (SEQ ID NO: 322) |
| 62: | EEWWEWWEW | RRWWRWWRW | EEAAEAAEA | RRAARAARA |
| | (SEQ ID NO: 323) | (SEQ ID NO: 324) | (SEQ ID NO: 325) | (SEQ ID NO: 326) |
| 63: | EWEWWEEWW | RWRWWRRWW | EAEAAEEAA | RARAARRAA |
| | (SEQ ID NO: 327) | (SEQ ID NO: 328) | (SEQ ID NO: 329) | (SEQ ID NO: 330) |
| 64: | EEWWEWEWW | RRWWRWRWW | EEAAEAEAA | RRAARARAA |
| | (SEQ ID NO: 331) | (SEQ ID NO: 332) | (SEQ ID NO: 333) | (SEQ ID NO: 334) |
| 65: | WWEWEWEWE | WWRWRWRWR | AAEAEAEAE | AARARARAR |
| | (SEQ ID NO: 335) | (SEQ ID NO: 336) | (SEQ ID NO: 337) | (SEQ ID NO: 338) |
| 66: | WEWEWEWEW | WRWRWRWRW | AEAEAEAEA | ARARARARA |
| | (SEQ ID NO: 339) | (SEQ ID NO: 340) | (SEQ ID NO: 341) | (SEQ ID NO: 342) |
| 67: | EWEWEWEWW | RWRWRWRWW | EAEAEAEAA | RARARARAA |
| | (SEQ ID NO: 343) | (SEQ ID NO: 344) | (SEQ ID NO: 345) | (SEQ ID NO: 346) |
| 68: | WEWEWWEEW | WRWRWWRRW | AEAEAAEEA | ARARAARRA |
| | (SEQ ID NO: 347) | (SEQ ID NO: 348) | (SEQ ID NO: 349) | (SEQ ID NO: 350) |
| 69: | WEWEWEEWW | WRWRWRRWW | AEAEAEEAA | ARARARRAA |
| | (SEQ ID NO: 351) | (SEQ ID NO: 352) | (SEQ ID NO: 353) | (SEQ ID NO: 354) |
| 70: | WEWEEEWWW | WRWRRRWWW | AEAEEEAAA | ARARRRAAA |
| | (SEQ ID NO: 355) | (SEQ ID NO: 356) | (SEQ ID NO: 357) | (SEQ ID NO: 358) |
| 71: | WEEEEWWWW | WRRRRWWWW | AEEEEAAAA | ARRRRAAAA |
| | (SEQ ID NO: 359) | (SEQ ID NO: 360) | (SEQ ID NO: 361) | (SEQ ID NO: 362) |
| 72: | WWEEEEWWW | WWRRRRWWW | AAEEEEAAA | AARRRRAAA |
| | (SEQ ID NO: 363) | (SEQ ID NO: 364) | (SEQ ID NO: 365) | (SEQ ID NO: 366) |
| 73: | WWWEEEEWW | WWWRRRRWW | AAAEEEEAA | AAARRRRAA |
| | (SEQ ID NO: 367) | (SEQ ID NO: 368) | (SEQ ID NO: 369) | (SEQ ID NO: 370) |
| 74: | WWWWEEEEW | WWWWRRRRW | AAAAEEEEA | AAAARRRRA |
| | (SEQ ID NO: 371) | (SEQ ID NO: 372) | (SEQ ID NO: 373) | (SEQ ID NO: 374) |
| 75: | WWWEWEEEW | WWWRWRRRW | AAAEAEEEA | AAARARRRA |
| | (SEQ ID NO: 375) | (SEQ ID NO: 376) | (SEQ ID NO: 377) | (SEQ ID NO: 378) |
| 76: | WEWEEEWWW | WRWRRRWWW | AEAEEEAAA | ARARRRAAA |
| | (SEQ ID NO: 379) | (SEQ ID NO: 380) | (SEQ ID NO: 381) | (SEQ ID NO: 382) |
| 77: | WWWEEWEEW | WWWRRWRRW | AAAEEAEEA | AAARRARRA |
| | (SEQ ID NO: 383) | (SEQ ID NO: 384) | (SEQ ID NO: 385) | (SEQ ID NO: 386) |
| 78: | WWEEWEEWW | WWRRWRRWW | AAEEAEEAA | AARRARRAA |
| | (SEQ ID NO: 387) | (SEQ ID NO: 388) | (SEQ ID NO: 389) | (SEQ ID NO: 390) |
| 79: | WEEWEEWWW | WRRWRRWWW | AEEAEEAAA | ARRARRAAA |
| | (SEQ ID NO: 391) | (SEQ ID NO: 392) | (SEQ ID NO: 393) | (SEQ ID NO: 394) |
| 80: | WWWEEEWEW | WWWRRRWRW | AAAEEEAEA | AAARRRARA |
| | (SEQ ID NO: 395) | (SEQ ID NO: 396) | (SEQ ID NO: 397) | (SEQ ID NO: 398) |
| 81: | WWEEEWEWW | WWRRRWRWW | AAEEEAEAA | AARRRARAA |
| | (SEQ ID NO: 399) | (SEQ ID NO: 400) | (SEQ ID NO: 401) | (SEQ ID NO: 402) |
| 82: | WEEEWEWWW | WRRRWRWWW | AEEEAEAAA | ARRRARAAA |
| | (SEQ ID NO: 403) | (SEQ ID NO: 404) | (SEQ ID NO: 405) | (SEQ ID NO: 406) |
| 83: | WEEEWWEWW | WRRRWWRWW | AEEEAAEAA | ARRRAARAA |
| | (SEQ ID NO: 407) | (SEQ ID NO: 408) | (SEQ ID NO: 409) | (SEQ ID NO: 410) |
| 84: | WEEEWWWEW | WRRRWWWRW | AEEEAAAEA | ARRRAAARA |
| | (SEQ ID NO: 411) | (SEQ ID NO: 412) | (SEQ ID NO: 413) | (SEQ ID NO: 414) |
| 85: | WEEWWEEWW | WRRWWRRWW | AEEAAEEAA | ARRAARRAA |
| | (SEQ ID NO: 415) | (SEQ ID NO: 416) | (SEQ ID NO: 417) | (SEQ ID NO: 418) |
| 86: | WEEWWWEEW | WRRWWWRRW | AEEAAAEEA | ARRAAARRA |
| | (SEQ ID NO: 419) | (SEQ ID NO: 420) | (SEQ ID NO: 421) | (SEQ ID NO: 422) |

One of ordinary skill in the art will readily recognize that parameters of peptides may be altered by altering the combination of amino acid residues in the sequence. For example, using the hydrophobicity values in Table 1, the sequence VWYAA (SEQ ID NO:423) has as summed hydrophobicity value of 5.05, the sequence MAMYA (SEQ ID NO:424) has a summed hydrophobicity of 4.04, and the sequence AFAAA has a summed hydrophobicity of 3.03, giving a step size in this parameter of 1.01. Likewise, one of ordinary skilled in the art will readily recognize that it is possible to adjust the charge of a peptide by altering its sequence. For example, the sequence KKSK (SEQ ID NO:425) has a formal charge of +3, the sequence KSQR (SEQ ID NO:426) has a formal charge of +2, the sequence RTSQ (SEQ ID NO:427) has a formal charge of +1, and the sequence NTSQ (SEQ ID NO:428) has a formal charge of 0. Incorporating measures of the atomic partial charges of the various sidechains, such as those represented within the AMBER forcefield (Cornell et al. (1995) J. Am. Chem. Soc. 117: 5179-5197) or other similar parameter sets, provides a means of subdividing charges further, allowing smaller stepsizes to be achieved.

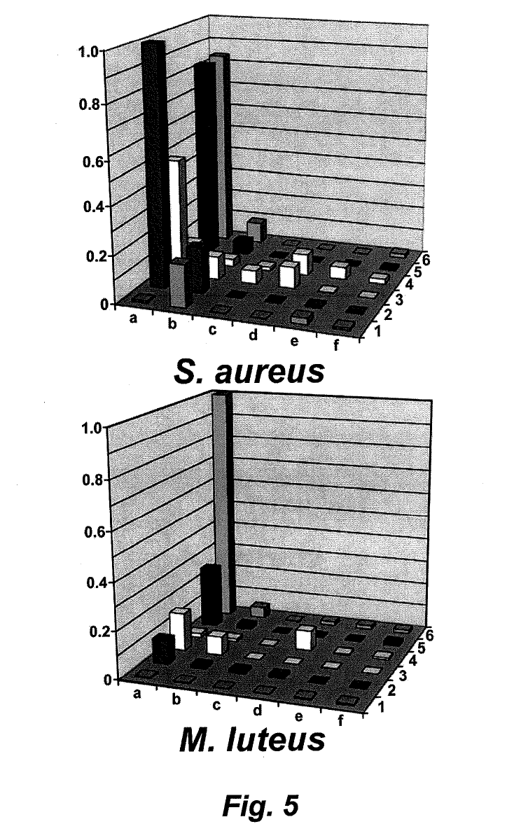

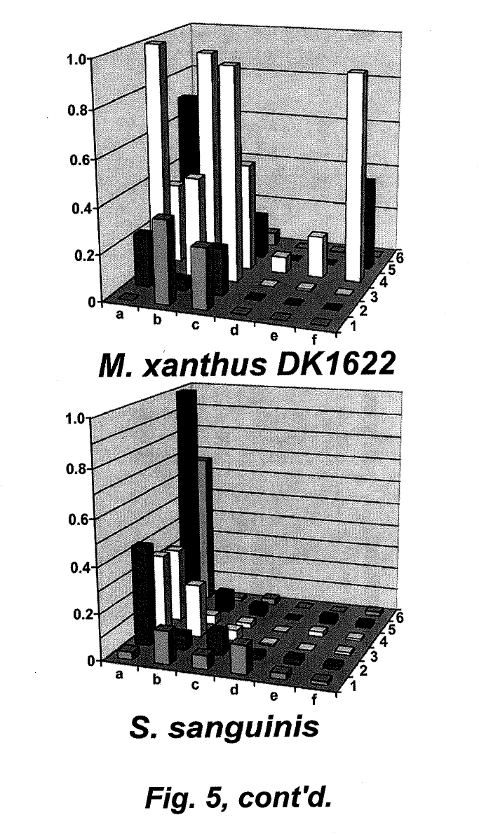

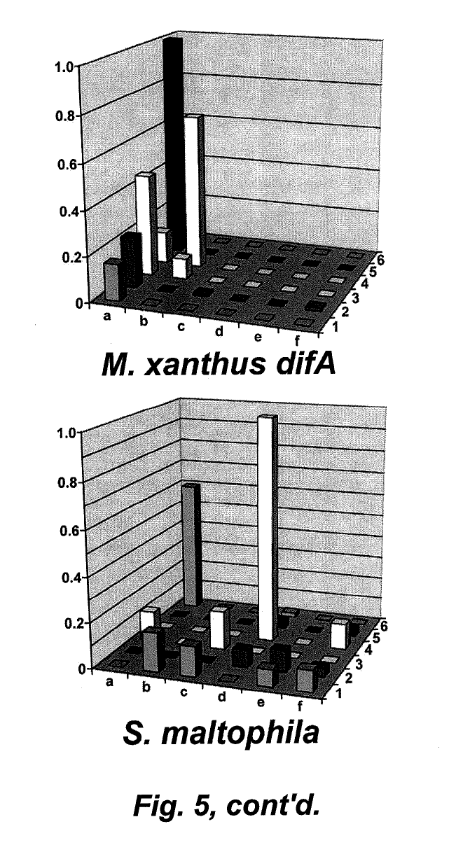

The “sparse matrix” libraries described herein are useful in a wide variety of contexts. For example, they can be used to select receptor agonists or antagonists, enzyme inhibitors, new antigens, affinity ligands, antagonists of protein-protein interactions and catalysts, and moieties that bind particular microorganisms, tissues and/or cells. Libraries can thus be seen as a source of bioactive molecules that are selected on the basis of the biochemical pre-defined by appropriate library parameters. In general, the more diverse the library, the higher the probability of selecting select good “hits”. Accordingly, in certain embodiments, the sparse matrix library is designed to provide “high diversity”, sometimes at the expense of high complexity” (number of different components). Thus, in certain embodiments, it is desirable to maximize diversity while reducing the number of different building blocks that make up the matrix/library numbers. One possible simplification of peptide libraries attainable with natural amino acids in the L- or D configuration is described in Marasco et al. (2008) Current Protein and Peptide Science, 9: 447-467 (see, e.g., FIGS. 5A and 5B therein). As illustrated therein a set of “non redundant” amino acids is chosen following several simple rules. First of all “quasi-identical” groups of amino acids are selected: Asp and Glu; Asn and Gln; Ile and Val; Met and Leu; Arg and Lys; Gly and Ala; Tyr and Trp; Ser and Thr. Then, from each group, one is selected trying to keep a desired distribution of properties, including hydropathic properties, charge, pKa, aromaticity and the like. In certain embodiments cysteine is converted to the stable acetamidomethyl-derivative to prevent polymerization and to increase the number of polar residues within the final set.

The foregoing are simply illustrative considerations for the design of certain peptide (and other) libraries. Using the teachings provided herein, one of skill will readily be able to design peptide libraries, peptoid libraries, oligosaccharide/polysaccharide libraries and the like that vary systematically in one or more properties as described herein.

While the terms “longitudinal”; “latitudinal”, “corner”, “row”, “column” and the like are used with reference to various sparse matrix libraries, it will be appreciated that these terms are used for descriptive convenience and do not necessarily require the disposition of matrix members in a particular spatial configuration. In certain embodiments, the libraries can be provided as collections of individual library members, each in a separate container, as collections of library members in various containers, and/or, as members attached to one or more substrates.

The compounds comprising the libraries can be synthesized according to standard methods well known to those of skill in the art. Thus, for example, solid phase peptide synthesis in which, for example, the C-terminal amino acid of the sequence is attached to an insoluble support followed by sequential addition of the remaining amino acids in the sequence is described by Barany and Merrifield, Solid-Phase Peptide Synthesis; pp. 3-284 in The Peptides Analysis, Synthesis, Biology. Vol. 2: Special Methods in Peptide Synthesis, Part A., Merrifield, et al. (1963) J. Am. Chem. Soc., 85: 2149-2156, and Stewart et al. (1984) Solid Phase Peptide Synthesis, 2nd ed. Pierce Chem. Co., Rockford, Ill., and the like. Similarly, methods for the simultaneous synthesis of complex libraries of peptides are also known to those of skill (see, e.g., Ede, (2001) J. Immunol. Meth., 267(1): 3-11, and references cited therein).

In the SPOT synthesis (see, e.g., Kramer et al. (1993) J. Pept. Res., 6: 314-319; Frank (1994) in Innovations and Perspectives in Solid Phase Synthesis, 3: 509 Mayflower Worldwide Ltd. Birmingham), peptides arrays are synthesized in a stepwise manner on a flat solid support, such as functionalized cellulose membrane, polypropylene, ceramic, quartz, glass, etc., following the standard Fmoc-based peptide chemistry (Frank (1992) Tetrahedron, 48: 9217; Min et al. (2004) Angew. Chem. Int. Ed., 43: 5973-5980). The support is selected to meet the chemical and the biological requirements of the target and for compatibility with the synthesis and screening methods. It also influences the functionalization type and the insertion of spacers and/or linkers (Frank and Overwin (1996) Methods Mol. Biol., 66: 149-169; Kramer et al. (1999) J. Pept. Res., 54: 319-327; Wenschuh et al. (2000) Biopolymers, 55: 188-206). Illustrative supports include, but are not limited to ester-derivatized planar supports, CAPE [celluloseamino-hydroxypropyl ether] membranes, amino-functionalized polypropylene membranes (Volkmer-Engert et al. (1997) Tetrahedron Lett., 38: 1029-1032; Licha et al. (2000) Tetrahedron Lett., 41: 1711-1715), glass surfaces (Falsey et al. (2001) Bioconjug. Chem., 12, 346-353; Lesaicherre et al. (2002) Bioorg. Med. Chem. Lett., 12: 2085-2088; Takahashi et al. (2003) Chem. Biol., 10: 53-60), and the like. Where peptides are synthesized on spatially confined spots, the x-y position on a planar support also represents the library code which is exploited to deconvolute the libraries.

Methods for the synthesis of modified peptides (e.g., peptoids) are also well known to those of skill in the art. Thus, for example, replacement of the peptide (amide bonds) with a saturated amine is described in U.S. Pat. No. 4,496,542, which is incorporated herein by reference for all purposes, and by Kaltenbronn et al. (1990) Pp. 969-970 Proc. 11th American Peptide Symp., ESCOM Science Publishers, The Netherlands. Suh et al. (1985) Eur J Med Chem—Chim Ther 20: 563-570, disclosed peptides in which the Gly amide nitrogen was substituted with 2,3-dihydro-1H-indene. Sempuku et al. (1984) Chem. Abs. 100: 68019b, disclosed N-substituted glycine derivatives. U.S. Pat. No. 6,0785,121 and PCT Publication WO 91/19735, both of which are incorporated herein by reference disclose the synthesis of peptoids comprising N-substituted glycines, while U.S. Pat. No. 4,897,445 describes the synthesis of peptides containing non-peptide bonds (e.g., —CO—NH— bonds). A variety of enzymes (proteases) can act on this bond and hydrolyze it to break the polypeptide chain into two or more fragments. U.S. Pat. No. 5,646,285 discloses the preparation of peptide analogs containing thioamide, vinylogous amide, hydrazino, methyleneoxy, thiomethylene, phosphonamides, oxyamide, hydroxyethylene, reduced amide and substituted reduced amide isosteres and β-sulfonamide(s) (see, also, Spatola (1983) Chem. Biochem. Amino Acids and Proteins 7: 267-357). These methods are illustrative and not limiting. Methods for the synthesis of numerous other modified peptides are well known to those of skill in the art.

Methods for the stepwise synthesis of oligosaccharides are also known. For example, U.S. Pat. No. 7,160,517 describes apparatus and methods for the automated synthesis of oligosaccharides. Other protocols can be found for example in Seeberger et al. (1999) Organic Letts., 1(11): 1811-1814; Plante et al. (1999) Organic Letts 1(2): 211-214; Osborn and Khan (1999) Tetrahedron 55: 1807-1850; St Hilaire and Meldal (2000) Angew. Chem. Int. Ed. 39: 1162-1179; Wang and Hindsgaul (1999) Glycoimmunology 2: 219-236; (1999); and the like.

The members of the sparse matrix library can be provided in separate containers (e.g., one container per species) or pooled or subpooled into various containers, or disposed in wells, e.g., on a microtiter or other plate, or disposed on, adsorbed to, or covalently coupled to a solid substrate. When disposed on or attached to a substrate, the substrate can be contiguous, e.g., one substrate surface having attached thereto all of the members of the library, or discontinuous, each substrate providing a sublibrary or even an individual member of a library. In certain instances, the substrate comprises a bead or nanoparticle.

Many strategies can be utilized to encode peptide and non-peptide libraries. Chemical encodings performed during the synthesis steps inserting small amounts of “readable” tags, such as small alogenated aromatics or short DNA stretches, have been described (see, e.g., Ohlmeyer et al. (1993) Proc. Natl. Acad. Sci., USA, 90: 10922-10923; Brenner and Lerner (1992) Proc. Natl. Acad. Sci., USA, 89(12): 5381-5383). In addition, short peptide sequences have been employed to encode non-peptidic libraries (see, e.g., Kerr et al. (1993) J. Am. Chem. Soc., 115: 2529-2531). Physical labeling using radio chips (Geysen et al. (2003) Nature Reviews Drug Discovery 2: 222-230) was also introduced as a valuable and elegant method to track the chemical history of library components (Xiao et al. (2000) Biotechnol. Bioeng., 71(1): 44-50; Cheung et al. (2005) Bioorg. Med. Chem. Lett., 15(24): 5504-5508; Sun et al. (2007) Bioorg. Med. Chem. Lett., 17(24): 6899) and in some cases also to perform the on-bead selection. Physical and chemical labeling has evolved into SYNPHASE® Lanterns (Zajdel et al. (2006) QSAR Comb. Sci., 26(2): 215-219), ECLiPS® technology (Dolle et al. (2000) J. Comb. Chem., 2: 716-731), and the like. ECLIPS® methods use readable tags that record the reaction sequence. Each different combination of tags encodes a different molecule on the synthesis support. Therefore once removed from the common solid support, analysis of the tags provides information on the building block types and their location within the molecular structure (Kerr et al. (1993) J. Am. Chem. Soc., 115: 2529-2531).

When disposed on a contiguous substrate, in certain embodiments, the library members are spatially addressed so that each member of the library can be identified by its location (e.g., coordinates) on the substrate.

In certain embodiments, the library members can be joined to a surface utilizing a biotin/avidin interaction. In certain embodiments biotin or avidin, e.g. with a photolabile protecting group can be affixed to the surface library members. Irradiation of the surface in the presence of the desired library member bearing the corresponding avidin or streptavidin, or biotin, results in coupling of the member to the surface.

Where the surface and/or the library member bear reactive groups or are derivatized to bear reactive groups numerous coupling methods are readily available. Thus, for example, a free amino group is amenable to acylation reactions with a wide variety of carboxyl activated linker extensions that are well known to those skilled in the art. Linker extension can performed at this stage to generate terminal activated groups such as active esters, isocyanates, maleimides, and the like. For example, reaction of the peptide or amino-derivatized surface with one end of homobifunctional N-hydroxysuccinimide esters of bis-carboxylic acids such as terephthalic acid will generate stable N-hydroxysuccinimide ester terminated linker adducts that useful for conjugation to amines. Linker extension can also be accomplished with heterobifunctional reagents such as maleimido alkanoic acid N-hydroxysuccinimide esters to generate terminal maleimido groups for subsequent conjugation to thiol groups. An amino-terminated linker can be extended with a heterobifunctional thiolating reagent that reacts to form an amide bond at one end and a free or protected thiol at the other end. Some examples of thiolating reagents of this type which are well known in the art are 2-iminothiolane (2-IT), succinimidyl acetylthiopropionate (SATP) and succinimido 2-pyridyldithiopropionate (SPDP). The incipient thiol group is then available, after deprotection, to form thiol ethers with maleimido or bromoacetylated moieties or to interact directly with a gold surface. In various embodiments the amino group, e.g., of an amino-terminated linker can be converted a diazonium group and hence the substance into a diazonium salt, for example, by reaction with an alkali metal nitrite in the presence of acid, which is then reactive with a suitable nucleophilic moiety, such as, but not limited to, the tyrosine residues of peptides, and the like. Examples of suitable amino-terminated linkers for conversion to such diazonium salts include, but are not limited to aromatic amines (anilines), and may also include the aminocaproates and similar substances referred to above. Such anilines can readily be obtained by substituting into the coupling reaction between the an available hydroxyl group and an N-protected amino acid, as discussed above, the corresponding amino acid wherein the amino group is comprised of an aromatic amine, that is, an aniline, with the amine suitably protected, for example, as an N-acetyl or N-trifluoroacetyl group, which is then deprotected using methods well-known in the art. Other suitable amine precursors to diazonium salts will be suggested to one skilled in the art of organic synthesis.

Another favored type of heterobifunctional linker is a mixed active ester/acid chloride such as succinimido-oxycarbonyl-butyryl chloride. The more reactive acid chloride end of the linker preferentially acylates amino or hydroxyl groups, e.g., on the peptide to give N-hydroxysuccinimidyl ester linker adducts directly.

Yet another type of terminal activated group useful in the present invention is an aldehyde group. Aldehyde groups may be generated by coupling a free hydroxyl (e.g. on a peptide or derivatized nanocrescent) with an alkyl or aryl acid substituted at the omega position (the distal end) with a masked aldehyde group such as an acetal group, such as 1,3-dioxolan-2-yl or 1,3-dioxan-2-yl moieties, followed by unmasking of the group using methods well-known in the art. In various embodiments alkyl or aryl carboxylic acids substituted at the omega position with a protected hydroxy, such as, for example, an acetoxy moiety, may be used in coupling reactions, followed by deprotection of the hydroxy and mild oxidation with a reagent such as pyridinium dichromate in a suitable solvent, preferably methylene chloride, to give the corresponding aldehyde. Other methods of generating aldehyde-terminated substances will be apparent to those skilled in the art.

In certain embodiments, the libraries are attached to a surface to provide a high density array of library members. In certain embodiments such an array can comprise at least 100 or at least 200 different members/cm², preferably at least 300, 400, 500, or 1000 different members/cm², and more preferably at least 1,500, 2,000, 4,000, or 10,000 different members/cm².

Methods of patterning molecules on surfaces at high density are well known to those of skill in the art. Such methods include, for example, the use of high density microarray printers which are essentially spotting printers (see, e.g., Heller (2002) Annu Rev Biomed Eng. 4: 129-153). Other microarray printers utilize “on-demand” piezoelectric droplet generators (e.g., inkjet printers) (see, e.g., U.S. Pat. Nos. 6,395,562; 6,365,378; 6,228,659 and 5,338, 688 and WO publications WO 95/251116 and WO/2003/028868 which are incorporated herein by reference. Other approaches involve de novo synthesis in place (see, e.g., Fodor et al. (1991) Science, 251: 767-773, and U.S. Pat. Nos. 6,269,846, 6,271,957, and 6,480,324 which are incorporated herein by reference). A number of array printers are commercially available (see, e.g., VERSA Mini Spot-printing Workstation from Aurora Biomed, BIOODYSSEY® CALLIGRAPHER® MINIARRAYER from Bio-Rad, QARRAY MINI® from Genetix, BIOROBOTICS MICROGRID® from Genomic Solutions, OMNIGRID ACCENT® from Genomic Solutions, and the like).

In certain embodiments the members of the sparse matrix library are labeled with a detectable label. Detectable labels suitable for use in the present invention include any composition detectable by spectroscopic, photochemical, biochemical, immunochemical, electrical, optical or chemical means. Useful labels in the present invention include, but are not limited to biotin for staining with labeled streptavidin conjugate, magnetic beads (e.g., Dynabeads™), fluorescent dyes (e.g., fluorescein, texas red, rhodamine, green fluorescent protein, quantum dots, and the like, see, e.g., Molecular Probes, Eugene, Oreg., USA), radiolabels (e.g., ³H, ¹²⁵I, ³⁵s, ¹⁴C, ³²P, etc.), enzymes (e.g., horse radish peroxidase, alkaline phosphatase and others commonly used in an ELISA), and calorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

### III. Uses of Sparse Matrix Libraries

The sparse matrix libraries described herein are useful in a number of different contexts. The libraries are particularly well suited to scanning a property/parameter space to identify library members that display one or more desired binding activities and/or biological and/or physical/chemical properties. Illustrative properties include, but are not limited to binding specificity and/or avidity, bactericidal activity, fungicidal activity, algacidal activity, virucidal activity, activity against protozoa, general microbicide activity, cytotoxicity, immunogenicity, binding specificity for species or strains of bacteria, archaea, fungi, alga, protozoa, biomaterials, biofilms biological surfaces, particular prokaryotic or eukaryotic cells, polymer surfaces_and the like.

Accordingly, in certain embodiments, on aspect of the present invention relates to a method of identifying a compound (e.g., a peptide or peptides) that specifically binds to a target, wherein the method comprises the steps of exposing or contacting a target with the members of a peptide matrix according to the present invention, measuring the interaction between the target with a peptide in the matrix, where the magnitude of the interaction in the presence of the target relative to the absence of the target indicates that the peptide specifically or preferentially binds to the target.

In certain embodiments library members (e.g., peptides) can be labeled with an appropriate label (e.g., fluorophore, dye, radioactive marker or spin-label, etc.) and exposed to the target, after which unbound peptide is washed away, and the amount of label bound to the target is assayed as appropriate (e.g., fluorescence measurements are made in the case of peptides conjugated to fluorescent labels). Alternatively, the library members can be immobilized on a solid support and exposed to a labeled or otherwise distinct target, followed by washing of the support and determination of the amount of target bound to the library-coated support(s). Many other screening methods are possible and the choice of screening method will depend on the specific target under consideration.

In another aspect of the present invention, the screening method further comprises a step of fine-tuning the matrix library through the use of the peptides identified that interact with the target(s). For example, in certain embodiments peptides that are identified as having shown the desired binding affinity for the target (hereafter called “hits”) can selected and subjected to further refinement as follows:

1. A new test library can be designed in which positions of the amino acids in the hits are changed yielding a small set of peptides with the same bulk properties but different sequences and conformations. For example, the periodicity with which the hydrophobic and hydrophilic peptides are placed is varied in systemic fashion as detailed in the examples.

2. Another new test library can be designed along the same lines of the pilot library, but in which the properties associated with each position are varied more subtly to provide a more refined probing of the region of the parameter space surrounding the hits (for example, given a pilot library in which the parameter of Charge is varied from −3 to +3 in steps of 1.0, and a hit with a charge of +2, a refined library might be generated in which this parameter is varied from +1.5 to +2.5 in steps of 0.2). The refined libraries are prepared, e.g., as described herein and screened for binding to the target as before. The process is continued until a suitable binding member (e.g., peptide) is identified.

Another aspect of the present invention relates to a method of identifying a pattern of interaction between a target and a sparse matrix library (e.g., a peptide library) of the present invention. In certain embodiments the method comprises contacting the library with a target and recording the interaction of the target with each library member in the matrix, where the collective interaction of the target with the matrix forms a pattern of interaction (or a fingerprint of the target with the matrix). The pattern can be used to determine the properties of the target in a given matrix or predict the nature of an unknown target when the pattern of the unknown target is substantially identical to the pattern of a known target.

Another aspect of the present invention relates to a method of identifying a target to be tested (or an unknown target) or identifying the property of the unknown target, wherein the method comprising the steps of contacting the target to be tested with a sparse matrix library of the present invention, determining a test pattern of interaction between the target to be tested and the matrix, and comparing the test pattern with a known pattern, wherein the known pattern is a fingerprint of a known target in the matrix and if the test pattern substantially matches the known pattern the target to be test is predicted to be the known target.

Another aspect of the present invention relates to a method of determining a peptide in the peptide library of the present invention which competes the binding of a compound to a target or binds competitively to the same epitope of the target as the compound. The method comprises the steps of contacting the a sparse matrix library (e.g., peptide library) with a target and a compound and measuring the level of interaction between a matrix compound and the target and between the compound and the target, wherein the increasing interaction of the peptide and the target and decreasing interaction of the target and the compound indicates that the peptide in the library competes the binding of the compound to the target. Additionally, the compound so identified can be an agonist or antagonist of the target by binding to the target through the same, similar or overlapping epitope as the compound.

### IV. Kits

In still another embodiment, this invention provides kits for practice of the assays or use of the sparse matrix libraries described herein. In certain embodiments, the kits comprise one or more containers containing the members of one or more sparse matrix libraries according to the present invention. In various embodiments the library members are disposed on or attached to a solid substrate, and/or disposed in one or more wells (e.g., of a microtiter plate) and/or on the surface of a culture dish, tube, flask, or the like. In certain embodiments the library members are optionally labeled with a detectable label.

Where the library members are provide in wells on a microtiter plate or disposed (spatially addressed) on a substrate, the kit can further optionally comprise a key providing the identity of each library member in the plate, surface, or well. The kits can optionally include any reagents and/or apparatus to facilitate practice of the assays described herein. Such reagents include, but are not limited to buffers, labels, labeled antibodies, labeled nucleic acids, filter sets for visualization of fluorescent labels, blotting membranes, and the like.

In addition, the kits can optionally include instructional materials containing directions (i.e., protocols) for the practice of assay/screening methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Designing a Peptide Matrix Library for Target Binding

The target specificity is derived from variation in surface charge and hydrophobicity across various biological surfaces, and the probability of obtaining a “hit” (which is the peptide that specifically interacts with or binds to the biological surface) is governed by the large number of binding modes possible on a surface that has the appropriate bulk physicochemical properties. It is contemplated, that by sampling the parameter space at extremely sparse intervals in the parameters of charge and hydrophobicity, a very small library (e.g., a library of 100 peptides) can be used to identify peptides that are bound to the target or the surfaces of the target. These initial “hits” provide information about the charge and hydrophobicity at the surface of interest, forming the basis of developing small refined libraries that provide fine-tuned levels of binding activity and specificity.

Two major aspects of the peptide isolation problem are considered with regard to complex surfaces: first, species or cell-type specificity in the bulk physicochemical properties of the cell surface (Dickson and Koohmaraie (1989) Appl Environ Microbiol 55(4): 832-836; Zita and Hermansson (1997) FEMS Microbiol Lett 152(2): 299-306; Nikaido (1999) J Bacteriol 181(1): 4-8) provides alternate sites for binding to cell surfaces that are not limited to the set of known cell-surface proteins but still confer species or cell-type specificity on the binding interaction; and second, because there are so many potential binding sites and alternate binding modes on the cell surface, there is a much higher likelihood that any given peptide will interact with the surface in some way. Importantly, this second point predicts that significant levels of surface binding can be obtained from very small libraries.

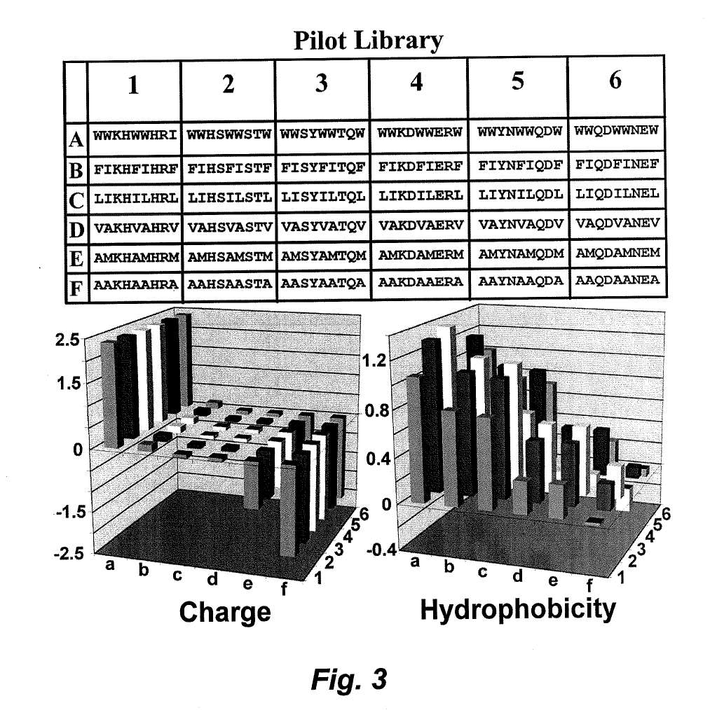

A pilot library in this example was designed consisting of 36 9-mer peptides. The parameters to be varied were hydrophobicity and charge, and the periodicity chosen was that of an amphipathic alpha helix, i.e. two hydrophobic residues followed by one charged residue. A 6×6 matrix was constructed: in one dimension, the amino acids at positions 1, 2, 5, 6, and 9 were varied from highly hydrophobic (predominantly Tryptophan, Phenylalanine, Leucine, and Isoleucine) to less hydrophobic (predominantly Alanine, Valine, and Methionine) (Fauchere and Pliska (1983) Eur. J. Med. Chem., 18: 369-375; Thorgeirsson et al. (1996) Biochemistry 35(6): 1803-1809), while in the second dimension the amino acids occupying positions 3, 4, 7, and 8 were varied from positively charged (predominantly Lysine, Arginine, and Histidine) to negatively charged (predominantly Aspartate and Glutamate). This resulted in a collection of 36 different peptide sequences with physical properties ranging from the combination of high hydrophobicity and positive charge (See FIG. 3: peptide A1—Grand Average of Hydropathicity (GRAVY)=1.056 (Fauchere and Pliska (1983) Eur. J. Med. Chem., 18: 369-375; Kyte and Doolittle (1982) J Mol Biol 157(1): 105-132), Charge=+4 (at pH 7.0)) to combined Low hydrophobicity and negative charge (peptide E6−GRAVY=−0.02, charge=−2 (at pH 7.0)).

### Example 2

### The Use of Peptide Matrix Library to Identify Peptides that Bind to Microbial Organisms or Generate a Finger Print for the Microbial Organisms

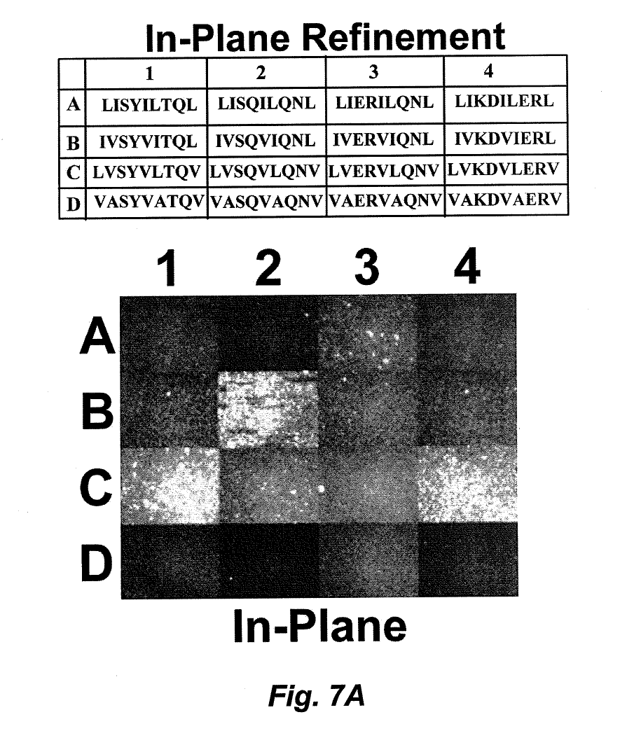

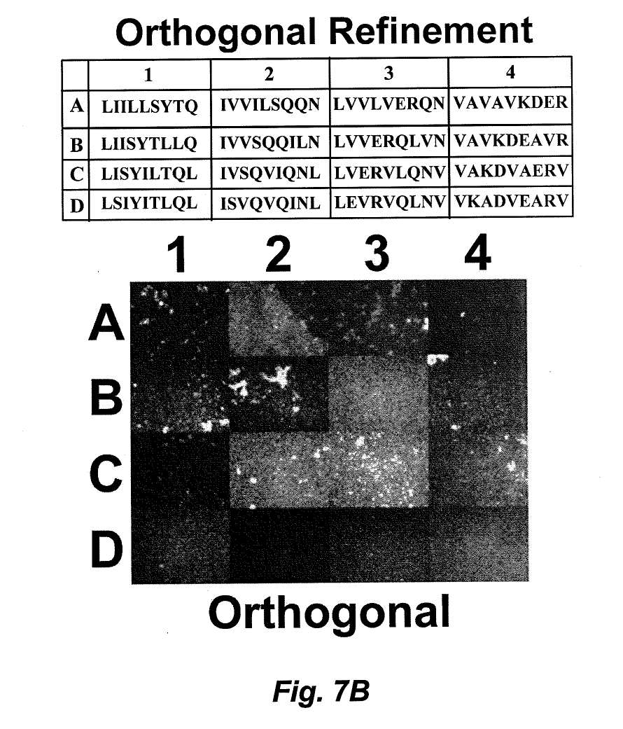

Peptides (sequences given in FIGS. 3 and 7) were synthesized using standard Fmoc solid phase chemistry on an Apex 396 multiple peptide synthesizer (AAPPTec, Louisville, Ky.) at 0.015 mM scale and labeled with 5(6)-carboxyfluorescein. Completed peptides were cleaved from the resin with 95% trifluoroacetic acid and appropriate scavengers.

Peptide samples were prepared at a concentration of 25 μM for screening against a variety of organisms by exposing immobilized bacteria to labeled peptides. For bacterial cell binding assays, cells were grown overnight, washed, and immobilized in a polylysine-coated 96-well plate. Fluorescein-labeled peptide samples were applied to immobilized bacteria, incubated for 10 minutes and washed extensively to remove unbound peptide. Samples were visualized by fluorescence microscopy. For each peptide, both brightfield and fluorescence images were collected, and camera and microscope settings were not altered between images. The locations of cells and background regions were determined using the brightfield images; those regions were then selected in the fluorescence images for quantitation of pixel values for determination of relative fluorescence intensities (represented as bacterial fluorescence/background fluorescence) using the GNU Image Manipulation Program (The GIMP, see, e.g., www.gimp.org).

The following bacterial species and strains were used: Lactobacillus casei (strain), Escherichia coli (W3110), Enterococcus faecium (strain), Streptococcus mutans (UA140), Streptococcus mitis (ATCC 903), Streptococcus gordonii (NY101), Pseudomonas aeruginosa(strain K), Klebsiella pneumoniae (KAY2026), Myxococcus xanthus (Strains DK1622, wildtype, and difA, exopolysaccharide deficient²¹), Micrococcus luteus (strain MS2, a community isolate), Staphylococcus epidermidis (strain), and Staphylococcus aureus(strains, and AM1, a methicillin/oxacillin-resistant community isolate).

For isolation of community-acquired S. aureus and M. luteus strains (strains MS2 and AM1), swabs were collected from the nostrils of several volunteers and streaked on Brain-Heart Infusion (BHI) agar and incubated at 37° C. overnight. Yellow colonies were isolated and typed (Microbial ID, Inc., Newark, Del.). Antibiotic resistance was assayed by plating on BHI in the presence of various antibiotics. Commercially available antibiotic susceptibility test disks were used to assay for oxacillin resistance (BD Biosciences), according to the manufacturer's instructions. Due to the difficulty of obtaining methicillin, oxacillin resistance was used as a proxy for methicillin resistance in isolated strains.²²

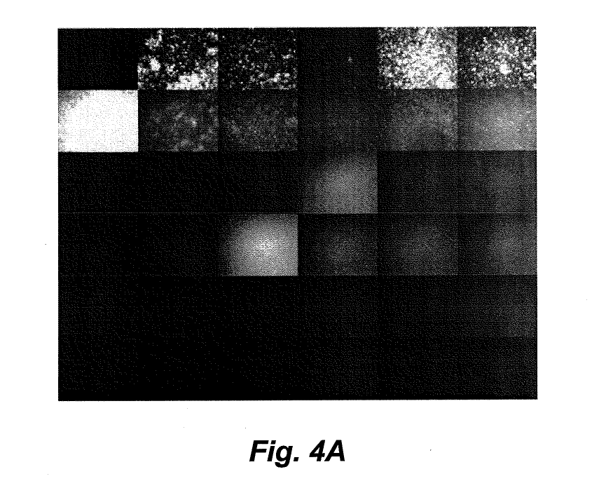

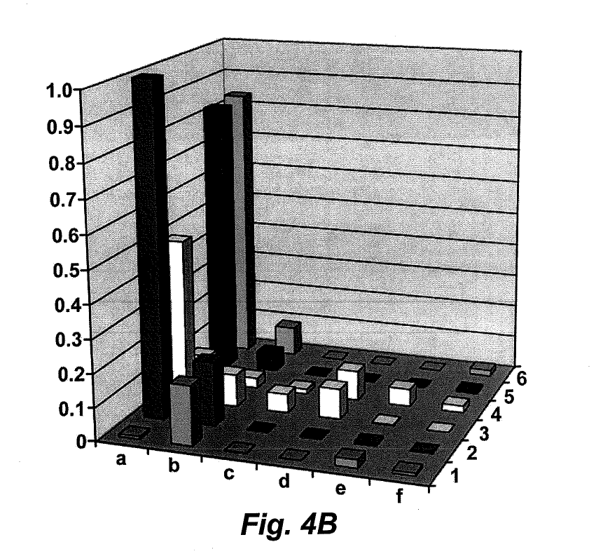

FIGS. 4A and 4B, for example, shows the binding of the pilot library to a community isolate of methicillin-resistant S. aureus, where each individual peptide shows a different level of binding to the target organism. Due to species- and strain-specific differences in the composition of the cell surface, different bacteria show different patterns of binding to the peptides presented in the pilot library, similar to the “fingerprints” observed with the binding of individual proteins to very large immobilized peptoid arrays (Reddy and Kodadek (2005) Proc. Natl. Acad. Sci., USA, 102 (36): 12672-12677). FIG. 5 shows the differences in the binding profiles between Staphylococcus aureus, Micrococcus luteus, Stenotrophomonas maltophila, Streptococcus sanguis, and both wild-type and Exopolysaccharide-deficient mutants of Myxococcus xanthus. M. luteus and S. aureus show similar but clearly distinguishable binding profiles, while the M. xanthus example is especially instructive, as the difference in binding profiles clearly reflects the difference between the wild-type M. xanthus cells and the DifA mutant strain which fails to produce exopolysaccharide, thus presenting a very different surface for peptide binding and providing a clear basis for the difference in binding profiles between these two isogenic strains. This offers a possible means of rapidly identifying bacterial species and strains.

### Example 3

### The Use of the Peptide Matrix Library on Non-Bacterial Surfaces

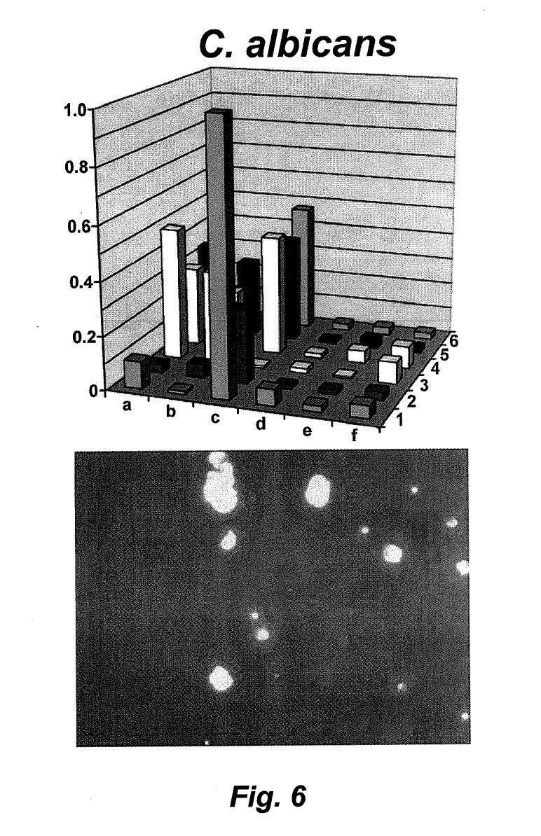

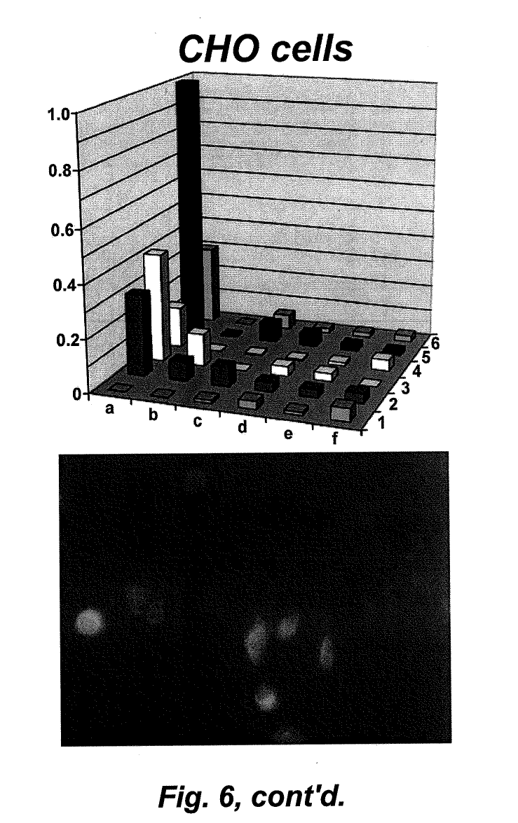

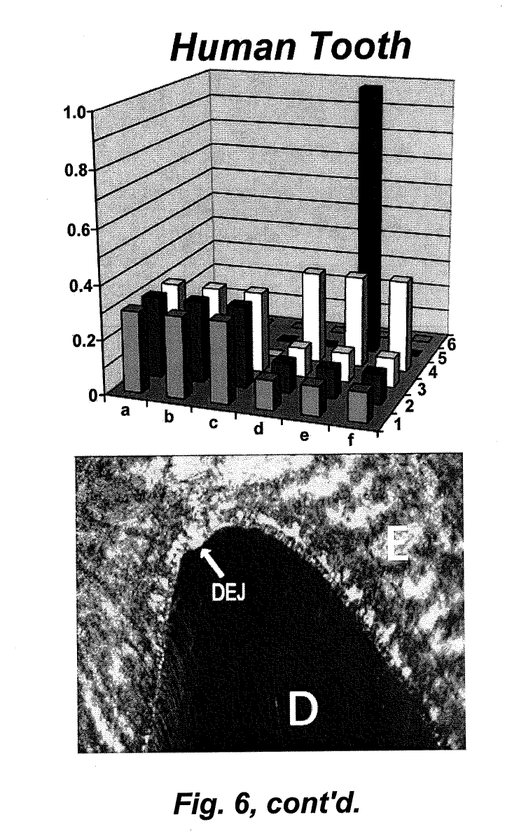

The use of this technology is not limited to bacterial surfaces. Similar results were seen in testing of the pilot library against Chinese Hamster Ovary (CHO) cells and Candida albicans cells (strain 4741, a clinical isolate), showing that this technique extends at least as far as fungal and mammalian cell surfaces (FIG. 6).

To further demonstrate the general applicability of this method, we screened the initial pilot library against the bioinorganic surface of a sectioned human tooth. The fluorescein-labeled peptides in the initial pilot library were pooled and screened by Confocal Laser Scanning Microscopy as follows. The quadrants of the library were collected into four pools of 9 peptides each. Peptides were screened by exposing the pooled, 5(6)-carboxyfluorescein-labeled peptides to sections taken from human teeth (extracted during normal clinical practice), followed by visualization of the samples by Confocal Laser Scanning Microscopy (CLSM). Pools that showed tissue-specific binding were divided into sub-pools of three peptides each, and the peptides comprising the sub-pools that showed the desired binding pattern were screened individually.

One quadrant showed specific staining of the enamel of sectioned human teeth, and splitting of the pools followed by further screening identified peptide 4F as the peptide responsible for this tissue-specific binding activity. As shown in FIG. 6, this peptide showed clear binding to the tooth enamel and to the enamel-proximal region of the dentino-enamel junction with no obvious binding to the dentin. The sequence of this peptide (AMKDAMERM, SEQ ID NO:429) does not contain any motifs that resemble known hydroxyapatite-binding sequences, but provides a specific interaction with the enamel, but not the chemically similar dentin.

This feature can be exploited in the clinical context. In particular, peptides comprising this sequence can be used to direct labels and/or therapeutic agents to hydroxyapatite surfaces in an organism. This can facilitate imaging of the surface and the preferential and/or specific delivery of a therapeutic agent thereto. This is particularly useful, for example, in guided restorative procedures where computers analyze the cavity preparation for completeness.

Accordingly, in certain embodiments, chimeric moieties are provided comprising a peptide comprising this sequence (or conservative substitutions thereof) attached directly or through a linker to a detectable label and/or to a therapeutic moiety or a composition comprising atherapeutic moiety.

Detectable labels suitable for use in such chimeric moieties include any composition detectable by spectroscopic, photochemical, biochemical, immunochemical, electrical, optical, or chemical means. Illustrative useful labels include, but are not limited to, biotin for staining with labeled streptavidin conjugates, avidin or streptavidin for labeling with biotin conjugates fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, ³²P, ⁹⁹Tc, ²⁰³Pb, ⁶⁷Ga, ⁶⁸Ga, ⁷²As, ¹¹¹In, ^{113m}In, ⁹⁷Ru, ⁶²Cu, 641Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, and the like), enzymes (e.g., horse radish peroxidase, alkaline phosphatase and others commonly used in an ELISA), various colorimetric labels, magnetic or paramagnetic labels (e.g., magnetic and/or paramagnetic nanoparticles), spin labels, radio-opaque labels, and the like. Patents teaching the use of such labels include, for example, U.S. Pat. Nos. 3,817,837; 3,850,752; 3,939,350; 3,996,345; 4,277,437; 4,275,149; and 4,366,241.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

In various embodiments spin labels are provided by reporter molecules with an unpaired electron spin which can be detected by electron spin resonance (ESR) spectroscopy. Illustrative spin labels include organic free radicals, transitional metal complexes, particularly vanadium, copper, iron, and manganese, and the like. Exemplary spin labels include, for example, nitroxide free radicals.

In certain embodiments the label can comprise one or more ligands, epitope tags, and/or antibodies. The terms “epitope tag” or “affinity tag” are used interchangeably herein, and used refers to a molecule or domain of a molecule that is specifically recognized by an antibody or other binding partner. The term also refers to the binding partner complex as well. Thus, for example, biotin or a biotin/avidin complex are both regarded as an affinity tag. In addition to epitopes recognized in epitope/antibody interactions, affinity tags also comprise “epitopes” recognized by other binding molecules (e.g. ligands bound by receptors), ligands bound by other ligands to form heterodimers or homodimers, His₆ bound by Ni-NTA, biotin bound by avidin, streptavidin, or anti-biotin antibodies, and the like.

Epitope tags are well known to those of skill in the art. Moreover, antibodies specific to a wide variety of epitope tags are commercially available. These include but are not limited to antibodies against the DYKDDDDK (SEQ ID NO:430) epitope, c-myc antibodies (available from Sigma, St. Louis), the HNK-1 carbohydrate epitope, the HA epitope, the HSV epitope, the His₄ (SEQ ID NO:431), His₅ (SEQ ID NO:432), and His₆ (SEQ ID NO:433) epitopes that are recognized by the His epitope specific antibodies (see, e.g., Qiagen), and the like.

Means of detecting such labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like. Similarly, enzymatic labels may be detected by providing appropriate substrates for the enzyme and detecting the resulting reaction product. Finally, simple calorimetric labels may be detected simply by observing the color associated with the label.

### Example 4

### Methods of Fine-Tuning the Identified Peptides in the Matrix to Obtain Peptides with Greater Specificity and Activity

Peptides identified in the initial binding screens as interacting with S. aureus cells were selected as the basis for a refined binding library. Because the peptides in row A of this pilot library have been seen to bind nonspecifically to a wide variety of bacterial surfaces (see FIG. 5), peptides C3, C4, D3, and D4 showed some of the brightest unique staining and were chosen to constitute the corners of the refined matrices (FIGS. 7A and 7B). Two refinements were developed: first, an “in-plane” matrix, in which the same parameters were varied as in the original pilot library, with hydrophobicity varying in the rows of the matrix and charge varying in the columns, but using smaller step sizes than in the original pilot library; and then an “orghogonal” matrix, in which hydrophobicity and charge vary according to one diagonal of the in-plane matrix and the periodicity of the hydrophobic and hydrophilic residues is varied. In the in-plane matrix (FIG. 7A), peptides C3, C4, D3, and D4 of the pilot library formed the corners of the refined matrix (C3 of the pilot library became A1 of the refined matrix, C4 became A4, D3 became D1). In the columns of the matrix, hydrophobicity varies from a column-averaged GRAVY=1.567 (column A) to 0.455 (column D) giving an average step size in this parameter of 0.34, compared to a difference of 1.012 in the original step. Though the net charge of peptides in this region of the original pilot library is zero, in the rows of the refined matrix, the pI of the peptides varies from 5.52 (row 1) to 6.07 (row 4) with an average step size of 0.18 compared to a difference of 0.55 in the original step. Therefore, the In-Plane refined matrix represents a more focused view of the region of the pilot library that gave rise to the original hits, allowing the effects of more subtle variations in hydrophobicity and charge to be explored.

In the orthogonal matrix (FIG. 7Bs), the diagonal of the in-plane matrix defined by In-Plane matrix peptides A1, B2, C3, and D4 was chosen to become row 3 of a 4×4 matrix. This allows limited variation in both hydrophobicity and charge according to the gradations established in the In-Plane matrix in the “columns” of the orthogonal matrix, where in the “rows” of this matrix, the periodicity with which the hydrophobic and hydrophilic peptides are placed is varied in a systematic fashion: from simple alternation of hydrophobic and hydrophilic residues (row 4), to alternation of two hydrophobic residues and two hydrophilic residues (the original periodicity of the pilot matrix, represented in row 3 of this orthogonal matrix), alternation of three hydrophobic residues and three hydrophilic residues (row 2), and finally, a partition of all hydrophobic residues to the amino terminal end of the peptide and all hydrophilic residues to the carboxy-terminal end (row 1). This allows exploration of different distributions of charged and hydrophobic regions in the peptide which may in turn lead to improved binding affinity or specificity within the confines of the parameter values determined to be effective at engendering surface binding (as defined by screening of the original matrix).

The peptides comprising these two refined libraries were synthesized, labeled, and screened against immobilized S. aureus cells exactly as for the original pilot library. Results are shown in FIGS. 7A and 7B: enhanced binding is seen for numerous peptides, including A3, B1, B2, B4, C1, C2, and C4 of the in-plane library and nearly all of the peptides in the orthogonal library, relative to that recorded for peptides in the original pilot library.

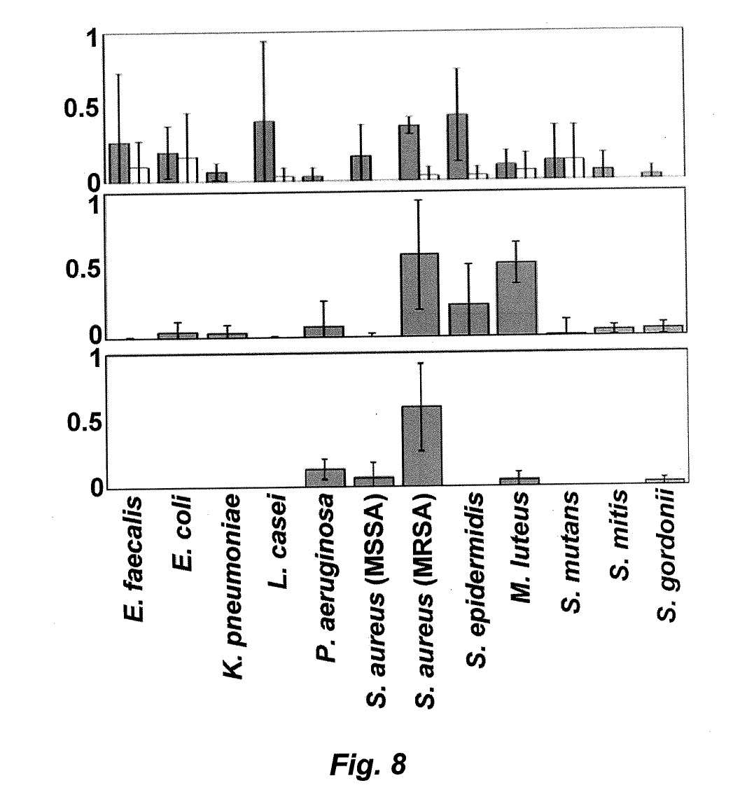

To examine the specificities of the peptides identified in this experiment, a diverse panel of various strains of bacteria was assembled, consisting of Lactobacillus casei, Escherichia coli, Enterococcus faecalis, Streptococcus mutans, Streptococcus mitis, Streptococcus gordonii, Pseudomonas aeruginosa, Klebsiella pneumoniae, Micrococcus luteus (strain MS2), Staphylococcus epidermidis, and two strains of Staphylococcus aureus: a methicillin/oxacillin-sensitive community isolate and our methicillin/oxacillin-resistant community isolate of (strain AM1). Peptides 3C and 4D from the original pilot library were screened for binding against these bacteria by fluorescence microscopy, and images were analyzed as before. Binding was clearly seen to both M. luteus and S. aureus, with significant levels of binding to several of the other bacteria tested (FIG. 8). Peptide 1B from the in-plane refined library was then screened, and as seen in FIG. 8, the strongest binding for this peptide is seen to M. luteus and S. aureus while no significant binding was seen to any of the other bacteria tested. Similarly, binding of peptide 3A from the in-plane refinement library was subjected to this panel, and binding was seen only to the methicillin/oxacilin-resistant strain of S. aureus.

To investigate whether the changes in the observed level of binding were due to changes in the affinities or avidities of the refined peptides for cell surfaces, binding curves were generated for selected peptides and fit to the Langmuir isotherm (where possible), the simplest model describing binding to surfaces (peptides showing either complex or nonspecific binding isotherms were excluded from this analysis).

Specifically, to determine their relative affinities for bacterial surfaces, selected peptides were subjected to pulldown assays as follows: Samples containing varying concentrations of fluorescein-labeled peptide (0-100 μM) and a fixed amount (approximately 7.5×10⁶ CFU) of bacterial cells were prepared. Measurements were taken of the absorbance of the labeled peptide at 488 nm (the peak absorbance of the fluorescein label) before and after exposure to the cells. The amount of peptide bound was calculated by comparing the ratio of the final (A_{f}) and initial (Aᵢ) absorbances to the initial concentration (C₀)[C_{bound}=C₀(1−(A_{f}/Aᵢ))]. Bacterial surface area per sample was calculated using a stage micrometer to determine the average cell diameter for each species (1.0+/−0.1 μm for S. aureus AM1, 2.7+/−0.1 μm for M. luteus MS2), treating individual cells as discrete spheres. Average cell concentration per unit OD₆₀₀ was calculated using a hemocytometer.

Plots were then generated of the amount of peptide bound per m² of bacterial surface area vs. the concentration of unbound peptide at equilibrium. Where possible, the resulting isotherms were fit to the Langmuir isotherm (P/A=(KₐNC_{eq})/(1+KₐC_{eq}), where P/A represents the molar amount of peptide bound per unit of bacterial surface area, Kₐ is the association constant of the peptide with the bacterial surface (L/Mol), N is the maximum surface concentration (mol/m²), and C_{eq} is the molar concentration of unbound peptide at equilibrium) (Calis et al. (1995) Pharm Res 12(7): 1072-1076). Data plots and curvefits were obtained using Kaleidagraph (Synergy Software).

Peptide 2A of the orthogonal refinement, for example, showed comparable affinity to the initial hits from the original pilot library, but a significant increase in Nₘₐₓ, indicating that its enhanced binding activity derives from a change in the maximum density of peptide molecules bound to the cell (Table 3). In addition, the increase in Nₘₐₓ seen for this peptide seems to be specific to its interaction with S. aureus, as no significant changes in either K_{A} or Nₘₐₓ were seen in its interaction with M. luteus relative to the peptides from the parent library. Alternatively, peptide 3A from the in-plane refinement, which showed some of the best specificity for S. aureus (see above) also shows increased affinity relative to the parent peptide, possibly explaining its increased binding activity, while its specificity is likely related to the sharp reduction in Nₘₐₓ on the M. luteus surface, indicating that binding to species other than S. aureus only occurs at very low densities. Other peptides show complex multistep binding isotherms or linear isotherms (indicating nonspecific binding), suggesting that, as predicted by our hypothesis, there exists a rich diversity of modes by which peptides may interact with a cell surface. While a few of these have been discussed here, the libraries generated in this study offer many opportunities for the further study of cell surface-peptide interactions.

TABLE 3
Surface binding affinity and monolayer concentrations for selected
peptides against S. aureus and M. luteus. Selected peptides are
those that showed Langmuir-type behavior when subjected to the
binding assay described in Methods.

| | | S. aureus | | M. luteus | |
|---|---|---|---|---|---|
| | | Langmuir | Monolayer | Langmuir | Monolayer |
| | | Affinity | Concentration | Affinity | Concentration |
| | | (Kₐ, M⁻¹) × | (N, Mol/m²) × | (Kₐ, M⁻¹) × | (N, Mol/m²) × |
| | Peptide | 10000 | 0.0001 | 10000 | 0.0001 |
| Pilot | 3C | 5.6 ± 0.9 | 0.31 ± 0.2 | 4 ± 1 | 0.12 ± 0.02 |
| Library | 3D | 6 ± 1 | 0.18 ± 0.2 | 10 ± 7 | 0.021 ± 0.004 |
| | 4C | 5 ± 1 | 0.23 ± 0.2 | 1 ± 0.6 | 0.10 ± 0.04 |
| | 4D | 5 ± 3 | 0.12 ± 0.3 | 5 ± 2 | 0.16 ± 0.02 |
| In-plane | 1B | ND | | 2.4 ± 0.8 | 0.11 ± 0.02 |
| refinement | 3A | ND | | 8 ± 3 | 0.0047 ± 0.006 |
| Orthogonal | 2A | 13 ± 4 | 0.14 ± 0.01 | 1.6 ± 0.7 | 0.5 ± 0.2 |
| refinement | 2B | | | 0.4 ± 0.3 | 0.5 ± 0.3 |
| | 2C | 1.1 ± 0.3 | 7 ± 1 | 1.4 ± 0.4 | 0.12 ± 0.03 |

ND—Not Determined.

### Example 5

### General Conclusion

Identifying peptides that bind specifically to protein targets requires either large, diverse libraries or small, focused libraries based on detailed prior knowledge of the target. Microbial surfaces are composed of a complex mixture of lipids, polysaccharides, and proteins that is unique within each bacterial species and yet is rich in potential peptide binding sites¹¹. Thus, much smaller libraries are required to identify peptides that interact with these surfaces. We have demonstrated here that a library of as few as thirty-six peptides, when designed with a sufficient diversity of charge distributions, can be used to successfully identify lead peptides that actively bind to bacterial surfaces.

In part, certain embodiments of the sparse matrix peptide library were developed to include the widest possible array of charge distributions within a small library. It was hypothesized, and surprisingly demonstrated that significant binding activity against cell surfaces can be found in a very small library. However, this typically results in the level of species specificity of any given hit being relatively low. The sparse-matrix approach allows refinement of these hits by generating similar sequences in an ordered fashion, and thus, much higher levels of specificity can be achieved. By choosing two parameters and modifying each in a stepwise fashion in one dimension of a two-dimensional matrix, an array of peptide sequences is generated that contains a wide variety of combined properties. The directionality of the matrix immediately suggests a refinement mechanism, in that identification of a peptide with modest binding activity actually defines a set of peptide sequences, bounded by the sequences on either side of the “hit,” that are likely to bind with equal or greater activity. Including orthogonal refinement in this process allows other possible periodicities to be considered, allowing refinement of both the magnitude and the spatial arrangement of the charge distribution of the candidate peptides.

Ligand binding, at least in the well-studied case of binding to protein targets, involves the interplay of many factors. Among the first in any list of determinants of ligand binding lie electrostatics and the hydrophobic effect (for excellent examples, see, e.g., Katz (1997) Annu Rev Biophys Biomol Struct 26: 27-45). In addition, the bulk properties of bacterial surfaces can be viewed primarily as a combination of charge and hydrophobicity (Dickson and Koohmaraie (1989) Appl Environ Microbiol 55(4): 832-836; Zita and Hermansson (1997) FEMS Microbiol Lett 152(2): 299-306; Groenink et al. (1998) Antonie Van Leeuwenhoek 73(3): 279-288; Wilson et al. (2001) Proc. Natl. Acad. Sci., USA, 98(7): 3750-3755). These two parameters have long been considered to be among the dominant terms in determining the selectivity of antimicrobial peptides, a broad class of peptides whose activity is predominantly determined by their direct interaction with microbial surfaces (Tossi et al. (2000) Biopolymers 55(1): 4-30; Glukhov et al. (2005) J Biol Chem 280(40): 33960-33967). Thus, in the design of a small peptide library whose primary goal was to identify peptides that bind specifically to microbial surfaces, it made sense to explore the section of the parameter space defined by the relative hydrophobicity and charge of potentially amphipathic peptides.

The initial pilot library was thus designed to have the widest possible variety of charge states and charge distributions, within the practical constraints defined by the size of the peptides (9 residues), the size of the library (36 peptides) and the periodicity with which the two parameters would be alternated (positions 1, 2, 5, 6 and 9 hydrophobic, positions 3, 4, 7 and 8 hydrophilic). Preliminary screening of this library has led to the identification of peptides that bind, at least modestly, to a number of bacterial species (see FIG. 6). Refinement of this library to identify similar peptides with improved affinity and specificity was straightforward: sub-matrices were generated around the “hits” varying, in one case, hydrophobicity and charge in smaller steps, and, in the orthogonal case, the periodicity with which hydrophobic and hydrophilic residues were alternated.

Screening of the pilot library revealed peptides that bound moderately to both Micrococcus and Staphylococcus. The refined library was subsequently found to contain peptides that were species-specific, clearly distinguishing between S. aureus and M. luteus. These species-specific peptides came from distinct regions of the refined matrix, indicating the capability of this method to make fine distinctions between similar bacterial surfaces. Further, the species-specific peptides, by combining modest increases in both affinity and avidity for their respective bacterial surfaces, show significantly higher levels of binding than did the peptides from the original pilot library.

In addition to its usefulness in the identification of lead peptides for the development of species-specific or surface-specific binding peptides, the pilot library can also be used for the detection and identification of unknown bacterial strains. Because the library consists of peptides that vary greatly in their charge and hydrophobicity, it is likely that any given bacterium will show some level of interaction with at least one of the peptides. At the same time, the variety that exists among bacterial surfaces ensures that any single peptide will rarely show an identical level of binding to two different bacteria, and thus the relative level of binding of peptides within the library should provide a unique “fingerprint” by which bacterial species or strains may be identified. Microarray methods have been previously proposed for the identification of microorganisms, primarily utilizing arrays of antibodies against species-specific surface proteins (Duburcq et al. (2004) Bioconjug Chem 15(2): 307-316; Kreutzberger (2006) Appl. Microbiol Biotechnol., 70(4): 383-390). While this approach allows the robust and highly sensitive detection of well characterized pathogens, its usefulness is limited to the identification of pathogens that are well enough known to have had highly specific antibodies derived against them. Emerging pathogens or unusual strains are unlikely to be noticed by these methods. Fingerprinting methods, by comparison, rely on the differential interaction of compounds in a library with the desired target and have been demonstrated to differentiate between specific proteins using libraries of 100-1000 compounds (Tomizaki et al. (2005) Chembiochem 6(5): 782-799; Reddy and Kodadek (2005) Proc. Natl. Acad. Sci., USA, 102 (36): 12672-12677). The representative bacterial surface binding profiles presented here (FIG. 6) show that each bacterium does show a distinctive binding profile, and the development of a set of profiles for known bacteria will likely allow the development of this technique for the rapid identification of unknown and emerging bacterial strains. The case of M. xanthus is especially illuminating in this regard: the binding profile of the exopolysaccharide-deficient mutant is similar enough to that of the wildtype that it can be recognized as M. xanthus, but clearly shows distinctive features marking it as an unusual strain. Thus, fingerprinting methods based on the approach presented here allow greater flexibility and inclusiveness than antibody-based microarrays and will merit further development as diagnostic elements.

It is important to note that the usefulness of this sparse-matrix screening method is not limited to bacterial surfaces: for example, screening of the pilot library against mammalian (Chinese Hamster Ovary) and yeast (C. albicans) cells showed that distinctive patterns of peptide binding can be obtained from eukaryotic cells as well. Surprisingly, applying this method to the bioinorganic surface of a sectioned human tooth revealed the presence in the library of peptides that bind specifically to tooth enamel (dentin-binding activities were also detected, in separate areas of the library). This may reflect the fact that mineral surfaces, while composed of a relatively restricted set of features (such as charged regions, hydrophobic regions, and topological elements), present those features in such a way as to allow multiple peptide binding modes. That is, the conformational flexibility available to potential binding peptides allows them to select a subset of surface features for interaction, causing the peptide, rather than the surface, to determine the geometry of binding. This makes the problem of binding to bioinorganic surfaces accessible, in principle, to very small peptide libraries such as the one presented here. At the same time, while the collection of surface features allows a wide variety of peptide binding modes, it is limited enough that unique peptide-surface interactions are possible. Distinctions can thus be made even between such similar surfaces as the differing forms of hydroxyapatite present in dentin and enamel in the case shown here. It is intriguing that the peptide sequence identified in this experiment does not resemble known mineral binding motifs. Hydroxyapatite binding motifs, in particular, generally make use of repetitive sequences rich in Asp or Glu to interact with the exposed positively charged calcium ions present on the crystal surface (Harris et al. (2000) Bone 27(6): 795-802; Kawasaki et al. (2004) Proc. Natl. Acad. Sci., USA, 101(31): 11356-11361; Addadi et al. (2001) Z Kardiol 90 Suppl 3: 92-98; Fujisawa and Kuboki (1998) Eur. J. Oral Sci., 106 Suppl 1: 249-253; Dahlin et al. (1998) Eur. J. Oral Sci., 106 Suppl 1: 239-248). The identification of an uncharged tissue-specific mineral binding peptide opens up new possibilities in the consideration of mechanisms by which peptides may interact with inorganic surfaces. Extending this method to other surfaces may allow us to identify additional novel sequences to bind to mineral, polymer, or metal surfaces.

We recently proposed the development of species-specific antimicrobial peptides as a possible means of limiting the side effects of antibiotic therapy, reducing antibiotic-associated infections, and overcoming resistance to current antibiotics (Eckert et al. (2006) Antimicrob Agents Chemother 50(4): 1480-1488). These targeted antimicrobials consist of membrane-disrupting killing domains fused through a short linker to a discrete targeting domain which confers species specificity. While this design is both simple and remarkably effective, its potential is limited by the availability of species-specific targeting peptides. A number of approaches are available to identify such targeting peptides, but most require some detailed knowledge of the target species. The method we describe here identifies highly active binding peptides without reference to any specific understanding of the target organism. This has the added advantage that it allows the rapid development of lead compounds to target new and emerging pathogens: in the example given here, peptides were identified to specifically target our test strains (S. aureus AM1 and M. luteus MS2) within days of their isolation. Further, the degree of specificity can be tuned, identifying peptides that target broader groups of bacteria (such as peptide 3C of the original pilot library, which binds well to both S. aureus and M. luteus) or peptides that show specificity at the species or even the strain level.

The ability to identify compounds that bind to specific surfaces is central to the development of therapeutics, diagnostics, and imaging agents that can target bacterial surfaces, mineralized tissues, and implanted devices. The sparse-matrix method described here places the means of producing and identifying these compounds well within the reach of modern high capacity peptide synthesizers. By utilizing simple free-solution screening methods, highly specific surface-binding peptides can be identified even in modestly equipped laboratories without the artifacts, complex deconvolution schemes, or high-throughput screening equipment required by large random libraries. By providing a simple and rapid means of developing peptides that specifically bind to desired surfaces, this method marks a step forward in the development of rapid diagnostics and targeted therapies.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A multidimensional sparse matrix library of compounds said library comprising:
a dimensionality D where D is the number of different compound properties systematically varied in said library and D ranges from about 2 to about 10;
wherein compounds comprising said library are characterized by different properties P₁, P₂, through P_{D} and each compound comprising said library can be designated as C_{P1, P2, P3, . . . PD}, such that compounds designated with the same subscripts P₁, P₂, through P_{D} have essentially the same values for the properties identified with those subscripts and vary with respect to the remaining properties, and wherein said library comprises no more than about 100 members that differ with respect to each property.

2. The sparse matrix library of claim 1, wherein said library is a 2-dimensional library (D=2) and where said library comprises:
an m×n matrix of a plurality of compounds,
wherein m and n are integers, 2≦m≦100, and 2≦n≦100;
wherein compounds comprising said library are characterized by a first property and a second property and can be designated as C_{i,j}, where 1≦i≦m, and 1≦j≦n, and compounds having the same value i and different values j have essentially the same value of the first property and vary with respect to the second property, while compounds having the same value j and different values i have essentially the same value of the second property and vary with respect to the first property.

3. The sparse matrix library of claim 2, wherein said compounds are polymeric biomolecules, but are not nucleic acids.

4. The sparse matrix library of claim 2, wherein said compounds are selected from the group consisting of polysaccharides, lipid polymers, and peptides and wherein said compounds are linear polymers, peptides and/or poptoids, and natural peptides.

5.-7. (canceled)

8. The sparse matrix library of claim 6, wherein the first property and second property are different properties and are independently selected from the group consisting of hydrophobicity, charge, length, periodicity, molecular weight, stokes radius, van der Waals radius, conformational flexibility, araomaticity, aliphatic index, helix-forming potential in aqueous solution, helix-forming potential in hydrophobic environments, dipole moment, sheet forming potential, polarizability, or any ratio of these parameters.

9. The sparse matrix library of claim 6, wherein the first property is hydrophobicity, and the second property is charge.

10. The sparse matrix library of claim 6, wherein the peptide and/or peptoids in said library comprises about 4 to about 50 amino acids.

11. The sparse matrix library of claim 6, wherein the peptide and/or peptoids in said library comprises about 4 to about 20 amino acids.

12. The sparse matrix library of claim 1, wherein said library comprises about 100 or fewer different compounds.

13. The sparse matrix library of claim 2, wherein the m×n matrix comprises less than about 100 different compounds.

14. The sparse matrix library of claim 2, wherein 2≦m≦100, 2≦m≦90, 2≦m≦80, 2≦m≦70, 2≦m≦60, 2≦m≦50, 2≦m≦40, 2≦m≦30, 2≦m≦20, or 2≦m≦10.

15. The sparse matrix library of claim 14, wherein m is 10, 9, 8, 7, 6, 5, 4, 3, or 2.

16. The sparse matrix library of claim 2, wherein 2≦n≦100, 2≦n≦90, 2≦n≦80, 2≦n≦70, 2≦n≦60, 2≦n≦50, 2≦n≦40, 2≦n≦30, 2≦n≦20, or 2≦n≦10.

17. The sparse matrix library of claim 16, wherein n is 10, 9, 8, 7, 6, 5, 4, 3, or 2.

18. The sparse matrix library according to claim 1, wherein the members of said library are labeled with a detectable label.

19. The sparse matrix library of claim 18, wherein said detectable label is selected from the group consisting of a radiometric label, a spin label, a fluorescent label, a calorimetric label, an enzymatic label, a quantum dot, a nanoparticle, and a magnetic label.

20. The sparse matrix library according to claim 1, wherein the members of said library are attached to a solid substrate.

21. The sparse matrix library according claim 20, wherein the members of said library are disposed on a contiguous solid substrate.

22. The sparse matrix library according claim 21, wherein the members of said library are spatially addressed so the identity of a member of the library can be determined by its location on said substrate.

23. The sparse matrix library according claim 20, wherein the members of said library are disposed on different substrates.

24. The sparse matrix library according to claim 1, wherein different members of the library are disposed in different containers or receptacles.

25. The sparse matrix library according to claim 24, wherein different members of the library are disposed in different wells of one or more microtiter plates.

26. The sparse matrix library according to claim 1, wherein different members of said library are disposed in containers containing cultures of cells, bacteria, protozoa, fungi, algae, or virus, wherein different containers contain different members of the library.

27.-40. (canceled)

41. A method of preparing a m×n peptide matrix,
wherein the matrix comprises m×n peptides,
wherein m and n are integers, 2≦m≦100, and 2≦n≦100;
wherein one said peptide in the matrix is designated as P_{i,j}, 1≦i≦m, and 1≦j≦n;
wherein each said peptide P_{i,j} is characterized by having a first parameter and a second parameter;
wherein a column of said peptides along the longitudinal direction in the matrix differ with each other in the value of the first parameter; and
wherein a row of said peptides along the latitudinal direction differ with each other the value of the second parameter; said method comprising:
i) providing a length of the peptides;
ii) providing a desired scope of the first parameter and the second parameter to be sampled;
iii) providing a first step size for the first parameter and a second step size for the second parameter or providing a size of the matrix;
iv) providing at least a first peptide possessing a first value for the first parameter and a second value for the second parameter; and
v) changing, substituting, or alternating the amino acids from the first peptide such that the peptides along the longitudinal direction in the matrix differ with each other in the value of the first parameter and the peptides along the latitudinal direction differ with each other the value of the second parameter.

42. The method of claim 41, further comprising providing a second peptide, a third peptide, and a fourth peptide:
wherein the first peptide is p_{1,1} possessing the first value which is a high (or low) end value of the first parameter and the second value which is a high (or low) end value of a second parameter,
wherein the second peptide is p_{m,1} possessing the low (or high) end value of the first parameter and the high (or low) end value of the second parameter,
wherein the third peptide is p_{1,n} possessing the high (or low) end value of the first parameter and the low (or high) end value of the second parameter, and
wherein the fourth peptide is p_{m,n} possessing the low (or high) end value of the first parameter and the low (of high) end value of the second parameter.

43.-50. (canceled)

51. A method of identifying a compound that specifically interacts with a target, said method comprising:
i) providing a sparse matrix library according to claim 1;
ii) contacting the compounds comprising the matrix to a target; and
iii) identifying and/or selecting the compounds that specifically and/or preferentially bind to the target.

52. The method of claim 51 further comprising a step of generating a second matrix using compounds selected or identified as specifically or preferentially binding said target.

53. The method of claim 52, wherein the target is selected from the group consisting of a biological surface, a microorganism, a prokaryotic cell, a eukaryotic cell, a receptor, and a compound, a yeast, a bacterium, a virus, a fungus, a protozoan, and an alga.

54. (canceled)

55. A method of identifying a pattern of interaction between a target and a peptide matrix library comprising the steps of:
i) providing a sparse matrix library according to claim 1;
ii) contacting the matrix members with a target; and
iii) recording the interaction of the target with each peptide in the matrix, wherein the collective interactions of the target with the matrix forms a fingerprint of the target with the peptide matrix.

56. A method of identifying a target to be tested comprising the steps of:
i) contacting the target to be tested with members of a sparse matrix library according to claim 1;
ii) recording a pattern of interaction of the target to be tested;
iii) comparing the pattern of interaction of the target and the library members to a database previously determined patterns of interaction for one or more known targets; and
iv) determining that the target to be tested is or is not one or said one or more targets.

57. A method of determine whether a member of a sparse matrix competes the binding of a compound to a target comprising the steps of
i) providing a sparse matrix library according to claim 1;
ii) contacting a target with the members of said library in the presence of a compound, wherein the compound binds an epitope of the target;
iii) measuring the level of interaction between library member(s) and the target and between the compound and the target, wherein the increasing interaction of the member(s) and the target and decreasing interaction of the target and the compound indicates that a compound in the matrix competes the binding of the compound to the target through the same or similar or overlapping epitope.

58. A composition comprising a peptide comprising the amino acid sequence AMKDAMERM (SEQ ID NO:429) wherein said peptide bind hydroxyapatite.

59. The composition of claim 58, wherein said peptide is labeled with a detectable label.

60. The composition of claim 59, wherein said detectable label is selected from the group consisting of a radio-opaque label, a radioactive label, an MRI contrast agent, a fluorescent label, a colorimetric label, and an epitope tag.


SEQUENCE LISTING (434 sequences)

SEQ ID NO: 1 - protein, 9 aa, Artificial Sequence
  synthetic amino acid sequence of peptide retro sequence
WWWWWEEEE

SEQ ID NO: 2 - protein, 9 aa, Artificial Sequence
  synthetic amino acid sequence of peptide
EEEEWWWWW

SEQ ID NO: 3 - protein, 8 aa, Artificial Sequence
  synthetic angiotensin II natural peptide
DRVYIHPF

SEQ ID NO: 4 - protein, 8 aa, Artificial Sequence
  synthetic peptide (peptoid) corresponding to angiotensin II
  MOD_RES (3)...(3): Xaa = N-(prop-2-yl)glycine
  MOD_RES (5)...(5): Xaa = N-(1-methylprop-1-yl)glycine
DRXYXHPF

SEQ ID NO: 5 - protein, 9 aa, Artificial Sequence
  synthetic bradykinin natural peptide
RPPGFSPFR

SEQ ID NO: 6 - protein, 9 aa, Artificial Sequence
  synthetic peptide (peptoid) corresponding to bradykinin
  MOD_RES (5)...(5): Xaa = N-benzylglycine
  MOD_RES (6)...(6): Xaa = N-(2-hydroxyethyl)glycine
  MOD_RES (8)...(8): Xaa = N-benzylglycine
RPPGXXPXR

SEQ ID NO: 7 - protein, 15 aa, Artificial Sequence
  synthetic beta-endorphin natural peptide
GGFMTSEKSQTPLVT

SEQ ID NO: 8 - protein, 15 aa, Artificial Sequence
  synthetic peptide (peptoid) corresponding to beta-endorphin
  MOD_RES (3)...(3): Xaa = N-benzylglycine
  MOD_RES (5)...(5): Xaa = N-(2-hydroxyethyl)glycine
  MOD_RES (8)...(8): Xaa = N-(aminoalkyl)glycine
  MOD_RES (11)...(11): Xaa = N-(2-hydroxyethyl)glycine
  MOD_RES (14)...(14): Xaa = N-(prop-2-yl)glycine
GGXMXSEXSQXPLXT

SEQ ID NO: 9 - protein, 9 aa, Artificial Sequence
  synthetic pilot library peptide A1
WWKHWWHRI

SEQ ID NO: 10 - protein, 9 aa, Artificial Sequence
  synthetic pilot library peptide A2
WWHSWWSTW

SEQ ID NO: 11 - protein, 9 aa, Artificial Sequence
  synthetic pilot library peptide A3
WWSYWWTQW

SEQ ID NO: 12 - protein, 9 aa, Artificial Sequence
  synthetic pilot library peptide A4
WWKDWWERW

SEQ ID NO: 13 - protein, 9 aa, Artificial Sequence
  synthetic pilot library peptide A5
WWYNWWQDW

SEQ ID NO: 14 - protein, 9 aa, Artificial Sequence
  synthetic pilot library peptide A6
WWQDWWNEW

SEQ ID NO: 15 - protein, 9 aa, Artificial Sequence
  synthetic pilot library peptide B1
FIKHFIHRF

SEQ ID NO: 16 - protein, 9 aa, Artificial Sequence
  synthetic pilot library peptide B2
FIHSFISTF

SEQ ID NO: 17 - protein, 9 aa, Artificial Sequence
  synthetic pilot library peptide B3
FISYFITQF

SEQ ID NO: 18 - protein, 9 aa, Artificial Sequence
  synthetic pilot library peptide B4
FIKDFIERF

SEQ ID NO: 19 - protein, 9 aa, Artificial Sequence
  synthetic pilot library peptide B5
FIYNFIQDF

SEQ ID NO: 20 - protein, 9 aa, Artificial Sequence
  synthetic pilot library peptide B6
FIQDFINEF

SEQ ID NO: 21 - protein, 9 aa, Artificial Sequence
  synthetic pilot library peptide C1
LIKHILHRL

SEQ ID NO: 22 - protein, 9 aa, Artificial Sequence
  synthetic pilot library peptide C2
LIHSILSTL

SEQ ID NO: 23 - protein, 9 aa, Artificial Sequence
  synthetic pilot library peptide C3
LISYILTQL

SEQ ID NO: 24 - protein, 9 aa, Artificial Sequence
  synthetic pilot library peptide C4
LIKDILERL

SEQ ID NO: 25 - protein, 9 aa, Artificial Sequence
  synthetic pilot library peptide C5
LIYNILQDL

SEQ ID NO: 26 - protein, 9 aa, Artificial Sequence
  synthetic pilot library peptide C6
LIQDILNEL

SEQ ID NO: 27 - protein, 9 aa, Artificial Sequence
  synthetic pilot library peptide D1
VAKHVAHRV

SEQ ID NO: 28 - protein, 9 aa, Artificial Sequence
  synthetic pilot library peptide D2
VAHSVASTV

SEQ ID NO: 29 - protein, 9 aa, Artificial Sequence
  synthetic pilot library peptide D3
VASYVATQV

SEQ ID NO: 30 - protein, 9 aa, Artificial Sequence
  synthetic pilot library peptide D4
VAKDVAERV

SEQ ID NO: 31 - protein, 9 aa, Artificial Sequence
  synthetic pilot library peptide D5
VAYNVAQDV

SEQ ID NO: 32 - protein, 9 aa, Artificial Sequence
  synthetic pilot library peptide D6
VAQDVANEV

SEQ ID NO: 33 - protein, 9 aa, Artificial Sequence
  synthetic pilot library peptide E1
AMKHAMHRM

SEQ ID NO: 34 - protein, 9 aa, Artificial Sequence
  synthetic pilot library peptide E2
AMHSAMSTM

SEQ ID NO: 35 - protein, 9 aa, Artificial Sequence
  synthetic pilot library peptide E3
AMSYAMTQM

SEQ ID NO: 36 - protein, 9 aa, Artificial Sequence
  synthetic pilot library peptide E4
AMKDAMERM

SEQ ID NO: 37 - protein, 9 aa, Artificial Sequence
  synthetic pilot library peptide E5
AMYNAMQDM

SEQ ID NO: 38 - protein, 9 aa, Artificial Sequence
  synthetic pilot library peptide E6
AMQDAMNEM

SEQ ID NO: 39 - protein, 9 aa, Artificial Sequence
  synthetic pilot library peptide F1
AAKHAAHRA

SEQ ID NO: 40 - protein, 9 aa, Artificial Sequence
  synthetic pilot library peptide F2
AAHSAASTA

SEQ ID NO: 41 - protein, 9 aa, Artificial Sequence
  synthetic pilot library peptide F3
AASYAATQA

SEQ ID NO: 42 - protein, 9 aa, Artificial Sequence
  synthetic pilot library peptide F4
AAKDAAERA

SEQ ID NO: 43 - protein, 9 aa, Artificial Sequence
  synthetic pilot library peptide F5
AAYNAAQDA

SEQ ID NO: 44 - protein, 9 aa, Artificial Sequence
  synthetic pilot library peptide F6
AAQDAANEA

SEQ ID NO: 45 - skipped

SEQ ID NO: 46 - protein, 9 aa, Artificial Sequence
  synthetic in-plane refinement library peptide A1
LISYILTQL

SEQ ID NO: 47 - protein, 9 aa, Artificial Sequence
  synthetic in-plane refinement library peptide A2
LISQILQNL

SEQ ID NO: 48 - protein, 9 aa, Artificial Sequence
  synthetic in-plane refinement library peptide A3
LIERILQNL

SEQ ID NO: 49 - protein, 9 aa, Artificial Sequence
  synthetic in-plane refinement library peptide A4
LIKDILERL

SEQ ID NO: 50 - protein, 9 aa, Artificial Sequence
  synthetic in-plane refinement library peptide B1
IVSYVITQL

SEQ ID NO: 51 - protein, 9 aa, Artificial Sequence
  synthetic in-plane refinement library peptide B2
IVSQVIQNL

SEQ ID NO: 52 - protein, 9 aa, Artificial Sequence
  synthetic in-plane refinement library peptide B3
IVERVIQNL

SEQ ID NO: 53 - protein, 9 aa, Artificial Sequence
  synthetic in-plane refinement library peptide B4
IVKDVIERL

SEQ ID NO: 54 - protein, 9 aa, Artificial Sequence
  synthetic in-plane refinement library peptide C1
LVSYVLTQV

SEQ ID NO: 55 - protein, 9 aa, Artificial Sequence
  synthetic in-plane refinement library peptide C2
LVSQVLQNV

SEQ ID NO: 56 - protein, 9 aa, Artificial Sequence
  synthetic in-plane refinement library peptide C3
LVERVLQNV

SEQ ID NO: 57 - protein, 9 aa, Artificial Sequence
  synthetic in-plane refinement library peptide C4
LVKDVLERV

SEQ ID NO: 58 - protein, 9 aa, Artificial Sequence
  synthetic in-plane refinement library peptide D1
VASYVATQV

SEQ ID NO: 59 - protein, 9 aa, Artificial Sequence
  synthetic in-plane refinement library peptide D2
VASQVAQNV

SEQ ID NO: 60 - protein, 9 aa, Artificial Sequence
  synthetic in-plane refinement library peptide D3
VAERVAQNV

SEQ ID NO: 61 - protein, 9 aa, Artificial Sequence
  synthetic in-plane refinement library peptide D4
VAKDVAERV

SEQ ID NO: 62 - protein, 9 aa, Artificial Sequence
  synthetic orthogonal refinement library peptide A1
LIILLSYTQ

SEQ ID NO: 63 - protein, 9 aa, Artificial Sequence
  synthetic orthogonal refinement library peptide A2
IVVILSQQN

SEQ ID NO: 64 - protein, 9 aa, Artificial Sequence
  synthetic orthogonal refinement library peptide A3
LVVLVERQN

SEQ ID NO: 65 - protein, 9 aa, Artificial Sequence
  synthetic orthogonal refinement library peptide A4
VAVAVKDER

SEQ ID NO: 66 - protein, 9 aa, Artificial Sequence
  synthetic orthogonal refinement library peptide B1
LIISYTLLQ

SEQ ID NO: 67 - protein, 9 aa, Artificial Sequence
  synthetic orthogonal refinement library peptide B2
IVVSQQILN

SEQ ID NO: 68 - protein, 9 aa, Artificial Sequence
  synthetic orthogonal refinement library peptide B3
LVVERQLVN

SEQ ID NO: 69 - protein, 9 aa, Artificial Sequence
  synthetic orthogonal refinement library peptide B4
VAVKDEAVR

SEQ ID NO: 70 - protein, 9 aa, Artificial Sequence
  synthetic orthogonal refinement library peptide C1
LISYILTQL

SEQ ID NO: 71 - protein, 9 aa, Artificial Sequence
  synthetic orthogonal refinement library peptide C2
IVSQVIQNL

SEQ ID NO: 72 - protein, 9 aa, Artificial Sequence
  synthetic orthogonal refinement library peptide C3
LVERVLQNV

SEQ ID NO: 73 - protein, 9 aa, Artificial Sequence
  synthetic orthogonal refinement library peptide C4
VAKDVAERV

SEQ ID NO: 74 - protein, 9 aa, Artificial Sequence
  synthetic orthogonal refinement library peptide D1
LSIYITLQL

SEQ ID NO: 75 - protein, 9 aa, Artificial Sequence
  synthetic orthogonal refinement library peptide D2
ISVQVQINL

SEQ ID NO: 76 - protein, 9 aa, Artificial Sequence
  synthetic orthogonal refinement library peptide D3
LEVRVQLNV

SEQ ID NO: 77 - protein, 9 aa, Artificial Sequence
  synthetic orthogonal refinement library peptide D4
VKADVEARV

SEQ ID NO: 78 - protein, 9 aa, Artificial Sequence
  synthetic exemplary 9-mer first peptide from corner of matrix library
WWKHWWHRW

SEQ ID NO: 79 - protein, 9 aa, Artificial Sequence
  synthetic exemplary 9-mer peptide from matrix library varying in hydrophobicity and charge
WWWWWEEEE

SEQ ID NO: 80 - protein, 9 aa, Artificial Sequence
  synthetic exemplary 9-mer peptide from matrix library varying in hydrophobicity and charge
WWWWWRRRR

SEQ ID NO: 81 - protein, 9 aa, Artificial Sequence
  synthetic exemplary 9-mer peptide from matrix library varying in hydrophobicity and charge
AAAAAEEEE

SEQ ID NO: 82 - protein, 9 aa, Artificial Sequence
  synthetic exemplary 9-mer peptide from matrix library varying in hydrophobicity and charge
AAAAARRRR

SEQ ID NO: 83 - protein, 9 aa, Artificial Sequence
  synthetic exemplary 9-mer peptide from matrix library varying in hydrophobicity and charge
WWWWEWEEE

SEQ ID NO: 84 - protein, 9 aa, Artificial Sequence
  synthetic exemplary 9-mer peptide from matrix library varying in hydrophobicity and charge
WWWWRWRRR

SEQ ID NO: 85 - protein, 9 aa, Artificial Sequence
  synthetic exemplary 9-mer peptide from matrix library varying in hydrophobicity and charge
AAAAEAEEE

SEQ ID NO: 86 - protein, 9 aa, Artificial Sequence
  synthetic exemplary 9-mer peptide from matrix library varying in hydrophobicity and charge
AAAARARRR

SEQ ID NO: 87 - protein, 9 aa, Artificial Sequence
  synthetic exemplary 9-mer peptide from matrix library varying in hydrophobicity and charge
WWWEWWEEE

SEQ ID NO: 88 - protein, 9 aa, Artificial Sequence
  synthetic exemplary 9-mer peptide from matrix library varying in hydrophobicity and charge
WWWRWWRRR

SEQ ID NO: 89 - protein, 9 aa, Artificial Sequence
  synthetic exemplary 9-mer peptide from matrix library varying in hydrophobicity and charge
AAAEAAEEE

SEQ ID NO: 90 - protein, 9 aa, Artificial Sequence
  synthetic exemplary 9-mer peptide from matrix library varying in hydrophobicity and charge
AAARAARRR

SEQ ID NO: 91 - protein, 9 aa, Artificial Sequence
  synthetic exemplary 9-mer peptide from matrix library varying in hydrophobicity and charge
WWEWWWEEE

SEQ ID NO: 92 - protein, 9 aa, Artificial Sequence
  synthetic exemplary 9-mer peptide from matrix library varying in hydrophobicity and charge
WWRWWWRRR

SEQ ID NO: 93 - protein, 9 aa, Artificial Sequence
  synthetic exemplary 9-mer peptide from matrix library varying in hydrophobicity and charge
AAEAAAEEE

SEQ ID NO: 94 - protein, 9 aa, Artificial Sequence
  synthetic exemplary 9-mer peptide from matrix library varying in hydrophobicity and charge
AARAAARRR

SEQ ID NO: 95 - protein, 9 aa, Artificial Sequence
  synthetic exemplary 9-mer peptide from matrix library varying in hydrophobicity and charge
WEWWWWEEE

SEQ ID NO: 96 - protein, 9 aa, Artificial Sequence
  synthetic exemplary 9-mer peptide from matrix library varying in hydrophobicity and charge
WRWWWWRRR

SEQ ID NO: 97 - protein, 9 aa, Artificial Sequence
  synthetic exemplary 9-mer peptide from matrix library varying in hydrophobicity and charge
AEAAAAEEE

SEQ ID NO: 98 - protein, 9 aa, Artificial Sequence
  synthetic exemplary 9-mer peptide from matrix library varying in hydrophobicity and charge
ARAAAARRR

SEQ ID NO: 99 - protein, 9 aa, Artificial Sequence
  synthetic exemplary 9-mer peptide from matrix library varying in hydrophobicity and charge
EWWWWWEEE

SEQ ID NO: 100 - protein, 9 aa, Artificial Sequence
  synthetic exemplary 9-mer peptide from matrix library varying in hydrophobicity and charge
RWWWWWRRR

SEQ ID NO: 101 - protein, 9 aa, Artificial Sequence
  synthetic exemplary 9-mer peptide from matrix library varying in hydrophobicity and charge
EAAAAAEEE

SEQ ID NO: 102 - protein, 9 aa, Artificial Sequence
  synthetic exemplary 9-mer peptide from matrix library varying in hydrophobicity and charge
RAAAAARRR

SEQ ID NO: 103 - protein, 9 aa, Artificial Sequence
  synthetic exemplary 9-mer peptide from matrix library varying in hydrophobicity and charge
EWWWWEWEE

SEQ ID NO: 104 - protein, 9 aa, Artificial Sequence
  synthetic exemplary 9-mer peptide from matrix library varying in hydrophobicity and charge
RWWWWRWRR

SEQ ID NO: 105 - protein, 9 aa, Artificial Sequence
  synthetic exemplary 9-mer peptide from matrix library varying in hydrophobicity and charge
EAAAAEAEE

SEQ ID NO: 106 - protein, 9 aa, Artificial Sequence
  synthetic exemplary 9-mer peptide from matrix library varying in hydrophobicity and charge
RAAAARARR

SEQ ID NO: 107 - protein, 9 aa, Artificial Sequence
  synthetic exemplary 9-mer peptide from matrix library varying in hydrophobicity and charge
EWWWEWWEE

SEQ ID NO: 108 - protein, 9 aa, Artificial Sequence
  synthetic exemplary 9-mer peptide from matrix library varying in hydrophobicity and charge
RWWWRWWRR

SEQ ID NO: 109 - protein, 9 aa, Artificial Sequence
  synthetic exemplary 9-mer peptide from matrix library varying in hydrophobicity and charge
EAAAEAAEE

SEQ ID NO: 110 - protein, 9 aa, Artificial Sequence
  synthetic exemplary 9-mer peptide from matrix library varying in hydrophobicity and charge
RAAARAARR

SEQ ID NO: 111 - protein, 9 aa, Artificial Sequence
  synthetic exemplary 9-mer peptide from matrix library varying in hydrophobicity and charge
EWWEWWWEE

SEQ ID NO: 112 - protein, 9 aa, Artificial Sequence
  synthetic exemplary 9-mer peptide from matrix library varying in hydrophobicity and charge
RWWRWWWRR

SEQ ID NO: 113 - protein, 9 aa, Artificial Sequence
  synthetic exemplary 9-mer peptide from matrix library varying in hydrophobicity and charge
EAAEAAAEE

SEQ ID NO: 114 - protein, 9 aa, Artificial Sequence
  synthetic exemplary 9-mer peptide from matrix library varying in hydrophobicity and charge
RAARAAARR

SEQ ID NO: 115 - protein, 9 aa, Artificial Sequence
  synthetic exemplary 9-mer peptide from matrix library varying in hydrophobicity and charge
EWEWWWWEE

SEQ ID NO: 116 - protein, 9 aa, Artificial Sequence
  synthetic exemplary 9-mer peptide from matrix library varying in hydrophobicity and charge
RWRWWWWRR

SEQ ID NO: 117 - protein, 9 aa, Artificial Sequence
  synthetic exemplary 9-mer peptide from matrix library varying in hydrophobicity and charge
EAEAAAAEE

SEQ ID NO: 118 - protein, 9 aa, Artificial Sequence
  synthetic exemplary 9-mer peptide from matrix library varying in hydrophobicity and charge
RARAAAARR

SEQ ID NO: 119 - protein, 9 aa, Artificial Sequence
  synthetic exemplary 9-mer peptide from matrix library varying in hydrophobicity and charge
EEWWWWWEE

SEQ ID NO: 120 - protein, 9 aa, Artificial Sequence
  synthetic exemplary 9-mer peptide from matrix library varying in hydrophobicity and charge
RRWWWWWRR

SEQ ID NO: 121 - protein, 9 aa, Artificial Sequence
  synthetic exemplary 9-mer peptide from matrix library varying in hydrophobicity and charge
EEAAAAAEE

SEQ ID NO: 122 - protein, 9 aa, Artificial Sequence
  synthetic exemplary 9-mer peptide from matrix library varying in hydrophobicity and charge
RRAAAAARR

SEQ ID NO: 123 - protein, 9 aa, Artificial Sequence
  synthetic exemplary 9-mer peptide from matrix library varying in hydrophobicity and charge
EEWWWWEWE

SEQ ID NO: 124 - protein, 9 aa, Artificial Sequence
  synthetic exemplary 9-mer peptide from matrix library varying in hydrophobicity and charge
RRWWWWRWR

SEQ ID NO: 125 - protein, 9 aa, Artificial Sequence
  synthetic exemplary 9-mer peptide from matrix library varying in hydrophobicity and charge
EEAAAAEAE

SEQ ID NO: 126 - protein, 9 aa, Artificial Sequence
  synthetic exemplary 9-mer peptide from matrix library varying in hydrophobicity and charge
RRAAAARAR

SEQ ID NO: 127 - protein, 9 aa, Artificial Sequence
  synthetic exemplary 9-mer peptide from matrix library varying in hydrophobicity and charge
EEWWWEWWE

SEQ ID NO: 128 - protein, 9 aa, Artificial Sequence
  synthetic exemplary 9-mer peptide from matrix library varying in hydrophobicity and charge
RRWWWRWWR

SEQ ID NO: 129 - protein, 9 aa, Artificial Sequence
  synthetic exemplary 9-mer peptide from matrix library varying in hydrophobicity and charge
EEAAAEAAE

SEQ ID NO: 130 - protein, 9 aa, Artificial Sequence
  synthetic exemplary 9-mer peptide from matrix library varying in hydrophobicity and charge
RRAAARAAR

SEQ ID NO: 131 - protein, 9 aa, Artificial Sequence
  synthetic exemplary 9-mer peptide from matrix library varying in hydrophobicity and charge
EEWWEWWWE

SEQ ID NO: 132 - protein, 9 aa, Artificial Sequence
  synthetic exemplary 9-mer peptide from matrix library varying in hydrophobicity and charge
RRWWRWWWR

SEQ ID NO: 133 - protein, 9 aa, Artificial Sequence
  synthetic exemplary 9-mer peptide from matrix library varying in hydrophobicity and charge
EEAAEAAAE

SEQ ID NO: 134 - protein, 9 aa, Artificial Sequence
  synthetic exemplary 9-mer peptide from matrix library varying in hydrophobicity and charge
RRAARAAAR

SEQ ID NO: 135 - protein, 9 aa, Artificial Sequence
  synthetic exemplary 9-mer peptide from matrix library varying in hydrophobicity and charge
EEWEWWWWE

SEQ ID NO: 136 - protein, 9 aa, Artificial Sequence
  synthetic exemplary 9-mer peptide from matrix library varying in hydrophobicity and charge
RRWRWWWWR

SEQ ID NO: 137 - protein, 9 aa, Artificial Sequence
  synthetic exemplary 9-mer peptide from matrix library varying in hydrophobicity and charge
EEAEAAAAE

SEQ ID NO: 138 - protein, 9 aa, Artificial Sequence
  synthetic exemplary 9-mer peptide from matrix library varying in hydrophobicity and charge
RRARAAAAR

SEQ ID NO: 139 - protein, 9 aa, Artificial Sequence
  synthetic exemplary 9-mer peptide from matrix library varying in hydrophobicity and charge
EEEWWWWWE

SEQ ID NO: 140 - protein, 9 aa, Artificial Sequence
  synthetic exemplary 9-mer peptide from matrix library varying in hydrophobicity and charge
RRRWWWWWR

SEQ ID NO: 141 - protein, 9 aa, Artificial Sequence
  synthetic exemplary 9-mer peptide from matrix library varying in hydrophobicity and charge
EEEAAAAAE

SEQ ID NO: 142 - protein, 9 aa, Artificial Sequence
  synthetic exemplary 9-mer peptide from matrix library varying in hydrophobicity and charge
RRRAAAAAR

SEQ ID NO: 143 - protein, 9 aa, Artificial Sequence
  synthetic exemplary 9-mer peptide from matrix library varying in hydrophobicity and charge
EEEWWWWEW

SEQ ID NO: 144 - protein, 9 aa, Artificial Sequence
  synthetic exemplary 9-mer peptide from matrix library varying in hydrophobicity and charge
RRRWWWWRW

SEQ ID NO: 145 - protein, 9 aa, Artificial Sequence
  synthetic exemplary 9-mer peptide from matrix library varying in hydrophobicity and charge
EEEAAAAEA

SEQ ID NO: 146 - protein, 9 aa, Artificial Sequence
  synthetic exemplary 9-mer peptide from matrix library varying in hydrophobicity and charge
RRRAAAARA

SEQ ID NO: 147 - protein, 9 aa, Artificial Sequence
  synthetic exemplary 9-mer peptide from matrix library varying in hydrophobicity and charge
EEEWWWEWW

SEQ ID NO: 148 - protein, 9 aa, Artificial Sequence
  synthetic exemplary 9-mer peptide from matrix library varying in hydrophobicity and charge
RRRWWWRWW

SEQ ID NO: 149 - protein, 9 aa, Artificial Sequence
  synthetic exemplary 9-mer peptide from matrix library varying in hydrophobicity and charge
EEEAAAEAA

SEQ ID NO: 150 - protein, 9 aa, Artificial Sequence
  synthetic exemplary 9-mer peptide from matrix library varying in hydrophobicity and charge
RRRAAARAA

SEQ ID NO: 151 - protein, 9 aa, Artificial Sequence
  synthetic exemplary 9-mer peptide from matrix library varying in hydrophobicity and charge
EEEWWEWWW

SEQ ID NO: 152 - protein, 9 aa, Artificial Sequence
  synthetic exemplary 9-mer peptide from matrix library varying in hydrophobicity and charge
RRRWWRWWW

SEQ ID NO: 153 - protein, 9 aa, Artificial Sequence
  synthetic exemplary 9-mer peptide from matrix library varying in hydrophobicity and charge
EEEAAEAAA

SEQ ID NO: 154 - protein, 9 aa, Artificial Sequence
  synthetic exemplary 9-mer peptide from matrix library varying in hydrophobicity and charge
RRRAARAAA

SEQ ID NO: 155 - protein, 9 aa, Artificial Sequence
  synthetic exemplary 9-mer peptide from matrix library varying in hydrophobicity and charge
EEEEWWWWW

SEQ ID NO: 156 - protein, 9 aa, Artificial Sequence
  synthetic exemplary 9-mer peptide from matrix library varying in hydrophobicity and charge
RRRRWWWWW

SEQ ID NO: 157 - protein, 9 aa, Artificial Sequence
  synthetic exemplary 9-mer peptide from matrix library varying in hydrophobicity and charge
EEEEAAAAA

SEQ ID NO: 158 - protein, 9 aa, Artificial Sequence
  synthetic exemplary 9-mer peptide from matrix library varying in hydrophobicity and charge
RRRRAAAAA

SEQ ID NO: 159 - protein, 9 aa, Artificial Sequence
  synthetic exemplary 9-mer peptide from matrix library varying in hydrophobicity and charge
WWWWEEWEE

SEQ ID NO: 160 - protein, 9 aa, Artificial Sequence
  synthetic exemplary 9-mer peptide from matrix library varying in hydrophobicity and charge
WWWWRRWRR

SEQ ID NO: 161 - protein, 9 aa, Artificial Sequence
  synthetic exemplary 9-mer peptide from matrix library varying in hydrophobicity and charge
AAAAEEAEE

SEQ ID NO: 162 - protein, 9 aa, Artificial Sequence
  synthetic exemplary 9-mer peptide from matrix library varying in hydrophobicity and charge
AAAARRARR

SEQ ID NO: 163 - protein, 9 aa, Artificial Sequence
  synthetic exemplary 9-mer peptide from matrix library varying in hydrophobicity and charge
WWWEEWWEE

SEQ ID NO: 164 - protein, 9 aa, Artificial Sequence
  synthetic exemplary 9-mer peptide from matrix library varying in hydrophobicity and charge
WWWRRWWRR

SEQ ID NO: 165 - protein, 9 aa, Artificial Sequence
  synthetic exemplary 9-mer peptide from matrix library varying in hydrophobicity and charge
AAAEEAAEE

SEQ ID NO: 166 - protein, 9 aa, Artificial Sequence
  synthetic exemplary 9-mer peptide from matrix library varying in hydrophobicity and charge
AAARRAARR

SEQ ID NO: 167 - protein, 9 aa, Artificial Sequence
  synthetic exemplary 9-mer peptide from matrix library varying in hydrophobicity and charge
WWEEWWWEE

SEQ ID NO: 168 - protein, 9 aa, Artificial Sequence
  synthetic exemplary 9-mer peptide from matrix library varying in hydrophobicity and charge
WWRRWWWRR

SEQ ID NO: 169 - protein, 9 aa, Artificial Sequence
  synthetic exemplary 9-mer peptide from matrix library varying in hydrophobicity and charge
AAEEAAAEE

SEQ ID NO: 170 - protein, 9 aa, Artificial Sequence
  synthetic exemplary 9-mer peptide from matrix library varying in hydrophobicity and charge
AARRAAARR

SEQ ID NO: 171 - protein, 9 aa, Artificial Sequence
  synthetic exemplary 9-mer peptide from matrix library varying in hydrophobicity and charge
WEEWWWWEE

SEQ ID NO: 172 - protein, 9 aa, Artificial Sequence
  synthetic exemplary 9-mer peptide from matrix library varying in hydrophobicity and charge
WRRWWWWRR

SEQ ID NO: 173 - protein, 9 aa, Artificial Sequence
  synthetic exemplary 9-mer peptide from matrix library varying in hydrophobicity and charge
AEEAAAAEE

SEQ ID NO: 174 - protein, 9 aa, Artificial Sequence
  synthetic exemplary 9-mer peptide from matrix library varying in hydrophobicity and charge
ARRAAAARR

SEQ ID NO: 175 - protein, 9 aa, Artificial Sequence
  synthetic exemplary 9-mer peptide from matrix library varying in hydrophobicity and charge
WWWWEEEWE

SEQ ID NO: 176 - protein, 9 aa, Artificial Sequence
  synthetic exemplary 9-mer peptide from matrix library varying in hydrophobicity and charge
WWWWRRRWR

SEQ ID NO: 177 - protein, 9 aa, Artificial Sequence
  synthetic exemplary 9-mer peptide from matrix library varying in hydrophobicity and charge
AAAAEEEAE

SEQ ID NO: 178 - protein, 9 aa, Artificial Sequence
  synthetic exemplary 9-mer peptide from matrix library varying in hydrophobicity and charge
AAAARRRAR

SEQ ID NO: 179 - protein, 9 aa, Artificial Sequence
  synthetic exemplary 9-mer peptide from matrix library varying in hydrophobicity and charge
WWWEEEWWE

SEQ ID NO: 180 - protein, 9 aa, Artificial Sequence
  synthetic exemplary 9-mer peptide from matrix library varying in hydrophobicity and charge
WWWRRRWWR

SEQ ID NO: 181 - protein, 9 aa, Artificial Sequence
  synthetic exemplary 9-mer peptide from matrix library varying in hydrophobicity and charge
AAAEEEAAE

SEQ ID NO: 182 - protein, 9 aa, Artificial Sequence
  synthetic exemplary 9-mer peptide from matrix library varying in hydrophobicity and charge
AAARRRAAR

SEQ ID NO: 183 - protein, 9 aa, Artificial Sequence
  synthetic exemplary 9-mer peptide from matrix library varying in hydrophobicity and charge
WWEEEWWWE

SEQ ID NO: 184 - protein, 9 aa, Artificial Sequence
  synthetic exemplary 9-mer peptide from matrix library varying in hydrophobicity and charge
WWRRRWWWR

SEQ ID NO: 185 - protein, 9 aa, Artificial Sequence
  synthetic exemplary 9-mer peptide from matrix library varying in hydrophobicity and charge
AAEEEAAAE

SEQ ID NO: 186 - protein, 9 aa, Artificial Sequence
  synthetic exemplary 9-mer peptide from matrix library varying in hydrophobicity and charge
AARRRAAAR

SEQ ID NO: 187 - protein, 9 aa, Artificial Sequence
  synthetic exemplary 9-mer peptide from matrix library varying in hydrophobicity and charge
WEEEWWWWE

SEQ ID NO: 188 - protein, 9 aa, Artificial Sequence
  synthetic exemplary 9-mer peptide from matrix library varying in hydrophobicity and charge
WRRRWWWWR

SEQ ID NO: 189 - protein, 9 aa, Artificial Sequence
  synthetic exemplary 9-mer peptide from matrix library varying in hydrophobicity and charge
AEEEAAAAE

SEQ ID NO: 190 - protein, 9 aa, Artificial Sequence
  synthetic exemplary 9-mer peptide from matrix library varying in hydrophobicity and charge
ARRRAAAAR

SEQ ID NO: 191 - protein, 9 aa, Artificial Sequence
  synthetic exemplary 9-mer peptide from matrix library varying in hydrophobicity and charge
EWWWWEEWE

SEQ ID NO: 192 - protein, 9 aa, Artificial Sequence
  synthetic exemplary 9-mer peptide from matrix library varying in hydrophobicity and charge
RWWWWRRWR

SEQ ID NO: 193 - protein, 9 aa, Artificial Sequence
  synthetic exemplary 9-mer peptide from matrix library varying in hydrophobicity and charge
EAAAAEEAE

SEQ ID NO: 194 - protein, 9 aa, Artificial Sequence
  synthetic exemplary 9-mer peptide from matrix library varying in hydrophobicity and charge
RAAAARRAR

SEQ ID NO: 195 - protein, 9 aa, Artificial Sequence
  synthetic exemplary 9-mer peptide from matrix library varying in hydrophobicity and charge
EWWWEEWWE

SEQ ID NO: 196 - protein, 9 aa, Artificial Sequence
  synthetic exemplary 9-mer peptide from matrix library varying in hydrophobicity and charge
RWWWRRWWR

SEQ ID NO: 197 - protein, 9 aa, Artificial Sequence
  synthetic exemplary 9-mer peptide from matrix library varying in hydrophobicity and charge
EAAAEEAAE

SEQ ID NO: 198 - protein, 9 aa, Artificial Sequence
  synthetic exemplary 9-mer peptide from matrix library varying in hydrophobicity and charge
RAAARRAAR

SEQ ID NO: 199 - protein, 9 aa, Artificial Sequence
  synthetic exemplary 9-mer peptide from matrix library varying in hydrophobicity and charge
EWWEEWWWE

SEQ ID NO: 200 - protein, 9 aa, Artificial Sequence
  synthetic exemplary 9-mer peptide from matrix library varying in hydrophobicity and charge
RWWRRWWWR

SEQ ID NO: 201 - protein, 9 aa, Artificial Sequence
  synthetic exemplary 9-mer peptide from matrix library varying in hydrophobicity and charge
EAAEEAAAE

SEQ ID NO: 202 - protein, 9 aa, Artificial Sequence
  synthetic exemplary 9-mer peptide from matrix library varying in hydrophobicity and charge
RAARRAAAR

SEQ ID NO: 203 - protein, 9 aa, Artificial Sequence
  synthetic exemplary 9-mer peptide from matrix library varying in hydrophobicity and charge
EWEEWWWWE

SEQ ID NO: 204 - protein, 9 aa, Artificial Sequence
  synthetic exemplary 9-mer peptide from matrix library varying in hydrophobicity and charge
RWRRWWWWR

SEQ ID NO: 205 - protein, 9 aa, Artificial Sequence
  synthetic exemplary 9-mer peptide from matrix library varying in hydrophobicity and charge
EAEEAAAAE

SEQ ID NO: 206 - protein, 9 aa, Artificial Sequence
  synthetic exemplary 9-mer peptide from matrix library varying in hydrophobicity and charge
RARRAAAAR

SEQ ID NO: 207 - protein, 9 aa, Artificial Sequence
  synthetic exemplary 9-mer peptide from matrix library varying in hydrophobicity and charge
EEWWWWEEW

SEQ ID NO: 208 - protein, 9 aa, Artificial Sequence
  synthetic exemplary 9-mer peptide from matrix library varying in hydrophobicity and charge
RRWWWWRRW

SEQ ID NO: 209 - protein, 9 aa, Artificial Sequence
  synthetic exemplary 9-mer peptide from matrix library varying in hydrophobicity and charge
EEAAAAEEA

SEQ ID NO: 210 - protein, 9 aa, Artificial Sequence
  synthetic exemplary 9-mer peptide from matrix library varying in hydrophobicity and charge
RRAAAARRA

SEQ ID NO: 211 - protein, 9 aa, Artificial Sequence
  synthetic exemplary 9-mer peptide from matrix library varying in hydrophobicity and charge
EEWWWEEWW

SEQ ID NO: 212 - protein, 9 aa, Artificial Sequence
  synthetic exemplary 9-mer peptide from matrix library varying in hydrophobicity and charge
RRWWWRRWW

SEQ ID NO: 213 - protein, 9 aa, Artificial Sequence
  synthetic exemplary 9-mer peptide from matrix library varying in hydrophobicity and charge
EEAAAEEAA

SEQ ID NO: 214 - protein, 9 aa, Artificial Sequence
  synthetic exemplary 9-mer peptide from matrix library varying in hydrophobicity and charge
RRAAARRAA

SEQ ID NO: 215 - protein, 9 aa, Artificial Sequence
  synthetic exemplary 9-mer peptide from matrix library varying in hydrophobicity and charge
EEWWEEWWW

SEQ ID NO: 216 - protein, 9 aa, Artificial Sequence
  synthetic exemplary 9-mer peptide from matrix library varying in hydrophobicity and charge
RRWWRRWWW

SEQ ID NO: 217 - protein, 9 aa, Artificial Sequence
  synthetic exemplary 9-mer peptide from matrix library varying in hydrophobicity and charge
EEAAEEAAA

SEQ ID NO: 218 - protein, 9 aa, Artificial Sequence
  synthetic exemplary 9-mer peptide from matrix library varying in hydrophobicity and charge
RRAARRAAA

SEQ ID NO: 219 - protein, 9 aa, Artificial Sequence
  synthetic exemplary 9-mer peptide from matrix library varying in hydrophobicity and charge
EEWEEWWWW

SEQ ID NO: 220 - protein, 9 aa, Artificial Sequence
  synthetic exemplary 9-mer peptide from matrix library varying in hydrophobicity and charge
RRWRRWWWW

SEQ ID NO: 221 - protein, 9 aa, Artificial Sequence
  synthetic exemplary 9-mer peptide from matrix library varying in hydrophobicity and charge
EEAEEAAAA

SEQ ID NO: 222 - protein, 9 aa, Artificial Sequence
  synthetic exemplary 9-mer peptide from matrix library varying in hydrophobicity and charge
RRARRAAAA

SEQ ID NO: 223 - protein, 9 aa, Artificial Sequence
  synthetic exemplary 9-mer peptide from matrix library varying in hydrophobicity and charge
EWWWWEEEW

SEQ ID NO: 224 - protein, 9 aa, Artificial Sequence
  synthetic exemplary 9-mer peptide from matrix library varying in hydrophobicity and charge
RWWWWRRRW

SEQ ID NO: 225 - protein, 9 aa, Artificial Sequence
  synthetic exemplary 9-mer peptide from matrix library varying in hydrophobicity and charge
EAAAAEEEA

SEQ ID NO: 226 - protein, 9 aa, Artificial Sequence
  synthetic exemplary 9-mer peptide from matrix library varying in hydrophobicity and charge
RAAAARRRA

SEQ ID NO: 227 - protein, 9 aa, Artificial Sequence
  synthetic exemplary 9-mer peptide from matrix library varying in hydrophobicity and charge
EWWWEEEWW

SEQ ID NO: 228 - protein, 9 aa, Artificial Sequence
  synthetic exemplary 9-mer peptide from matrix library varying in hydrophobicity and charge
RWWWRRRWW

SEQ ID NO: 229 - protein, 9 aa, Artificial Sequence
  synthetic exemplary 9-mer peptide from matrix library varying in hydrophobicity and charge
EAAAEEEAA

SEQ ID NO: 230 - protein, 9 aa, Artificial Sequence
  synthetic exemplary 9-mer peptide from matrix library varying in hydrophobicity and charge
RAAARRRAA

SEQ ID NO: 231 - protein, 9 aa, Artificial Sequence
  synthetic exemplary 9-mer peptide from matrix library varying in hydrophobicity and charge
EWWEEEWWW

SEQ ID NO: 232 - protein, 9 aa, Artificial Sequence
  synthetic exemplary 9-mer peptide from matrix library varying in hydrophobicity and charge
RWWRRRWWW

SEQ ID NO: 233 - protein, 9 aa, Artificial Sequence
  synthetic exemplary 9-mer peptide from matrix library varying in hydrophobicity and charge
EAAEEEAAA

SEQ ID NO: 234 - protein, 9 aa, Artificial Sequence
  synthetic exemplary 9-mer peptide from matrix library varying in hydrophobicity and charge
RAARRRAAA

SEQ ID NO: 235 - protein, 9 aa, Artificial Sequence
  synthetic exemplary 9-mer peptide from matrix library varying in hydrophobicity and charge
EWEEEWWWW

SEQ ID NO: 236 - protein, 9 aa, Artificial Sequence
  synthetic exemplary 9-mer peptide from matrix library varying in hydrophobicity and charge
RWRRRWWWW

SEQ ID NO: 237 - protein, 9 aa, Artificial Sequence
  synthetic exemplary 9-mer peptide from matrix library varying in hydrophobicity and charge
EAEEEAAAA

SEQ ID NO: 238 - protein, 9 aa, Artificial Sequence
  synthetic exemplary 9-mer peptide from matrix library varying in hydrophobicity and charge
RARRRAAAA

SEQ ID NO: 239 - protein, 9 aa, Artificial Sequence
  synthetic exemplary 9-mer peptide from matrix library varying in hydrophobicity and charge
WWWEWEWEE

SEQ ID NO: 240 - protein, 9 aa, Artificial Sequence
  synthetic exemplary 9-mer peptide from matrix library varying in hydrophobicity and charge
WWWRWRWRR

SEQ ID NO: 241 - protein, 9 aa, Artificial Sequence
  synthetic exemplary 9-mer peptide from matrix library varying in hydrophobicity and charge
AAAEAEAEE

SEQ ID NO: 242 - protein, 9 aa, Artificial Sequence
  synthetic exemplary 9-mer peptide from matrix library varying in hydrophobicity and charge
AAARARARR

SEQ ID NO: 243 - protein, 9 aa, Artificial Sequence
  synthetic exemplary 9-mer peptide from matrix library varying in hydrophobicity and charge
WWEWEWWEE

SEQ ID NO: 244 - protein, 9 aa, Artificial Sequence
  synthetic exemplary 9-mer peptide from matrix library varying in hydrophobicity and charge
WWRWRWWRR

SEQ ID NO: 245 - protein, 9 aa, Artificial Sequence
  synthetic exemplary 9-mer peptide from matrix library varying in hydrophobicity and charge
AAEAEAAEE

SEQ ID NO: 246 - protein, 9 aa, Artificial Sequence
  synthetic exemplary 9-mer peptide from matrix library varying in hydrophobicity and charge
AARARAARR

SEQ ID NO: 247 - protein, 9 aa, Artificial Sequence
  synthetic exemplary 9-mer peptide from matrix library varying in hydrophobicity and charge
WEWEWWWEE

SEQ ID NO: 248 - protein, 9 aa, Artificial Sequence
  synthetic exemplary 9-mer peptide from matrix library varying in hydrophobicity and charge
WRWRWWWRR

SEQ ID NO: 249 - protein, 9 aa, Artificial Sequence
  synthetic exemplary 9-mer peptide from matrix library varying in hydrophobicity and charge
AEAEAAAEE

SEQ ID NO: 250 - protein, 9 aa, Artificial Sequence
  synthetic exemplary 9-mer peptide from matrix library varying in hydrophobicity and charge
ARARAAARR

SEQ ID NO: 251 - protein, 9 aa, Artificial Sequence
  synthetic exemplary 9-mer peptide from matrix library varying in hydrophobicity and charge
EWEWWWWEE

SEQ ID NO: 252 - protein, 9 aa, Artificial Sequence
  synthetic exemplary 9-mer peptide from matrix library varying in hydrophobicity and charge
RWRWWWWRR

SEQ ID NO: 253 - protein, 9 aa, Artificial Sequence
  synthetic exemplary 9-mer peptide from matrix library varying in hydrophobicity and charge
EAEAAAAEE

SEQ ID NO: 254 - protein, 9 aa, Artificial Sequence
  synthetic exemplary 9-mer peptide from matrix library varying in hydrophobicity and charge
RARAAAARR

SEQ ID NO: 255 - protein, 9 aa, Artificial Sequence
  synthetic exemplary 9-mer peptide from matrix library varying in hydrophobicity and charge
EWWWEWEWE

SEQ ID NO: 256 - protein, 9 aa, Artificial Sequence
  synthetic exemplary 9-mer peptide from matrix library varying in hydrophobicity and charge
RWWWRWRWR

SEQ ID NO: 257 - protein, 9 aa, Artificial Sequence
  synthetic exemplary 9-mer peptide from matrix library varying in hydrophobicity and charge
EAAAEAEAE

SEQ ID NO: 258 - protein, 9 aa, Artificial Sequence
  synthetic exemplary 9-mer peptide from matrix library varying in hydrophobicity and charge
RAAARARAR

SEQ ID NO: 259 - protein, 9 aa, Artificial Sequence
  synthetic exemplary 9-mer peptide from matrix library varying in hydrophobicity and charge
EWWEWEWWE

SEQ ID NO: 260 - protein, 9 aa, Artificial Sequence
  synthetic exemplary 9-mer peptide from matrix library varying in hydrophobicity and charge
RWWRWRWWR

SEQ ID NO: 261 - protein, 9 aa, Artificial Sequence
  synthetic exemplary 9-mer peptide from matrix library varying in hydrophobicity and charge
EAAEAEAAE

SEQ ID NO: 262 - protein, 9 aa, Artificial Sequence
  synthetic exemplary 9-mer peptide from matrix library varying in hydrophobicity and charge
RAARARAAR

SEQ ID NO: 263 - protein, 9 aa, Artificial Sequence
  synthetic exemplary 9-mer peptide from matrix library varying in hydrophobicity and charge
EWEWEWWWE

SEQ ID NO: 264 - protein, 9 aa, Artificial Sequence
  synthetic exemplary 9-mer peptide from matrix library varying in hydrophobicity and charge
RWRWRWWWR

SEQ ID NO: 265 - protein, 9 aa, Artificial Sequence
  synthetic exemplary 9-mer peptide from matrix library varying in hydrophobicity and charge
EAEAEAAAE

SEQ ID NO: 266 - protein, 9 aa, Artificial Sequence
  synthetic exemplary 9-mer peptide from matrix library varying in hydrophobicity and charge
RARARAAAR

SEQ ID NO: 267 - protein, 9 aa, Artificial Sequence
  synthetic exemplary 9-mer peptide from matrix library varying in hydrophobicity and charge
EEWEWWWWE

SEQ ID NO: 268 - protein, 9 aa, Artificial Sequence
  synthetic exemplary 9-mer peptide from matrix library varying in hydrophobicity and charge
RRWRWWWWR

SEQ ID NO: 269 - protein, 9 aa, Artificial Sequence
  synthetic exemplary 9-mer peptide from matrix library varying in hydrophobicity and charge
EEAEAAAAE

SEQ ID NO: 270 - protein, 9 aa, Artificial Sequence
  synthetic exemplary 9-mer peptide from matrix library varying in hydrophobicity and charge
RRARAAAAR

SEQ ID NO: 271 - protein, 9 aa, Artificial Sequence
  synthetic exemplary 9-mer peptide from matrix library varying in hydrophobicity and charge
EEWWWEWEW

SEQ ID NO: 272 - protein, 9 aa, Artificial Sequence
  synthetic exemplary 9-mer peptide from matrix library varying in hydrophobicity and charge
RRWWWRWRW

SEQ ID NO: 273 - protein, 9 aa, Artificial Sequence
  synthetic exemplary 9-mer peptide from matrix library varying in hydrophobicity and charge
EEAAAEAEA

SEQ ID NO: 274 - protein, 9 aa, Artificial Sequence
  synthetic exemplary 9-mer peptide from matrix library varying in hydrophobicity and charge
RRAAARARA

SEQ ID NO: 275 - protein, 9 aa, Artificial Sequence
  synthetic exemplary 9-mer peptide from matrix library varying in hydrophobicity and charge
EEWWEWEWW

SEQ ID NO: 276 - protein, 9 aa, Artificial Sequence
  synthetic exemplary 9-mer peptide from matrix library varying in hydrophobicity and charge
RRWWRWRWW

SEQ ID NO: 277 - protein, 9 aa, Artificial Sequence
  synthetic exemplary 9-mer peptide from matrix library varying in hydrophobicity and charge
EEAAEAEAA

SEQ ID NO: 278 - protein, 9 aa, Artificial Sequence
  synthetic exemplary 9-mer peptide from matrix library varying in hydrophobicity and charge
RRAARARAA

SEQ ID NO: 279 - protein, 9 aa, Artificial Sequence
  synthetic exemplary 9-mer peptide from matrix library varying in hydrophobicity and charge
EEWEWEWWW

SEQ ID NO: 280 - protein, 9 aa, Artificial Sequence
  synthetic exemplary 9-mer peptide from matrix library varying in hydrophobicity and charge
RRWRWRWWW

SEQ ID NO: 281 - protein, 9 aa, Artificial Sequence
  synthetic exemplary 9-mer peptide from matrix library varying in hydrophobicity and charge
EEAEAEAAA

SEQ ID NO: 282 - protein, 9 aa, Artificial Sequence
  synthetic exemplary 9-mer peptide from matrix library varying in hydrophobicity and charge
RRARARAAA

SEQ ID NO: 283 - protein, 9 aa, Artificial Sequence
  synthetic exemplary 9-mer peptide from matrix library varying in hydrophobicity and charge
EEEWEWWWW

SEQ ID NO: 284 - protein, 9 aa, Artificial Sequence
  synthetic exemplary 9-mer peptide from matrix library varying in hydrophobicity and charge
RRRWRWWWW

SEQ ID NO: 285 - protein, 9 aa, Artificial Sequence
  synthetic exemplary 9-mer peptide from matrix library varying in hydrophobicity and charge
EEEAEAAAA

SEQ ID NO: 286 - protein, 9 aa, Artificial Sequence
  synthetic exemplary 9-mer peptide from matrix library varying in hydrophobicity and charge
RRRARAAAA

SEQ ID NO: 287 - protein, 9 aa, Artificial Sequence
  synthetic exemplary 9-mer peptide from matrix library varying in hydrophobicity and charge
WEWWEWWEE

SEQ ID NO: 288 - protein, 9 aa, Artificial Sequence
  synthetic exemplary 9-mer peptide from matrix library varying in hydrophobicity and charge
WRWWRWWRR

SEQ ID NO: 289 - protein, 9 aa, Artificial Sequence
  synthetic exemplary 9-mer peptide from matrix library varying in hydrophobicity and charge
AEAAEAAEE

SEQ ID NO: 290 - protein, 9 aa, Artificial Sequence
  synthetic exemplary 9-mer peptide from matrix library varying in hydrophobicity and charge
ARAARAARR

SEQ ID NO: 291 - protein, 9 aa, Artificial Sequence
  synthetic exemplary 9-mer peptide from matrix library varying in hydrophobicity and charge
EWWEWWWEE

SEQ ID NO: 292 - protein, 9 aa, Artificial Sequence
  synthetic exemplary 9-mer peptide from matrix library varying in hydrophobicity and charge
RWWRWWWRR

SEQ ID NO: 293 - protein, 9 aa, Artificial Sequence
  synthetic exemplary 9-mer peptide from matrix library varying in hydrophobicity and charge
EAAEAAAEE

SEQ ID NO: 294 - protein, 9 aa, Artificial Sequence
  synthetic exemplary 9-mer peptide from matrix library varying in hydrophobicity and charge
RAARAAARR

SEQ ID NO: 295 - protein, 9 aa, Artificial Sequence
  synthetic exemplary 9-mer peptide from matrix library varying in hydrophobicity and charge
EWEWWEWWE

SEQ ID NO: 296 - protein, 9 aa, Artificial Sequence
  synthetic exemplary 9-mer peptide from matrix library varying in hydrophobicity and charge
RWRWWRWWR

SEQ ID NO: 297 - protein, 9 aa, Artificial Sequence
  synthetic exemplary 9-mer peptide from matrix library varying in hydrophobicity and charge
EAEAAEAAE

SEQ ID NO: 298 - protein, 9 aa, Artificial Sequence
  synthetic exemplary 9-mer peptide from matrix library varying in hydrophobicity and charge
RARAARAAR

SEQ ID NO: 299 - protein, 9 aa, Artificial Sequence
  synthetic exemplary 9-mer peptide from matrix library varying in hydrophobicity and charge
EEWWEWWWE

SEQ ID NO: 300 - protein, 9 aa, Artificial Sequence
  synthetic exemplary 9-mer peptide from matrix library varying in hydrophobicity and charge
RRWWRWWWR

SEQ ID NO: 301 - protein, 9 aa, Artificial Sequence
  synthetic exemplary 9-mer peptide from matrix library varying in hydrophobicity and charge
EEAAEAAAE

SEQ ID NO: 302 - protein, 9 aa, Artificial Sequence
  synthetic exemplary 9-mer peptide from matrix library varying in hydrophobicity and charge
RRAARAAAR

SEQ ID NO: 303 - protein, 9 aa, Artificial Sequence
  synthetic exemplary 9-mer peptide from matrix library varying in hydrophobicity and charge
WWEEWWEEW

SEQ ID NO: 304 - protein, 9 aa, Artificial Sequence
  synthetic exemplary 9-mer peptide from matrix library varying in hydrophobicity and charge
WWRRWWRRW

SEQ ID NO: 305 - protein, 9 aa, Artificial Sequence
  synthetic exemplary 9-mer peptide from matrix library varying in hydrophobicity and charge
AAEEAAEEA

SEQ ID NO: 306 - protein, 9 aa, Artificial Sequence
  synthetic exemplary 9-mer peptide from matrix library varying in hydrophobicity and charge
AARRAARRA

SEQ ID NO: 307 - protein, 9 aa, Artificial Sequence
  synthetic exemplary 9-mer peptide from matrix library varying in hydrophobicity and charge
WEEWWEEWW

SEQ ID NO: 308 - protein, 9 aa, Artificial Sequence
  synthetic exemplary 9-mer peptide from matrix library varying in hydrophobicity and charge
WRRWWRRWW

SEQ ID NO: 309 - protein, 9 aa, Artificial Sequence
  synthetic exemplary 9-mer peptide from matrix library varying in hydrophobicity and charge
AEEAAEEAA

SEQ ID NO: 310 - protein, 9 aa, Artificial Sequence
  synthetic exemplary 9-mer peptide from matrix library varying in hydrophobicity and charge
ARRAARRAA

SEQ ID NO: 311 - protein, 9 aa, Artificial Sequence
  synthetic exemplary 9-mer peptide from matrix library varying in hydrophobicity and charge
EEWWEEWWW

SEQ ID NO: 312 - protein, 9 aa, Artificial Sequence
  synthetic exemplary 9-mer peptide from matrix library varying in hydrophobicity and charge
RRWWRRWWW

SEQ ID NO: 313 - protein, 9 aa, Artificial Sequence
  synthetic exemplary 9-mer peptide from matrix library varying in hydrophobicity and charge
EEAAEEAAA

SEQ ID NO: 314 - protein, 9 aa, Artificial Sequence
  synthetic exemplary 9-mer peptide from matrix library varying in hydrophobicity and charge
RRAARRAAA

SEQ ID NO: 315 - protein, 9 aa, Artificial Sequence
  synthetic exemplary 9-mer peptide from matrix library varying in hydrophobicity and charge
EWWEWWEEW

SEQ ID NO: 316 - protein, 9 aa, Artificial Sequence
  synthetic exemplary 9-mer peptide from matrix library varying in hydrophobicity and charge
RWWRWWRRW

SEQ ID NO: 317 - protein, 9 aa, Artificial Sequence
  synthetic exemplary 9-mer peptide from matrix library varying in hydrophobicity and charge
EAAEAAEEA

SEQ ID NO: 318 - protein, 9 aa, Artificial Sequence
  synthetic exemplary 9-mer peptide from matrix library varying in hydrophobicity and charge
RAARAARRA

SEQ ID NO: 319 - protein, 9 aa, Artificial Sequence
  synthetic exemplary 9-mer peptide from matrix library varying in hydrophobicity and charge
WWEWWEWEW

SEQ ID NO: 320 - protein, 9 aa, Artificial Sequence
  synthetic exemplary 9-mer peptide from matrix library varying in hydrophobicity and charge
WWRWWRWRW

SEQ ID NO: 321 - protein, 9 aa, Artificial Sequence
  synthetic exemplary 9-mer peptide from matrix library varying in hydrophobicity and charge
AAEAAEAEA

SEQ ID NO: 322 - protein, 9 aa, Artificial Sequence
  synthetic exemplary 9-mer peptide from matrix library varying in hydrophobicity and charge
AARAARARA

SEQ ID NO: 323 - protein, 9 aa, Artificial Sequence
  synthetic exemplary 9-mer peptide from matrix library varying in hydrophobicity and charge
EEWWEWWEW

SEQ ID NO: 324 - protein, 9 aa, Artificial Sequence
  synthetic exemplary 9-mer peptide from matrix library varying in hydrophobicity and charge
RRWWRWWRW

SEQ ID NO: 325 - protein, 9 aa, Artificial Sequence
  synthetic exemplary 9-mer peptide from matrix library varying in hydrophobicity and charge
EEAAEAAEA

SEQ ID NO: 326 - protein, 9 aa, Artificial Sequence
  synthetic exemplary 9-mer peptide from matrix library varying in hydrophobicity and charge
RRAARAARA

SEQ ID NO: 327 - protein, 9 aa, Artificial Sequence
  synthetic exemplary 9-mer peptide from matrix library varying in hydrophobicity and charge
EWEWWEEWW

SEQ ID NO: 328 - protein, 9 aa, Artificial Sequence
  synthetic exemplary 9-mer peptide from matrix library varying in hydrophobicity and charge
RWRWWRRWW

SEQ ID NO: 329 - protein, 9 aa, Artificial Sequence
  synthetic exemplary 9-mer peptide from matrix library varying in hydrophobicity and charge
EAEAAEEAA

SEQ ID NO: 330 - protein, 9 aa, Artificial Sequence
  synthetic exemplary 9-mer peptide from matrix library varying in hydrophobicity and charge
RARAARRAA

SEQ ID NO: 331 - protein, 9 aa, Artificial Sequence
  synthetic exemplary 9-mer peptide from matrix library varying in hydrophobicity and charge
EEWWEWEWW

SEQ ID NO: 332 - protein, 9 aa, Artificial Sequence
  synthetic exemplary 9-mer peptide from matrix library varying in hydrophobicity and charge
RRWWRWRWW

SEQ ID NO: 333 - protein, 9 aa, Artificial Sequence
  synthetic exemplary 9-mer peptide from matrix library varying in hydrophobicity and charge
EEAAEAEAA

SEQ ID NO: 334 - protein, 9 aa, Artificial Sequence
  synthetic exemplary 9-mer peptide from matrix library varying in hydrophobicity and charge
RRAARARAA

SEQ ID NO: 335 - protein, 9 aa, Artificial Sequence
  synthetic exemplary 9-mer peptide from matrix library varying in hydrophobicity and charge
WWEWEWEWE

SEQ ID NO: 336 - protein, 9 aa, Artificial Sequence
  synthetic exemplary 9-mer peptide from matrix library varying in hydrophobicity and charge
WWRWRWRWR

SEQ ID NO: 337 - protein, 9 aa, Artificial Sequence
  synthetic exemplary 9-mer peptide from matrix library varying in hydrophobicity and charge
AAEAEAEAE

SEQ ID NO: 338 - protein, 9 aa, Artificial Sequence
  synthetic exemplary 9-mer peptide from matrix library varying in hydrophobicity and charge
AARARARAR

SEQ ID NO: 339 - protein, 9 aa, Artificial Sequence
  synthetic exemplary 9-mer peptide from matrix library varying in hydrophobicity and charge
WEWEWEWEW

SEQ ID NO: 340 - protein, 9 aa, Artificial Sequence
  synthetic exemplary 9-mer peptide from matrix library varying in hydrophobicity and charge
WRWRWRWRW

SEQ ID NO: 341 - protein, 9 aa, Artificial Sequence
  synthetic exemplary 9-mer peptide from matrix library varying in hydrophobicity and charge
AEAEAEAEA

SEQ ID NO: 342 - protein, 9 aa, Artificial Sequence
  synthetic exemplary 9-mer peptide from matrix library varying in hydrophobicity and charge
ARARARARA

SEQ ID NO: 343 - protein, 9 aa, Artificial Sequence
  synthetic exemplary 9-mer peptide from matrix library varying in hydrophobicity and charge
EWEWEWEWW

SEQ ID NO: 344 - protein, 9 aa, Artificial Sequence
  synthetic exemplary 9-mer peptide from matrix library varying in hydrophobicity and charge
RWRWRWRWW

SEQ ID NO: 345 - protein, 9 aa, Artificial Sequence
  synthetic exemplary 9-mer peptide from matrix library varying in hydrophobicity and charge
EAEAEAEAA

SEQ ID NO: 346 - protein, 9 aa, Artificial Sequence
  synthetic exemplary 9-mer peptide from matrix library varying in hydrophobicity and charge
RARARARAA

SEQ ID NO: 347 - protein, 9 aa, Artificial Sequence
  synthetic exemplary 9-mer peptide from matrix library varying in hydrophobicity and charge
WEWEWWEEW

SEQ ID NO: 348 - protein, 9 aa, Artificial Sequence
  synthetic exemplary 9-mer peptide from matrix library varying in hydrophobicity and charge
WRWRWWRRW

SEQ ID NO: 349 - protein, 9 aa, Artificial Sequence
  synthetic exemplary 9-mer peptide from matrix library varying in hydrophobicity and charge
AEAEAAEEA

SEQ ID NO: 350 - protein, 9 aa, Artificial Sequence
  synthetic exemplary 9-mer peptide from matrix library varying in hydrophobicity and charge
ARARAARRA

SEQ ID NO: 351 - protein, 9 aa, Artificial Sequence
  synthetic exemplary 9-mer peptide from matrix library varying in hydrophobicity and charge
WEWEWEEWW

SEQ ID NO: 352 - protein, 9 aa, Artificial Sequence
  synthetic exemplary 9-mer peptide from matrix library varying in hydrophobicity and charge
WRWRWRRWW

SEQ ID NO: 353 - protein, 9 aa, Artificial Sequence
  synthetic exemplary 9-mer peptide from matrix library varying in hydrophobicity and charge
AEAEAEEAA

SEQ ID NO: 354 - protein, 9 aa, Artificial Sequence
  synthetic exemplary 9-mer peptide from matrix library varying in hydrophobicity and charge
ARARARRAA

SEQ ID NO: 355 - protein, 9 aa, Artificial Sequence
  synthetic exemplary 9-mer peptide from matrix library varying in hydrophobicity and charge
WEWEEEWWW

SEQ ID NO: 356 - protein, 9 aa, Artificial Sequence
  synthetic exemplary 9-mer peptide from matrix library varying in hydrophobicity and charge
WRWRRRWWW

SEQ ID NO: 357 - protein, 9 aa, Artificial Sequence
  synthetic exemplary 9-mer peptide from matrix library varying in hydrophobicity and charge
AEAEEEAAA

SEQ ID NO: 358 - protein, 9 aa, Artificial Sequence
  synthetic exemplary 9-mer peptide from matrix library varying in hydrophobicity and charge
ARARRRAAA

SEQ ID NO: 359 - protein, 9 aa, Artificial Sequence
  synthetic exemplary 9-mer peptide from matrix library varying in hydrophobicity and charge
WEEEEWWWW

SEQ ID NO: 360 - protein, 9 aa, Artificial Sequence
  synthetic exemplary 9-mer peptide from matrix library varying in hydrophobicity and charge
WRRRRWWWW

SEQ ID NO: 361 - protein, 9 aa, Artificial Sequence
  synthetic exemplary 9-mer peptide from matrix library varying in hydrophobicity and charge
AEEEEAAAA

SEQ ID NO: 362 - protein, 9 aa, Artificial Sequence
  synthetic exemplary 9-mer peptide from matrix library varying in hydrophobicity and charge
ARRRRAAAA

SEQ ID NO: 363 - protein, 9 aa, Artificial Sequence
  synthetic exemplary 9-mer peptide from matrix library varying in hydrophobicity and charge
WWEEEEWWW

SEQ ID NO: 364 - protein, 9 aa, Artificial Sequence
  synthetic exemplary 9-mer peptide from matrix library varying in hydrophobicity and charge
WWRRRRWWW

SEQ ID NO: 365 - protein, 9 aa, Artificial Sequence
  synthetic exemplary 9-mer peptide from matrix library varying in hydrophobicity and charge
AAEEEEAAA

SEQ ID NO: 366 - protein, 9 aa, Artificial Sequence
  synthetic exemplary 9-mer peptide from matrix library varying in hydrophobicity and charge
AARRRRAAA

SEQ ID NO: 367 - protein, 9 aa, Artificial Sequence
  synthetic exemplary 9-mer peptide from matrix library varying in hydrophobicity and charge
WWWEEEEWW

SEQ ID NO: 368 - protein, 9 aa, Artificial Sequence
  synthetic exemplary 9-mer peptide from matrix library varying in hydrophobicity and charge
WWWRRRRWW

SEQ ID NO: 369 - protein, 9 aa, Artificial Sequence
  synthetic exemplary 9-mer peptide from matrix library varying in hydrophobicity and charge
AAAEEEEAA

SEQ ID NO: 370 - protein, 9 aa, Artificial Sequence
  synthetic exemplary 9-mer peptide from matrix library varying in hydrophobicity and charge
AAARRRRAA

SEQ ID NO: 371 - protein, 9 aa, Artificial Sequence
  synthetic exemplary 9-mer peptide from matrix library varying in hydrophobicity and charge
WWWWEEEEW

SEQ ID NO: 372 - protein, 9 aa, Artificial Sequence
  synthetic exemplary 9-mer peptide from matrix library varying in hydrophobicity and charge
WWWWRRRRW

SEQ ID NO: 373 - protein, 9 aa, Artificial Sequence
  synthetic exemplary 9-mer peptide from matrix library varying in hydrophobicity and charge
AAAAEEEEA

SEQ ID NO: 374 - protein, 9 aa, Artificial Sequence
  synthetic exemplary 9-mer peptide from matrix library varying in hydrophobicity and charge
AAAARRRRA

SEQ ID NO: 375 - protein, 9 aa, Artificial Sequence
  synthetic exemplary 9-mer peptide from matrix library varying in hydrophobicity and charge
WWWEWEEEW

SEQ ID NO: 376 - protein, 9 aa, Artificial Sequence
  synthetic exemplary 9-mer peptide from matrix library varying in hydrophobicity and charge
WWWRWRRRW

SEQ ID NO: 377 - protein, 9 aa, Artificial Sequence
  synthetic exemplary 9-mer peptide from matrix library varying in hydrophobicity and charge
AAAEAEEEA

SEQ ID NO: 378 - protein, 9 aa, Artificial Sequence
  synthetic exemplary 9-mer peptide from matrix library varying in hydrophobicity and charge
AAARARRRA

SEQ ID NO: 379 - protein, 9 aa, Artificial Sequence
  synthetic exemplary 9-mer peptide from matrix library varying in hydrophobicity and charge
WEWEEEWWW

SEQ ID NO: 380 - protein, 9 aa, Artificial Sequence
  synthetic exemplary 9-mer peptide from matrix library varying in hydrophobicity and charge
WRWRRRWWW

SEQ ID NO: 381 - protein, 9 aa, Artificial Sequence
  synthetic exemplary 9-mer peptide from matrix library varying in hydrophobicity and charge
AEAEEEAAA

SEQ ID NO: 382 - protein, 9 aa, Artificial Sequence
  synthetic exemplary 9-mer peptide from matrix library varying in hydrophobicity and charge
ARARRRAAA

SEQ ID NO: 383 - protein, 9 aa, Artificial Sequence
  synthetic exemplary 9-mer peptide from matrix library varying in hydrophobicity and charge
WWWEEWEEW

SEQ ID NO: 384 - protein, 9 aa, Artificial Sequence
  synthetic exemplary 9-mer peptide from matrix library varying in hydrophobicity and charge
WWWRRWRRW

SEQ ID NO: 385 - protein, 9 aa, Artificial Sequence
  synthetic exemplary 9-mer peptide from matrix library varying in hydrophobicity and charge
AAAEEAEEA

SEQ ID NO: 386 - protein, 9 aa, Artificial Sequence
  synthetic exemplary 9-mer peptide from matrix library varying in hydrophobicity and charge
AAARRARRA

SEQ ID NO: 387 - protein, 9 aa, Artificial Sequence
  synthetic exemplary 9-mer peptide from matrix library varying in hydrophobicity and charge
WWEEWEEWW

SEQ ID NO: 388 - protein, 9 aa, Artificial Sequence
  synthetic exemplary 9-mer peptide from matrix library varying in hydrophobicity and charge
WWRRWRRWW

SEQ ID NO: 389 - protein, 9 aa, Artificial Sequence
  synthetic exemplary 9-mer peptide from matrix library varying in hydrophobicity and charge
AAEEAEEAA

SEQ ID NO: 390 - protein, 9 aa, Artificial Sequence
  synthetic exemplary 9-mer peptide from matrix library varying in hydrophobicity and charge
AARRARRAA

SEQ ID NO: 391 - protein, 9 aa, Artificial Sequence
  synthetic exemplary 9-mer peptide from matrix library varying in hydrophobicity and charge
WEEWEEWWW

SEQ ID NO: 392 - protein, 9 aa, Artificial Sequence
  synthetic exemplary 9-mer peptide from matrix library varying in hydrophobicity and charge
WRRWRRWWW

SEQ ID NO: 393 - protein, 9 aa, Artificial Sequence
  synthetic exemplary 9-mer peptide from matrix library varying in hydrophobicity and charge
AEEAEEAAA

SEQ ID NO: 394 - protein, 9 aa, Artificial Sequence
  synthetic exemplary 9-mer peptide from matrix library varying in hydrophobicity and charge
ARRARRAAA

SEQ ID NO: 395 - protein, 9 aa, Artificial Sequence
  synthetic exemplary 9-mer peptide from matrix library varying in hydrophobicity and charge
WWWEEEWEW

SEQ ID NO: 396 - protein, 9 aa, Artificial Sequence
  synthetic exemplary 9-mer peptide from matrix library varying in hydrophobicity and charge
WWWRRRWRW

SEQ ID NO: 397 - protein, 9 aa, Artificial Sequence
  synthetic exemplary 9-mer peptide from matrix library varying in hydrophobicity and charge
AAAEEEAEA

SEQ ID NO: 398 - protein, 9 aa, Artificial Sequence
  synthetic exemplary 9-mer peptide from matrix library varying in hydrophobicity and charge
AAARRRARA

SEQ ID NO: 399 - protein, 9 aa, Artificial Sequence
  synthetic exemplary 9-mer peptide from matrix library varying in hydrophobicity and charge
WWEEEWEWW

SEQ ID NO: 400 - protein, 9 aa, Artificial Sequence
  synthetic exemplary 9-mer peptide from matrix library varying in hydrophobicity and charge
WWRRRWRWW

SEQ ID NO: 401 - protein, 9 aa, Artificial Sequence
  synthetic exemplary 9-mer peptide from matrix library varying in hydrophobicity and charge
AAEEEAEAA

SEQ ID NO: 402 - protein, 9 aa, Artificial Sequence
  synthetic exemplary 9-mer peptide from matrix library varying in hydrophobicity and charge
AARRRARAA

SEQ ID NO: 403 - protein, 9 aa, Artificial Sequence
  synthetic exemplary 9-mer peptide from matrix library varying in hydrophobicity and charge
WEEEWEWWW

SEQ ID NO: 404 - protein, 9 aa, Artificial Sequence
  synthetic exemplary 9-mer peptide from matrix library varying in hydrophobicity and charge
WRRRWRWWW

SEQ ID NO: 405 - protein, 9 aa, Artificial Sequence
  synthetic exemplary 9-mer peptide from matrix library varying in hydrophobicity and charge
AEEEAEAAA

SEQ ID NO: 406 - protein, 9 aa, Artificial Sequence
  synthetic exemplary 9-mer peptide from matrix library varying in hydrophobicity and charge
ARRRARAAA

SEQ ID NO: 407 - protein, 9 aa, Artificial Sequence
  synthetic exemplary 9-mer peptide from matrix library varying in hydrophobicity and charge
WEEEWWEWW

SEQ ID NO: 408 - protein, 9 aa, Artificial Sequence
  synthetic exemplary 9-mer peptide from matrix library varying in hydrophobicity and charge
WRRRWWRWW

SEQ ID NO: 409 - protein, 9 aa, Artificial Sequence
  synthetic exemplary 9-mer peptide from matrix library varying in hydrophobicity and charge
AEEEAAEAA

SEQ ID NO: 410 - protein, 9 aa, Artificial Sequence
  synthetic exemplary 9-mer peptide from matrix library varying in hydrophobicity and charge
ARRRAARAA

SEQ ID NO: 411 - protein, 9 aa, Artificial Sequence
  synthetic exemplary 9-mer peptide from matrix library varying in hydrophobicity and charge
WEEEWWWEW

SEQ ID NO: 412 - protein, 9 aa, Artificial Sequence
  synthetic exemplary 9-mer peptide from matrix library varying in hydrophobicity and charge
WRRRWWWRW

SEQ ID NO: 413 - protein, 9 aa, Artificial Sequence
  synthetic exemplary 9-mer peptide from matrix library varying in hydrophobicity and charge
AEEEAAAEA

SEQ ID NO: 414 - protein, 9 aa, Artificial Sequence
  synthetic exemplary 9-mer peptide from matrix library varying in hydrophobicity and charge
ARRRAAARA

SEQ ID NO: 415 - protein, 9 aa, Artificial Sequence
  synthetic exemplary 9-mer peptide from matrix library varying in hydrophobicity and charge
WEEWWEEWW

SEQ ID NO: 416 - protein, 9 aa, Artificial Sequence
  synthetic exemplary 9-mer peptide from matrix library varying in hydrophobicity and charge
WRRWWRRWW

SEQ ID NO: 417 - protein, 9 aa, Artificial Sequence
  synthetic exemplary 9-mer peptide from matrix library varying in hydrophobicity and charge
AEEAAEEAA

SEQ ID NO: 418 - protein, 9 aa, Artificial Sequence
  synthetic exemplary 9-mer peptide from matrix library varying in hydrophobicity and charge
ARRAARRAA

SEQ ID NO: 419 - protein, 9 aa, Artificial Sequence
  synthetic exemplary 9-mer peptide from matrix library varying in hydrophobicity and charge
WEEWWWEEW

SEQ ID NO: 420 - protein, 9 aa, Artificial Sequence
  synthetic exemplary 9-mer peptide from matrix library varying in hydrophobicity and charge
WRRWWWRRW

SEQ ID NO: 421 - protein, 9 aa, Artificial Sequence
  synthetic exemplary 9-mer peptide from matrix library varying in hydrophobicity and charge
AEEAAAEEA

SEQ ID NO: 422 - protein, 9 aa, Artificial Sequence
  synthetic exemplary 9-mer peptide from matrix library varying in hydrophobicity and charge
ARRAAARRA

SEQ ID NO: 423 - protein, 5 aa, Artificial Sequence
  synthetic peptide with summed hydrophobicity value of 5.05
VWYAA

SEQ ID NO: 424 - protein, 5 aa, Artificial Sequence
  synthetic peptide with summed hydrophobicity value of 4.04
MAMYA

SEQ ID NO: 425 - protein, 4 aa, Artificial Sequence
  synthetic peptide with formal charge of +3
KKSK

SEQ ID NO: 426 - protein, 4 aa, Artificial Sequence
  synthetic peptide with formal charge of +2
KSQR

SEQ ID NO: 427 - protein, 4 aa, Artificial Sequence
  synthetic peptide with formal charge of +1
RTSQ

SEQ ID NO: 428 - protein, 4 aa, Artificial Sequence
  synthetic peptide with formal charge of 0
NTSQ

SEQ ID NO: 429 - protein, 9 aa, Artificial Sequence
  synthetic peptide with tissue-specific binding to tooth enamel and enamel-proximal region of dentino-enamel junction
AMKDAMERM

SEQ ID NO: 430 - protein, 8 aa, Artificial Sequence
  synthetic DYKDDDDK epitope tag
DYKDDDDK

SEQ ID NO: 431 - protein, 4 aa, Artificial Sequence
  synthetic His-4 epitope tag
HHHH

SEQ ID NO: 432 - protein, 5 aa, Artificial Sequence
  synthetic His-5 epitope tag
HHHHH

SEQ ID NO: 433 - protein, 6 aa, Artificial Sequence
  synthetic His-6 epitope tag
HHHHHH

SEQ ID NO: 434 - protein, 5 aa, Artificial Sequence
  synthetic peptide with summed hydrophobicity value of 3.03
AFAAA